SEQUENCE LISTING (13 sequences)

SEQ ID NO: 1 - DNA, 6230 bp, Artificial Sequence
  Plasmid pCMV-31int
cattcgccattcaggctgcgcaactgttgggaagggcgatcggtgcgggcctcttcgctattacgccagccaatacgcaaaccgcctctccccgcgcgttggccgattcattaatgcaggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatctaaaatacacaaacaattagaatcactagctcctgtgtataatattttcataaatcatactcagtaagcaaaactctcaagcagcaagcatatgcagctagtttaacacattatacacttaaaaattttatatttaccttagagctttaaatctctgtaggtagtttgtccaattatgtcacaccacagaagtaaggttccttcacaaagatcccaagctagcttataatacgactcactatagggagagagctatgacgtcgcatgcacgcgtaagcttgggcccctcgagggatccgggtgtctcgctacgccgctacgtcttccgtgccgtcctgggcgtcgtcttcgtcgtcgtcggtcggcggcttcgcccacgtgatcgaagcgcgcttctcgatgggcgttccctgccccctgcccgtagtcgacttcgtgacaacgatcttgtctacgaagagcccgacgaacacgcgcttgtcgtctactgacgcgcgcccccaccacgacttagggccggtcgggtcagcgtcggcgtcttcggggaaccattggtcaaggggaagcttcggggcttcggcggcttcaagttcggcaagccgctcttccgccccttgctgccggagcgtcagcgctgcctgttgcttccggaagtgcttcctgccaacgggtccgtcgtacgcgcctgccgcgcggtcttcgtacagctcttcaagggcgttcagggcgtcggcgcgctccgcaacaaggttcgcccgttcgccgctcttctcaggcgcctcagtgagcttgccgaagcgtcgggcggcttcccacagaagcgccaacgtctcttcgtcgccttcggcgtgcctgatcttgttgaagatgcgttccgcaacgaacttgtcgagtgccgccatgctgacgttgcacgtgccttcgtgctgcccaggtgcggacgggtcgaccaccttccggcgacggcagcggtaagagtccttgatcgattcttccccgcgcttcgaagtcatgacggcgccacactcgcagtacagcttgtccatggcggacagaatggcttgcccccgggaaagccccttgccgcgccccctgccgtccaaccacgcctgaagctcataccactcagcgggctcgatgatcggtccgcaatcaagctcgaccggccggagcgtgatcgggtcgcgctgaatgcggtaaccctcaatcttcgtggtcggcgtgccgtccggcttcttcttgtagatcacctcagcggcgaagcccgcaatacgcgggtcccgaaggattcgcataacggttgccgggtcccaggcgcttgaagcggtcttcttcccaatcgtctcgccccgggtcggcacggcgtcagcgtccatgcgcttacaaagccccgtgatgctgcccgggtgaatggcggcttgactgcccggcttgaagggaaggtgtttgtgcgtcttgatctcacgccaccaccaccggattacgtcgggctcgaactcgaagggtccggtaaggggagtggtcgagtgcgcaagcttgttgatgacgacattgaccattcggccgttgcgcgtgatctccttcgtctccgaaacaagctcgaagccgtaaggcgccttcccgccgacgtacccgcccaattcgcgctgaaggttcttcgtgtcgagaatcttcgccgacttcagcgaagattctttgtgcgacgcgtcgagccgcataatcaggtgaatcaggtccatgacgtttccctgccggaagacgccttcctgagtggaaacaatcgtcacgcccagggcgagcaattccgagacaatcggaatcgcgtccatgaccttcaggcgcgagaagcgcgacacgtcatagacaatgatcatgttgagccgcccggcgcggcattcgttcaggatgcgttcgaactccgggcgctccgccgtcccgaacgccgacgtgcccggcgcttcgctgaaatgcccgacgaacctgaaccggcccccgtcgcgctcgacttcgcgctgaaggtcggccgccttgtcttcgttggcgctacgctgtgtcgctgggcttgctgcgctcgaattctcgcgctcgcgcgactgacggtcgtaagcacccgcgtacgtgtccaccccggtcacaaccccttgtgtcatgtcggcgaccctacgactagtgagctcgtcgacccgggaattccggaccggtacctgcaggcgtaccttctatagtgtcacctaaatagctttttgcaaaagcctaggctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgacctgcacgatgctgtttcctgtgtgaaattgttatccgctcacaattccacacattatacgagccggaagctataaagtgtaaagcctggggtgcctaatgagtgaaagggcctcgtatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaacgcgcgaattgcaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccataacttcgtatagcatacattatacgaagttatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgccaggggtgggcacacatatttgataccagcgatccctacacagcacataattcaatgcgacttccctctatcgcacatcttagacctttattctccctccagcacacatcgaagctgccgagcaagccgttctcaccagtccaagacctggcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 2 - DNA, 5982 bp, Artificial Sequence
  Plasmid pCMV-luc-attB
ctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacattt

SEQ ID NO: 3 - DNA, 5924 bp, Artificial Sequence
  Plasmid pCMV-luc-attP
ctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcgactagtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgactagtacaagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacattt

SEQ ID NO: 4 - DNA, 5101 bp, Artificial Sequence
  Plasmid pCMV-pur-attB
ctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgact

SEQ ID NO: 5 - DNA, 5043 bp, Artificial Sequence
  Plasmid pCMV-pur-attP
ctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcgactagtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgactagtacaagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgact

SEQ ID NO: 6 - DNA, 5041 bp, Artificial Sequence
  Plasmid pCMV-EGFP-attB
ctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctcgagcatgcat

SEQ ID NO: 7 - DNA, 18116 bp, Artificial Sequence
  Plasmid p12.0lys-LSPIPNMM-CMV-pur-attB
gggctgcaggaattcgattgccgccttctttgatattcactctgttgtatttcatctcttcttgccgatgaaaggatataacagtctgtataacagtctgtgaggaaatacttggtatttcttctgatcagtgtttttataagtaatgttgaatattggataaggctgtgtgtcctttgtcttgggagacaaagcccacagcaggtggtggttggggtggtggcagctcagtgacaggagaggtttttttgcctgtttttttttttttttttttttttaagtaaggtgttcttttttcttagtaaattttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacacgagataccttattaagtttaggccagcttgatgctttattttttccctttgaagtagtgagcgttctctggtttttttcctttgaaactggtgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttcctttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttggctgctgccttcccgagatccaggacactaaactgcttctgcactgaggtataaatcgcttcagatcccagggaagtgcagatccacgtgcatattcttaaagaagaatgaatactttctaaaatattttggcataggaagcaagctgcatggatttgtttgggacttaaattattttggtaacggagtgcataggttttaaacacagttgcagcatgctaacgagtcacagcgtttatgcagaagtgatgcctggatgcctgttgcagctgtttacggcactgccttgcagtgagcattgcagataggggtggggtgctttgtgtcgtgttcccacacgctgccacacagccacctcccggaacacatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaaagcttctttggaaatacactgacttgattgaagtctcttgaagatagtaaacagtacttacctttgatcccaatgaaatcgagcatttcagttgtaaaagaattccgcctattcataccatgtaatgtaattttacacccccagtgctgacactttggaatatattcaagtaatagactttggcctcaccctcttgtgtactgtattttgtaatagaaaatattttaaactgtgcatatgattattacattatgaaagagacattctgctgatcttcaaatgtaagaaaatgaggagtgcgtgtgcttttataaatacaagtgattgcaaattagtgcaggtgtccttaaaaaaaaaaaaaaaaagtaatataaaaaggaccaggtgttttacaagtgaaatacattcctatttggtaaacagttacatttttatgaagattaccagcgctgctgactttctaaacataaggctgtattgtcttcctgtaccattgcatttcctcattcccaatttgcacaaggatgtctgggtaaactattcaagaaatggctttgaaatacagcatgggagcttgtctgagttggaatgcagagttgcactgcaaaatgtcaggaaatggatgtctctcagaatgcccaactccaaaggattttatatgtgtatatagtaagcagtttcctgattccagcaggccaaagagtctgctgaatgttgtgttgccggagacctgtatttctcaacaaggtaagatggtatcctagcaactgcggattttaatacattttcagcagaagtacttagttaatctctacctttagggatcgtttcatcatttttagatgttatacttgaaatactgcataacttttagctttcatgggttcctttttttcagcctttaggagactgttaagcaatttgctgtccaacttttgtgttggtcttaaactgcaatagtagtttaccttgtattgaagaaataaagaccatttttatattaaaaaatacttttgtctgtcttcattttgacttgtctgatatccttgcagtgcccattatgtcagttctgtcagatattcagacatcaaaacttaacgtgagctcagtggagttacagctgcggttttgatgctgttattatttctgaaactagaaatgatgttgtcttcatctgctcatcaaacacttcatgcagagtgtaaggctagtgagaaatgcatacatttattgatacttttttaaagtcaactttttatcagatttttttttcatttggaaatatattgttttctagactgcatagcttctgaatctgaaatgcagtctgattggcatgaagaagcacagcactcttcatcttacttaaacttcattttggaatgaaggaagttaagcaagggcacaggtccatgaaatagagacagtgcgctcaggagaaagtgaacctggatttctttggctagtgttctaaatctgtagtgaggaaagtaacacccgattccttgaaagggctccagctttaatgcttccaaattgaaggtggcaggcaacttggccactggttatttactgcattatgtctcagtttcgcagctaacctggcttctccactattgagcatggactatagcctggcttcagaggccaggtgaaggttgggatgggtggaaggagtgctgggctgtggctggggggactgtggggactccaagctgagcttggggtgggcagcacagggaaaagtgtgggtaactatttttaagtactgtgttgcaaacgtctcatctgcaaatacgtagggtgtgtactctcgaagattaacagtgtgggttcagtaatatatggatgaattcacagtggaagcattcaagggtagatcatctaacgacaccagatcatcaagctatgattggaagcggtatcagaagagcgaggaaggtaagcagtcttcatatgttttccctccacgtaaagcagtctgggaaagtagcaccccttgagcagagacaaggaaataattcaggagcatgtgctaggagaactttcttgctgaattctacttgcaagagctttgatgcctggcttctggtgccttctgcagcacctgcaaggcccagagcctgtggtgagctggagggaaagattctgctcaagtccaagcttcagcaggtcattgtctttgcttcttcccccagcactgtgcagcagagtggaactgatgtcgaagcctcctgtccactacctgttgctgcaggcagactgctctcagaaaaagagagctaactctatgccatagtctgaaggtaaaatgggttttaaaaaagaaaacacaaaggcaaaaccggctgccccatgagaagaaagcagtggtaaacatggtagaaaaggtgcagaagcccccaggcagtgtgacaggcccctcctgccacctagaggcgggaacaagcttccctgcctagggctctgcccgcgaagtgcgtgtttctttggtgggttttgtttggcgtttggttttgagatttagacacaagggaagcctgaaaggaggtgttgggcactattttggtttgtaaagcctgtacttcaaatatatattttgtgagggagtgtagcgaattggccaatttaaaataaagttgcaagagattgaaggctgagtagttgagagggtaacacgtttaatgagatcttctgaaactactgcttctaaacacttgtttgagtggtgagaccttggataggtgagtgctcttgttacatgtctgatgcacttgcttgtccttttccatccacatccatgcattccacatccacgcatttgtcacttatcccatatctgtcatatctgacatacctgtctcttcgtcacttggtcagaagaaacagatgtgataatccccagccgccccaagtttgagaagatggcagttgcttctttccctttttcctgctaagtaaggattttctcctggctttgacacctcacgaaatagtcttcctgccttacattctgggcattatttcaaatatctttggagtgcgctgctctcaagtttgtgtcttcctactcttagagtgaatgctcttagagtgaaagagaaggaagagaagatgttggccgcagttctctgatgaacacacctctgaataatggccaaaggtgggtgggtttctctgaggaacgggcagcgtttgcctctgaaagcaaggagctctgcggagttgcagttattttgcaactgatggtggaactggtgcttaaagcagattccctaggttccctgctacttcttttccttcttggcagtcagtttatttctgacagacaaacagccacccccactgcaggcttagaaagtatgtggctctgcctgggtgtgttacagctctgccctggtgaaaggggattaaaacgggcaccattcatcccaaacaggatcctcattcatggatcaagctgtaaggaacttgggctccaacctcaaaacattaattggagtacgaatgtaattaaaactgcattctcgcattcctaagtcatttagtctggactctgcagcatgtaggtcggcagctcccactttctcaaagaccactgatggaggagtagtaaaaatggagaccgattcagaacaaccaacggagtgttgccgaagaaactgatggaaataatgcatgaattgtgtggtggacattttttttaaatacataaactacttcaaatgaggtcggagaaggtcagtgttttattagcagccataaaaccaggtgagcgagtaccatttttctctacaagaaaaacgattctgagctctgcgtaagtataagttctccatagcggctgaagctcccccctggctgcctgccatctcagctggagtgcagtgccatttccttggggtttctctcacagcagtaatgggacaatacttcacaaaaattctttcttttcctgtcatgtgggatccctactgtgccctcctggttttacgttaccccctgactgttccattcagcggtttggaaagagaaaaagaatttggaaataaaacatgtctacgttatcacctcctccagcattttggtttttaattatgtcaataactggcttagatttggaaatgagagggggttgggtgtattaccgaggaacaaaggaaggcttatataaactcaagtcttttatttagagaactggcaagctgtcaaaaacaaaaaggccttaccaccaaattaagtgaatagccgctatagccagcagggccagcacgagggatggtgcactgctggcactatgccacggcctgcttgtgactctgagagcaactgctttggaaatgacagcacttggtgcaatttcctttgtttcagaatgcgtagagcgtgtgcttggcgacagtttttctagttaggccacttcttttttccttctctcctcattctcctaagcatgtctccatgctggtaatcccagtcaagtgaacgttcaaacaatgaatccatcactgtaggattctcgtggtgatcaaatctttgtgtgaggtctataaaatatggaagcttatttatttttcgttcttccatatcagtcttctctatgacaattcacatccaccacagcaaattaaaggtgaaggaggctggtgggatgaagagggtcttctagctttacgttcttccttgcaaggccacaggaaaatgctgagagctgtagaatacagcctggggtaagaagttcagtctcctgctgggacagctaaccgcatcttataaccccttctgagactcatcttaggaccaaatagggtctatctggggtttttgttcctgctgttcctcctggaaggctatctcactatttcactgctcccacggttacaaaccaaagatacagcctgaattttttctaggccacattacataaatttgacctggtaccaatattgttctctatatagttatttccttccccactgtgtttaaccccttaaggcattcagaacaactagaatcatagaatggtttggattggaaggggccttaaacatcatccatttccaaccctctgccatgggctgcttgccacccactggctcaggctgcccagggccccatccagcctggccttgagcacctccagggatggggcacccacagcttctctgggcagcctgtgccaacacctcaccactctctgggtaaagaattctcttttaacatctaatctaaatctcttctcttttagtttaaagccattcctctttttcccgttgctatctgtccaagaaatgtgtattggtctccctcctgcttataagcaggaagtactggaaggctgcagtgaggtctccccacagccttctcttctccaggctgaacaagcccagctccttcagcctgtcttcgtaggagatcatcttagtggccctcctctggacccattccaacagttccacggctttcttgtggagccccaggtctggatgcagtacttcagatggggccttacaaaggcagagcagatggggacaatcgcttacccctccctgctggctgcccctgttttgatgcagcccagggtactgttggcctttcaggctcccagaccccttgctgatttgtgtcaagcttttcatccaccagaacccacgcttcctggttaatacttctgccctcacttctgtaagcttgtttcaggagacttccattctttaggacagactgtgttacacctacctgccctattcttgcatatatacatttcagttcatgtttcctgtaacaggacagaatatgtattcctctaacaaaaatacatgcagaattcctagtgccatctcagtagggttttcatggcagtattagcacatagtcaatttgctgcaagtaccttccaagctgcggcctcccataaatcctgtatttgggatcagttaccttttggggtaagcttttgtatctgcagagaccctgggggttctgatgtgcttcagctctgctctgttctgactgcaccattttctagatcacccagttgttcctgtacaacttccttgtcctccatcctttcccagcttgtatctttgacaaatacaggcctatttttgtgtttgcttcagcagccatttaattcttcagtgtcatcttgttctgttgatgccactggaacaggattttcagcagtcttgcaaagaacatctagctgaaaactttctgccattcaatattcttaccagttcttcttgtttgaggtgagccataaattactagaacttcgtcactgacaagtttatgcattttattacttctattatgtacttactttgacataacacagacacgcacatattttgctgggatttccacagtgtctctgtgtccttcacatggttttactgtcatacttccgttataaccttggcaatctgcccagctgcccatcacaagaaaagagattccttttttattacttctcttcagccaataaacaaaatgtgagaagcccaaacaagaacttgtggggcaggctgccatcaagggagagacagctgaagggttgtgtagctcaatagaattaagaaataataaagctgtgtcagacagttttgcctgatttatacaggcacgccccaagccagagaggctgtctgccaaggccaccttgcagtccttggtttgtaagataagtcataggtaacttttctggtgaattgcgtggagaatcatgatggcagttcttgctgtttactatggtaagatgctaaaataggagacagcaaagtaacacttgctgctgtaggtgctctgctatccagacagcgatggcactcgcacaccaagatgagggatgctcccagctgacggatgctggggcagtaacagtgggtcccatgctgcctgctcattagcatcacctcagccctcaccagcccatcagaaggatcatcccaagctgaggaaagttgctcatcttcttcacatcatcaaacctttggcctgactgatgcctcccggatgcttaaatgtggtcactgacatctttatttttctatgatttcaagtcagaacctccggatcaggagggaacacatagtgggaatgtaccctcagctccaaggccagatcttccttcaatgatcatgcatgctacttaggaaggtgtgtgtgtgtgaatgtagaattgcctttgttattttttcttcctgctgtcaggaacattttgaataccagagaaaaagaaaagtgctcttcttggcatgggaggagttgtcacacttgcaaaataaaggatgcagtcccaaatgttcataatctcagggtctgaaggaggatcagaaactgtgtatacaatttcaggcttctctgaatgcagcttttgaaagctgttcctggccgaggcagtactagtcagaaccctcggaaacaggaacaaatgtcttcaaggtgcagcaggaggaaacaccttgcccatcatgaaagtgaataaccactgccgctgaaggaatccagctcctgtttgagcaggtgctgcacactcccacactgaaacaacagttcatttttataggacttccaggaaggatcttcttcttaagcttcttaattatggtacatctccagttggcagatgactatgactactgacaggagaatgaggaactagctgggaatatttctgtttgaccaccatggagtcacccatttctttactggtatttggaaataataattctgaattgcaaagcaggagttagcgaagatcttcatttcttccatgttggtgacagcacagttctggctatgaaagtctgcttacaaggaagaggataaaaatcatagggataataaatctaagtttgaagacaatgaggttttagctgcatttgacatgaagaaattgagacctctactggatagctatggtatttacgtgtctttttgcttagttacttattgaccccagctgaggtcaagtatgaactcaggtctctcgggctactggcatggattgattacatacaactgtaattttagcagtgatttagggtttatgagtacttttgcagtaaatcatagggttagtaatgttaatctcagggaaaaaaaaaaaaagccaaccctgacagacatcccagctcaggtggaaatcaaggatcacagctcagtgcggtcccagagaacacagggactcttctcttaggacctttatgtacagggcctcaagataactgatgttagtcagaagactttccattctggccacagttcagctgaggcaatcctggaattttctctccgctgcacagttccagtcatcccagtttgtacagttctggcactttttgggtcaggccgtgatccaaggagcagaagttccagctatggtcagggagtgcctgaccgtcccaactcactgcactcaaacaaaggcgaaaccacaagagtggcttttgttgaaattgcagtgtggcccagaggggctgcaccagtactggattgaccacgaggcaacattaatcctcagcaagtgcaatttgcagccattaaattgaactaactgatactacaatgcaatcagtatcaacaagtggtttggcttggaagatggagtctaggggctctacaggagtagctactctctaatggagttgcattttgaagcaggacactgtgaaaagctggcctcctaaagaggctgctaaacattagggtcaattttccagtgcactttctgaagtgtctgcagttccccatgcaaagctgcccaaacatagcacttccaattgaatacaattatatgcaggcgtactgcttcttgccagcactgtccttctcaaatgaactcaacaaacaatttcaaagtctagtagaaagtaacaagctttgaatgtcattaaaaagtatatctgctttcagtagttcagcttatttatgcccactagaaacatcttgtacaagctgaacactggggctccagattagtggtaaaacctactttatacaatcatagaatcatagaatggcctgggttggaagggaccccaaggatcatgaagatccaacacccccgccacaggcagggccaccaacctccagatctggtactagaccaggcagcccagggctccatccaacctggccatgaacacctccagggatggagcatccacaacctctctgggcagcctgtgccagcacctcaccaccctctctgtgaagaacttttccctgacatccaatctaagccttccctccttgaggttagatccactcccccttgtgctatcactgtctactcttgtaaaaagttgattctcctcctttttggaaggttgcaatgaggtctccttgcagccttcttctcttctgcaggatgaacaagcccagctccctcagcctgtctttataggagaggtgctccagccctctgatcatctttgtggccctcctctggacccgctccaagagctccacatctttcctgtactgggggccccaggcctgaatgcagtactccagatggggcctcaaaagagcagagtaaagagggacaatcaccttcctcaccctgctggccagccctcttctgatggagccctggatacaactggctttctgagctgcaacttctccttatcagttccactattaaaacaggaacaatacaacaggtgctgatggccagtgcagagtttttcacacttcttcatttcggtagatcttagatgaggaacgttgaagttgtgcttctgcgtgtgcttcttcctcctcaaatactcctgcctgatacctcaccccacctgccactgaatggctccatggccccctgcagccagggccctgatgaacccggcactgcttcagatgctgtttaatagcacagtatgaccaagttgcacctatgaatacacaaacaatgtgttgcatccttcagcacttgagaagaagagccaaatttgcattgtcaggaaatggtttagtaattctgccaattaaaacttgtttatctaccatggctgtttttatggctgttagtagtggtacactgatgatgaacaatggctatgcagtaaaatcaagactgtagatattgcaacagactataaaattcctctgtggcttagccaatgtggtacttcccacattgtataagaaatttggcaagtttagagcaatgtttgaagtgttgggaaatttctgtatactcaagagggcgtttttgacaactgtagaacagaggaatcaaaagggggtgggaggaagttaaaagaagaggcaggtgcaagagagcttgcagtcccgctgtgtgtacgacactggcaacatgaggtctttgctaatcttggtgctttgcttcctgcccctggctgccttagggtgcgatctgcctcagacccacagcctgggcagcaggaggaccctgatgctgctggctcagatgaggagaatcagcctgtttagctgcctgaaggataggcacgattttggctttcctcaagaggagtttggcaaccagtttcagaaggctgagaccatccctgtgctgcacgagatgatccagcagatctttaacctgtttagcaccaaggatagcagcgctgcttgggatgagaccctgctggataagttttacaccgagctgtaccagcagctgaacgatctggaggcttgcgtgatccagggcgtgggcgtgaccgagacccctctgatgaaggaggatagcatcctggctgtgaggaagtactttcagaggatcaccctgtacctgaaggagaagaagtacagcccctgcgcttgggaagtcgtgagggctgagatcatgaggagctttagcctgagcaccaacctgcaagagagcttgaggtctaaggagtaaaaagtctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgctctagaactagtggatcccccggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatg

SEQ ID NO: 8 - DNA, 17402 bp, Artificial Sequence
  Plasmid pOMIFN-Ins-CMV-pur-attB
ggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctgggtaccgggccccccctcgactagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggctctagtcgaggtcgacggtatcgataagcttgattaggcagagcaataggactctcaacctcgtgagtatggcagcatgttaactctgcactggagtccagcgtgggaaacaatctgccttgcacatgagtcttcgtgggccaatattccccaacggttttccttcagcttgtcttgtctcctaagctctcaaaacacctttttggtgaataaactcacttggcaacgtttatctgtcttaccttagtgtcacgtttcatccctattcccctttctcctcctccgtgtggtacacagtggtgcacactggttcttctgttgatgttctgctctgacagccaatgtgggtaaagttcttcctgccacgtgtctgtgttgttttcacttcaaaaagggccctgggctccccttggagctctcaggcatttccttaatcatcacagtcacgctggcaggattagtccctcctaaaccttagaatgacctgaacgtgtgctccctctttgtagtcagtgcagggagacgtttgcctcaagatcagggtccatctcacccacagggccattcccaagatgaggtggatggtttactctcacaaaaagttttcttatgtttggctagaaaggagaactcactgcctacctgtgaattcccctagtcctggttctgctgccactgctgcctgtgcagcctgtcccatggagggggcagcaactgctgtcacaaaggtgatcccaccctgtctccactgaaatgacctcagtgccacgtgttgtatagggtataaagtacgggagggggatgcccggctcccttcagggttgcagagcagaagtgtctgtgtatagagtgtgtcttaatctattaatgtaacagaacaacttcagtcctagtgttttgtgggctggaattgcccatgtggtagggacaggcctgctaaatcactgcaatcgcctatgttctgaaggtatttgggaaagaaagggatttgggggattgcctgtgattggctttaattgaatggcaaatcacaggaaagcagttctgctcaacagttggttgtttcagccaattcttgcagccaaagagccgggtgcccagcgatataatagttgtcacttgtgtctgtatggatgacagggaggtagggtgacctgaggaccaccctccagcttctgctagcgtaggtacagtcaccacctccagctccacacgagtcccatcgtggtttaccaaagaaacacaattatttggaccagtttggaaagtcacccgctgaattgtgaggctagattaatagagctgaagagcaaatgttcccaacttggagatactagttggtattagtatcagaggaacagggccatagcacctccatgctattagattccggctggcatgtacttttcaagatgatttgtaactaacaatggcttattgtgcttgtcttaagtctgtgtcctaatgtaaatgttcctttggtttatataaccttcttgccatttgctcttcaggtgttcttgcagaacactggctgctttaatctagtttaactgttgcttgattattcttagggataagatctgaataaactttttgtggctttggcagactttagcttgggcttagctcccacattagcttttgctgccttttctgtgaagctatcaagatcctactcaatgacattagctgggtgcaggtgtaccaaatcctgctctgtggaacacattgtctgatgataccgaaggcaaacgtgaactcaaagaggcacagagttaagaagaagtctgtgcaattcagaggaaaagccaaagtggccattagacacactttccatgcagcatttgccagtaggtttcatataaaactacaaaatggaataaaccactacaaatgggaaaagcctgatactagaatttaaatattcacccaggctcaaggggtgtttcatggagtaatatcactctataaaagtagggcagccaattattcacagacaaagcttttttttttctgtgctgcagtgctgtttttcggctgatccagggttacttattgtgggtctgagagctgaatgatttctccttgtgtcatgttggtgaaggagatatggccagggggagatgagcatgttcaagaggaaacgttgcattttggtggcttgggagaaaggtagaacgatatcaggtccatagtgtcactaagagatctgaaggatggttttacagaacagttgacttggctgggtgcaggcttggctgtaaatggatggaaggatggacagatgggtggacagagatttctgtgcaggagatcatctcctgagctcggtgcttgacagactgcagatccatcccataaccttctccagcatgagagcgcggggagctttggtactgttcagtctgctgcttgttgcttcctgggtgcacagtggtgattttcttactcacacagggcaaaaacctgagcagcttcaaagtgaacaggttgctctcataggccattcagttgtcaagatgaggtttttggtttcttgttttgtaaggtgggaagaagcactgaaggatcagttgcgagggcaggggtttagcactgttcagagaagtcttattttaactcctctcatgaacaaaaagagatgcaggtgcagattctggcaagcatgcagtgaaggagaaagccctgaatttctgatatatgtgcaatgttgggcacctaacattccccgctgaagcacagcagctccagctccatgcagtactcacagctggtgcagccctcggctccagggtctgagcagtgctgggactcacgaggttccatgtctttcacactgataatggtccaatttctggaatgggtgcccatccttggaggtccccaaggccaggctggctgcgtctccgagcagcccgatctggtggtgagtagccagcccatggcaggagttagagcctgatggtctttaaggtcccttccaacctaagccatcctacgattctaggaatcatgacttgtgagtgtgtattgcagaggcaatattttaaagttataaatgttttctccccttccttgtttgtcaaagttatcttgatcgccttatcaatgcttttggagtctccagtcatttttcttacamcaaaaagaggaggaagaatgaagagaatcatttaatttcttgattgaatagtaggattcagaaagctgtacgtaatgccgtctctttgtatcgagctgtaaggtttctcatcatttatcagcgtggtacatatcagcacttttccatctgatgtggaaaaaaaaatccttatcatctacagtctctgtacctaaacatcgctcagactctttaccaaaaaagctataggttttaaaactacatctgctgataatttgccttgttttagctcttcttccatatgctgcgtttgtgagaggtgcgtggatgggcctaaactctcagctgctgagcttgatgggtgcttaagaatgaagcactcactgctgaaactgttttcatttcacaggaatgttttagtggcattgtttttataactacatattcctcagataaatgaaatccagaaataattatgcaaactcactgcatccgttgcacaggtctttatctgctagcaaaggaaataatttggggatggcaaaaacattccttcagacatctatatttaaaggaatataatcctggtacccacccacttcatccctcattatgttcacactcagagatactcattctcttgttgttatcatttgatagcgttttctttggttctttgccacgctctgggctatggctgcacgctctgcactgatcagcaagtagatgcgagggaagcagcagtgagaggggctgccctcagctggcacccagccgctcagcctaggaggggaccttgcctttccaccagctgaggtgcagccctacaagcttacacgtgctgcgagcaggtgagcaaagggagtcttcatggtgtgtttcttgctgcccggaagcaaaactttactttcattcattccccttgaagaatgaggaatgtttggaaacggactgctttacgttcaatttctctcttccctttaaggctcagccaggggccattgctgaggacggcatcggggccccctggaccaaatctgtggcacagatggtttcacttacatcagtggatgtgggatctgcgcctgtaatgtgtccttctgaaggaaggaacgtgccttccaagtgccagccccacagcccccagcccctccctgtgctgctccaattcatctcctcttcctccttctccctttgctgtttgtgctcgggtagaaatcatgaagatttagaagagaaaacaaaataactggagtggaaacccaggtgatgcagttcattcagctgtcataggtttgtcgttgctataggtctgtatcagagatgctarcaccactttgctgtcggtgcttaactcgggtgaactctccttcactcgcatcatttgcgggccttatttacatccccagcatccatcaccctctgggaaaatgggcgcactggatctctaatggaagactttccctctttcagagcctgtgggatgtgcagtgacaagaaacgtggaggggctgagcagcagcactgcccccagggagcaggagcggatgccatcggtggcagcatcccaaatgatgtcagcggatgctgagcaggcagcggacgaacggacagaagcgatgcgtacaccttctgttgacatggtatttggcagcgatttaacactcgcttcctagtcctgctattctccacaggctgcattcaaatgaacgaagggaagggaggcaaaaagatgcaaaatccgagacaagcagcagaaatatttcttcgctacggaagcgtgcgcaaacaaccttctccaacagcaccagaagagcacagcgtaacctttttcaagaccagaaaaggaaattcacaaagcctctgtggataccagcgcgttcagctctcctgatagcagatttcttgtcaggttgcgaatggggtatggtgccaggaggtgcagggaccatatgatcatatacagcacagcagtcattgtgcatgtattaatatatattgagtagcagtgttactttgccaaagcaatagttcagagatgagtcctgctgcatacctctatcttaaaactaacttataaatagtaaaaccttctcagttcagccacgtgctcctctctgtcagcaccaatggtgcttcgcctgcacccagctgcaaggaatcagcccgtgatctcattaacactcagctctgcaggataaattagattgttccactctcttttgttgttaattacgacggaacaattgttcagtgctgatggtcctaattgtcagctacagaaaacgtctccatgcagttccttctgcgccagcaaactgtccaggctatagcaccgtgatgcatgctacctctcactccatccttcttctctttcccaccagggagagctgtgtgttttcactctcagccactctgaacaataccaaactgctacgcactgcctccctcggaaagagaatccccttgttgcttttttatttacaggatccttcttaaaaagcagaccatcattcactgcaaacccagagcttcatgcctctccttccacaaccgaaaacagccggcttcatttgtcttttttaaatgctgttttccaggtgaattttggccagcgtgttggctgagatccaggagcacgtgtcagctttctgctctcattgctcctgttctgcattgcctctttctggggtttccaagagggggggagactttgcgcggggatgagataatgccccttttcttagggtggctgctgggcagcagagtggctctgggtcactgtggcaccaatgggaggcaccagtgggggtgtgttttgtgcaggggggaagcattcacagaatggggctgatcctgaagcttgcagtccaaggctttgtctgtgtacccagtgaaatccttcctctgttacataaagcccagataggactcagaaatgtagtcattccagcccccctcttcctcagatctggagcagcacttgtttgcagccagtcctccccaaaatgcacagacctcgccgagtggagggagatgtaaacagcgaaggttaattacctccttgtcaaaaacactttgtggtccatagatgtttctgtcaatcttacaaaacagaaccgagaggcagcgagcactgaagagcgtgttcccatgctgagttaatgagacttggcagctcgctgtgcagagatgatccctgtgcttcatgggaggctgtaacctgtctccccatcgccttcacaccgcagtgctgtcctggacacctcaccctccataagctgtaggatgcagctgcccagggatcaagagacttttcctaaggctcttaggactcatctttgccgctcagtagcgtgcagcaattactcatcccaactatactgaatgggtttctgccagctctgcttgtttgtcaataagcatttcttcattttgcctctaagtttctctcagcagcaccgctctgggtgacctgagtggccacctggaacccgaggggcacagccaccacctccctgttgctgctgctccagggactcatgtgctgctggatggggggaagcatgaagttcctcacccagacacctgggttgcaatggctgcagcgtgctcttcttggtatgcagattgtttccagccattacttgtagaaatgtgctgtggaagccctttgtatctctttctgtggcccttcagcaaaagctgtgggaaagctctgaggctgctttcttgggtcgtggaggaattgtatgttccttctttaacaaaaattatccttaggagagagcactgtgcaagcattgtgcacataaaacaattcaggttgaaagggctctctggaggtttccagcctgactactgctcgaagcaaggccaggttcaaagatggctcaggatgctgtgtgccttcctgattatctgtgccaccaatggaggagattcacagccactctgcttcccgtgccactcatggagaggaatattcccttatattcagatagaatgttatcctttagctcagccttccctataaccccatgagggagctgcagatccccatactctccccttctctggggtgaaggccgtgtcccccagccccccttcccaccctgtgccctaagcagcccgctggcctctgctggatgtgtgcctatatgtcaatgcctgtccttgcagtccagcctgggacatttaattcatcaccagggtaatgtggaactgtgtcatcttcccctgcagggtacaaagttctgcacggggtcctttcggttcaggaaaaccttcactggtgctacctgaatcaagctctatttaataagttcataagcacatggatgtgttttcctagagatacgttttaatggtatcagtgatttttatttgctttgttgcttacttcaaacagtgcctttgggcaggaggtgagggacgggtctgccgttggctctgcagtgatttctccaggcgtgtggctcaggtcagatagtggtcactctgtggccagaagaaggacaaagatggaaattgcagattgagtcacgttaagcaggcatcttggagtgatttgaggcagtttcatgaaagagctacgaccacttattgttgttttccccttttacaacagaagttttcatcaaaataacgtggcaaagcccaggaatgtttgggaaaagtgtagttaaatgttttgtaattcatttgtcggagtgctaccagctaagaaaaaagtcctacctttggtatggtagtcctgcagagaatacaacatcaatattagtttggaaaaaaacaccaccaccaccagaaactgtaatggaaaatgtaaaccaagaaattccttgggtaagagagaaaggatgtcgtatactggccaagtcctgcccagctgtcagcctgctgaccctctgcagttcaggaccatgaaacgtggcactgtaagacgtgtcccctgcctttgcttgcccacagatctctgcccttgtgctgactcctgcacacaagagcatttccctgtagccaaacagcgattagccataagctgcacctgactttgaggattaagagtttgcaattaagtggattgcagcaggagatcagtggcagggttgcagatgaaatccttttctaggggtagctaagggctgagcaacctgtcctacagcacaagccaaaccagccaagggttttcctgtgctgttcacagaggcagggccagctggagctggaggaggttgtgctgggacccttctccctgtgctgagaatggagtgatttctgggtgctgttcctgtggcttgcactgagcagctcaagggagatcggtgctcctcatgcagtgccaaaactcgtgtttgatgcagaaagatggatgtgcacctccctcctgctaatgcagccgtgagcttatgaaggcaatgagccctcagtgcagcaggagctgtagtgcactcctgtaggtgctagggaaaatctctggttcccagggatgcattcataagggcaatatatcttgaggctgcgccaaatctttctgaaatattcatgcgtgttcccttaatttatagaaacaaacacagcagaataattattccaatgcctcccctcgaaggaaacccatatttccatgtagaaatgtaacctatatacacacagccatgctgcatccttcagaacgtgccagtgctcatctcccatggcaaaatactacaggtattctcactatgttggacctgtgaaaggaaccatggtaagaaacttcggttaaaggtatggctgcaaaactactcataccaaaacagcagagctccagacctcctcttaggaaagagccacttggagagggatggtgtgaaggctggaggtgagagacagagcctgtcccagttttcctgtctctattttctgaaacgtttgcaggaggaaaggacaactgtactttcaggcatagctggtgccctcacgtaaataagttccccgaacttctgtgtcatttgttcttaagatgctttggcagaacactttgagtcaattcgcttaactgtgactaggtctgtaaataagtgctccctgctgataaggttcaagtgacatttttagtggtatttgacagcatttaccttgctttcaagtcttctaccaagctcttctatacttaagcagtgaaaccgccaagaaacccttccttttatcaagctagtgctaaataccattaacttcataggttagatacggtgctgccagcttcacctggcagtggttggtcagttctgctggtgacaaagcctccctggcctgtgcttttacctagaggtgaatatccaagaatgcagaactgcatggaaagcagagctgcaggcacgatggtgctgagccttagctgcttcctgctgggagatgtggatgcagagacgaatgaaggacctgtcccttactcccctcagcattctgtgctatttagggttctaccagagtccttaagaggttttttttttttttggtccaaaagtctgtttgtttggttttgaccactgagagcatgtgacacttgtctcaagctattaaccaagtgtccagccaaaatcaattgcctgggagacgcagaccattacctggaggtcaggacctcaataaatattaccagcctcattgtgccgctgacagattcagctggctgctccgtgttccagtccaacagttcggacgccacgtttgtatatatttgcaggcagcctcggggggaccatctcaggagcagagcaccggcagccgcctgcagagccgggcagtacctcaccatggctttgacctttgccttactggtggctctcctggtgctgagctgcaagagcagctgctctgtgggctgcgatctgcctcagacccacagcctgggcagcaggaggaccctgatgctgctggctcagatgaggagaatcagcctgtttagctgcctgaaggataggcacgattttggctttcctcaagaggagtttggcaaccagtttcagaaggctgagaccatccctgtgctgcacgagatgatccagcagatctttaacctgtttagcaccaaggatagcagcgctgcttgggatgagaccctgctggataagttttacaccgagctgtaccagcagctgaacgatctggaggcttgcgtgatccagggcgtgggcgtgaccgagacccctctgatgaaggaggatagcatcctggctgtgaggaagtactttcagaggatcaccctgtacctgaaggagaagaagtacagcccctgcgcttgggaagtcgtgagggctgagatcatgaggagctttagcctgagcaccaacctgcaagagagcttgaggtctaaggagtaaaaagtctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataccgtcgacctcgactagagcggccactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagc

SEQ ID NO: 9 - DNA, 5172 bp, Artificial Sequence
  Plasmid pRSV-Int
ctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgctaggggtctaggatcgattctaggaattctctagccgcggtctagggatcccggcgcgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagcttgcatgcctgcaggtaccggtccggaattcccgggtcgacgagctcactagtcgtagggtcgccgacatgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtagcgagacacccggatccctcgaggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggtgcccagtcatagccgaatagcctctccacccaagcggccggagaacctgcgtgcaatccactgggggcgcg

SEQ ID NO: 10 - DNA, 6233 bp, Artificial Sequence
  Plasmid pCR-XL-TOPO-CMV-pur-attB
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacgcgttagaatactcaagctatgcatcaagcttggtaccgagctcggatccactagtaacggccgccagtgtgctggaattcgcccttggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 11 - DNA, 234 bp, Artificial Sequence
  attP containing polynucleotide
gactagtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgactagtaca

SEQ ID NO: 12 - DNA, 26 bp, Artificial Sequence
  Primer attB-for
taccgtcgacgatgtaggtcacggtc

SEQ ID NO: 13 - protein, 11 aa, SV40
CGGPKKKRKVG

(19) United States
(12) Patent Application Publication
(10) Pub. No.: US 2004/0255345 A1
(43) Pub. Date: Dec. 16, 2004

(54) Production of transgenic avians

(76) Inventors: Jeffrey C. Rapp, Athens, GA (US); Leandro Christmann, Watkinsville, GA (US); Alex J. Harvey, Athens, GA (US); Markley C. Leavitt, Watkinsville, GA (US)

Correspondence Address: AVIGENICS, INC., 111 RIVERBEND ROAD, ATHENS, GA, 30605, US

(21) Appl. No.: 10/811,136

(22) Filed: Mar. 26, 2004

Related U.S. Application Data
(63) Continuation-in-part of application No. 10/790,455, filed on Mar. 1, 2004.
(60) Provisional application No. 60/453,126, filed on Mar. 7, 2003.
(60) Provisional application No. 60/490,452, filed on Jul. 28, 2003.
(60) Provisional application No. 60/536,677, filed on Jan. 15, 2004.
(60) Provisional application No. 60/458,014, filed on Mar. 27, 2003.

(51) Int. Cl. A01K 67/027

(52) U.S. Cl. 800/19; 119/300

(57) ABSTRACT

The invention provides methods for integrating a heterologous polynucleotide into the genome of an avian cell. The methods deliver to an avian cell a polynucleotide and a source of integrase activity that mediates recombination between the polynucleotide and the genomic DNA of the avian cell. The invention provides modified avian or artificial chromosomes as vectors to shuttle transgenes or gene clusters into an avian genome. Another aspect of the invention are avian cells genetically modified with a transgene vector. One cell line for the delivery and integration of a transgene comprises a heterologous attP site and, optionally, a region for expressing the integrase. Methods are also included for the production of a heterologous polypeptide by transgenic avian tissue involve integrating a heterologous polynucleotide into the avian genome. The present invention also relates to methods of producing transgenic chickens which include introducing into an avian cell a nucleic acid comprising a transgene and an integrase activity in addition to a cationic polymer and/or a nuclear localization signal and introducing the avian cell into a recipient avian wherein the recipient avian produces an offspring which includes the transgene. Also included are methods of dispersing a nucleic acid in a cell.

The present application is a continuation-in-part of U.S. patent application Ser. No. (to be assigned), filed Mar. 7, 2004, which is hereby incorporated by reference in its entirety and claims priority from U.S. provisional patent application Ser. No. 60/458,014, filed Mar. 27, 2003, which is hereby incorporated by reference in its entirety.

### FIELD OF THE INVENTION

The present invention relates to the field of biotechnology, and more specifically to the field of avian genome modification. Disclosed herein are compositions, vectors, and methods of use thereof, for the generation of genetically transformed avian cells and transgenic birds.

### BACKGROUND

Transgenic technology to convert animals into “bioreactors” for the production of specific proteins or other substances of pharmaceutical interest (Gordon et al., 1987, Biotechnology 5: 1183-1187; Wilmut et al., 1990, Theriogenology 33: 113-123) offers significant advantages over more conventional methods of protein production by gene expression. Recombinant nucleic acid molecules, for instance, have been engineered and incorporated into transgenic animals so that an expressed heterologous protein may be joined to a protein or peptide that allows secretion of the transgenic expression product into milk or urine, from which the protein may then be recovered. These procedures, however, may require lactating animals, with the attendant costs of maintaining individual animals or herds of large species, such as cows, sheep, or goats.

Historically, transgenic animals have been produced almost exclusively by microinjection of the fertilized egg. The pronuclei of fertilized eggs are microinjected in vitro with foreign, i.e., xenogeneic or allogeneic, heterologous DNA or hybrid DNA molecules. The microinjected fertilized eggs are then transferred to the genital tract of a pseudopregnant female (e.g., Krimpenfort et al., U.S. Pat. No. 5,175,384).

One system that holds potential is the avian reproductive system. The production of an avian egg begins with formation of a large yolk in the ovary of the hen. The unfertilized oocyte or ovum is positioned on top of the yolk sac. After ovulation, the ovum passes into the infundibulum of the oviduct where it is fertilized if sperm are present, and then moves into the magnum of the oviduct, which is lined with tubular gland cells. These cells secrete the egg-white proteins, including ovalbumin, lysozyme, ovomucoid, conalbumin and ovomucin, into the lumen of the magnum where they are deposited onto the avian embryo and yolk. The hen oviduct offers outstanding potential as a protein bioreactor because of the high levels of protein production, the promise of proper folding and post-translation modification of the target protein, the ease of product recovery, and the shorter developmental period of chickens compared to other potential animal species.

One method for creating permanent genomic modification of a eukaryotic cell is to integrate an introduced DNA into an existing chromosome. Only retroviruses have so far provided efficient integration. However, retroviral integration is directed to a number, albeit limited, of insertion sites within the recipient genome so that positional variation in heterologous gene expression can be evident. Unpredictability as to which insertion site is targeted introduces an undesirable lack of control over the procedure. An additional limitation of the use of retroviruses is that the size of the nucleic acid molecule encoding the virus and heterologous sequences is restricted to about 8 kb. Although wild-type adeno-associated virus (AAV) often integrates at a specific region in the human genome, vectors derived from AAV do not integrate site-specifically due to the deletion of the toxic rep gene. Other well-known methods for genomic modification of animal cells include transfection of DNA using calcium phosphate co-precipitation, electroporation, lipofection, microinjection, protoplast fusion and particle bombardment, all of which methods typically produce random integration and at low frequency. Homologous recombination produces site-specific integration, but the frequency of such integration usually is very low.

An alternative method that has been considered for driving the integration of heterologous nucleic acid fragments into a chromosome is the use of a site-specific recombinase (integrase) that can catalyze the insertion or excision of nucleic acid fragments. These enzymes recognize relatively short unique nucleic acid sequences that serve for both recognition and recombination. Examples include Cre (Sternberg & Hamilton, 1981, J. Mol. Biol. 150: 467-486, 1981), Flp (Broach et al., 1982, Cell 29: 227-234, 1982) and R (Matsuzaki et al., 1990, J. Bact. 172:610-618, 1990).

A novel class of phage integrases that includes the integrase from the phage phiC31 can mediate highly efficient integration of transgenes in mammalian cells both in vitro and in vivo (Thyagarajan et al., Mol. Cell Biol. 21: 3926-3934 (2001)). Constructs and methods of using recombinase to integrate heterologous DNA into a plant, insect or mammalian genome are described by Calos in U.S. Pat. Ser. No. 6,632,672.

The phiC31 integrase is a member of a subclass of integrases, termed serine recombinases, that include R4 and TP901-1. Unlike the phage lambda integrases, which belong to a tyrosine class of recombinases, the serine integrases do not require cofactors such as integration host factor. The phiC31 integrase normally mediates integration of the phiC31 bacteriophage into the genome of Streptomyces via recombination between the attP recognition sequence of the phage genome and the attB recognition sequence within the bacterial genome. When a plasmid is equipped with a single attB site, phiC31 integrase will detect and mediate crossover between the attB site and a pseudo-attP site within the mammalian genome. Such pseudo-attP integration sites have now been identified in the mouse and human genomes. If the heterologous DNA is in a circular or supercoiled form, the entire plasmid becomes integrated with attL and attR arms flanking the nucleic acid insert. PhiC31 integrase is not able to mediate the integration into genomic DNA of sequences bearing attP sites.

PhiC31 integrase-mediated integration results in the destruction of the recognition or recombination sites themselves so that the integration reaction is irreversible. This will bypass the primary concern inherent with other recombinases, i.e., the reversibility of the integration reaction and excision of the inserted DNA.

It has been estimated that there are 50 to 100 pseudo-attP sites in mammalian genomes (mouse and human) and some sites are apparently preferred for integration over others. The chicken genome, however, is only about one-third the size of mammalian genomes, and it was unknown whether there would be a sufficient number of pseudo attP sites in the chicken genome to allow efficient integrase-mediated integration.

We have found that the phiC3 1 integrase is active in avian cells, increasing the rate of integration over that of a non-integrase-mediated integration. Furthermore, we have determined that the phiC3] integrase works well at both 37° Celsius and 41° Celsius, showing that it will function in the environment of a developing avian embryo.

A need still exists, however, for methods by which avian chromosomes can be permanently modified in an efficient and site-specific manner and the genetically transformed cells used to generate transgenic birds.

### SUMMARY OF THE INVENTION

Integration of a transgene into a defined chromosomal site is useful to improve the predictability of expression of the transgene, which is particularly advantageous when creating transgenic avians. Transgenesis by methods that randomly insert a transgene into an avian genome is often inefficient since the transgene may not be expressed at the desired levels or in desired tissues.

A novel class of phage integrases, and in particular the integrase from phage phiC31, can mediate the efficient integration of transgenes into target cells both in vitro and in vivo. When a plasmid is equipped with a single attB site, phiC31 integrase detects attP homologous sequences, termed pseudo-attP sites, in a target genome and mediates crossover between the attB site and a pseudo attP site.

The present invention provides novel methods and recombinant polynucleotide molecules for transfecting and integrating a heterologous nucleic acid molecule into the genome of an avian cell. The methods of the invention deliver to an avian cell population a first nucleic acid molecule that comprises a region encoding a bacterial recombination site. A source of integrase activity also delivered to the avian cell can be an integrase-encoding nucleic acid sequence and its associated promoter included in the first nucleic acid molecule or as a region of a second nucleic acid molecule that may be co-delivered with the polynucleotide molecule. Alternatively, integrase protein itself can be delivered directly to the target cell.

The recombinant nucleic acid molecules of the present invention may further comprise a heterologous nucleotide sequence operably linked to a promoter so that the heterologous nucleotide sequence, when integrated into the genome DNA of a recipient avian cell, can be expressed to yield a desired polypeptide. The nucleic acid molecule may also include a second transcription initiation site, such as an internal ribosome entry site (IRES), operably linked to a second heterologous polypeptide-encoding region desired to be expressed with the first polypeptide in the same cell.

The heterologous nucleic acid molecule of the present invention may include a cassette for the expression in a recipient avian cell of a desired heterologous polypeptide. Optionally, the nucleic acid molecules may further comprise a marker such as, but not limited to, a puromycin resistance gene, a luciferase gene, EGFP-encoding gene, and the like.

Once delivered to a recipient avian cell, the phiC31 integrase mediates recombination between the att site within the nucleic acid molecule and a bacteriophage attachment site within the genomic DNA of the avian cell. Both att sites are disrupted and the nucleic acid molecule, with partial att sequences at each end, is stably integrated into the genome attP site. The phiC31 integrase, by disrupting the att sites of the incoming nucleic acid and of the recipient site within the avian cell genome, precludes any subsequent reverse recombination event that would excise the integrated nucleic acid and reduce the overall efficiency of stable incorporation of the heterologous nucleic acid.

Following delivery of the nucleic acid molecule and a source of integrase activity into an avian cell population and integrase-mediated recombination, the cells may be returned to an embryo. Late stage blastodermal cells may be returned to a hard shell egg, which is resealed for incubation until hatching. Stage I embryos may be directly microinjected with the polynucleotide and source of integrase activity, isolated, transfected and returned to a stage I embryo which is reimplanted into a hen for further development. Alternatively, the transfected cells may be maintained in vitro culture.

The present invention further provides modified isolated avian or artificial chromosomes useful as vectors to shuttle transgenes or gene clusters into the avian genome. By delivery to the modified chromosome to an isolated recipient cell, the target cell, and progeny thereof, become trisomic. The additional or trisomic chromosome will not affect the subsequent development of the recipient cell and/or an embryo, nor interfere with the reproductive capacity of an adult bird developed from such cells or embryos. The chromosome will also be stable within chicken cells. The invention provides methods to isolate a population of chromosomes for delivery into chicken embryos or early cells.

The method comprises inserting a lac-operator sequence into an isolated chromosome and, optionally, inserting a desired transgene sequence within the same chromosome. The lac operator region is typically a concatamer of a plurality of lac operators for the binding of multiple lac repressor molecules. A recombinant DNA molecule is constructed that includes an identified region of the target chromosome, a recombination site such as attB or attP, and the lac-operator concatamer. The recombinant molecule is delivered to an avian cell, and homologous recombination will integrate the heterologous polynucleotide and the lac-operator concatamer into the targeted chromosome. A tag-polypeptide, such as the GPF-lac-repressor fusion protein, binds to the lac-operator sequence for identification and isolation of the genetically modified chromosome. The tagged mitotic chromosome can be isolated using, for instance, flow cytometry.

Another aspect of the present invention is an avian cell genetically modified with a transgene vector by the methods of the invention. For example, in one embodiment, the transformed cell can be a chicken early stage blastodermal cell or a genetically transformed cell line, including a sustainable cell line. The transfected cell may comprise a transgene stably integrated into the nuclear genome of the recipient cell, thereby replicating with the cell so that each progeny cell receives a copy of the transfected nucleic acid. A particularly useful cell line for the delivery and integration of a transgene comprises a heterologous attP site that can increase the efficiency of integration of a polynucleotide by phiC31 integrase and, optionally, a region for expressing the integrase.

Another aspect of the present invention is methods of expressing a heterologous polypeptide in an avian cell by stably transfecting a cell by using site-specific integrase-mediation and a recombinant nucleic acid molecule, as described above, and culturing the transfected cell under conditions suitable for expression of the heterologous polypeptide under the control of the avian transcriptional regulatory region.

Yet another aspect of the present invention concerns transgenic birds, such as chickens, comprising a recombinant nucleic acid molecule and which preferably (though optionally) express a heterologous gene in one or more cells in the animal. Embodiments of the methods for the production of a heterologous polypeptide by the avian tissue involve providing a suitable vector and introducing the vector into embryonic blastodermal cells together with an integrase, preferably phiC31 integrase, so that the vector can integrate into the avian genome. A subsequent step involves deriving a mature transgenic avian from the transgenic blastodermal cells by transferring the transgenic blastodermal cells to an embryo and allowing that embryo to develop fully, so that the cells become incorporated into the bird as the embryo is allowed to develop. An alternative is to transfer a transfected nucleus to an enucleated recipient cell which may then develop into a zygote and ultimately an adult bird. The resulting chick is then grown to maturity.

In various embodiments of the transgenic bird of the present invention, the expression of the transgene may be restricted to specific subsets of cells, tissues or developmental stages utilizing, for example, trans-acting factors acting on the transcriptional regulatory region operably linked to the polypeptide-encoding region of interest of the present invention and which control gene expression in the desired pattern. Tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

The invention can be used to express, in large yields and at low cost, a wide range of desired proteins including those used as human and animal pharmaceuticals, diagnostics, and livestock feed additives. Proteins such as growth hormones, cytokines, structural proteins and enzymes including human growth hormone, interferon, lysozyme, and β-casein are examples of proteins which are desirably expressed in the oviduct and deposited in eggs according to the invention.

The present invention includes methods of producing transgenic avians, for example, transgenic chickens, which employ the use of integrase, cationic polymers and/nuclear localization signals. The present invention also includes the transgenic avians produced by these methods and other methods disclosed herein. The invention also includes the eggs produced by the transgenic avians produced by these methods and other methods disclosed herein.

In one embodiment, the methods of the invention include introducing into an avian cell: 1) a nucleic acid comprising a transgene; 2) an integrase activity; and 3) a cationic polymer. Such methods provide for an increased efficiency of transgenic avian production relative to identical methods without the cationic polymer.

In another embodiment, the methods include introducing into an avian cell: 1) a nucleic acid comprising a transgene; 2) an integrase activity; and 3) and a nuclear localization signal. Such methods provide for an increased efficiency of transgenic avian production relative to identical methods without the nuclear localization signal.

In another embodiment, the methods include introducing into an avian cell: 1) a nucleic acid comprising a transgene; 2) an integrase activity; 3) a cationic polymer; and 4) a nuclear localization signal. Such methods provide for an increased efficiency of transgenic avian production relative to identical methods without the cationic polymer or without the nuclear localization signal.

In one embodiment, the avian cell is a cell of an avian embryo. For example, the avian cell may be a cell of an early stage embryo comprising a germinal disc. The avian cell may be, for example, a cell of a stage I avian embryo, a cell of a stage II avian embryo, a cell of a stage III avian embryo, a cell of a stage IV avian embryo, a cell of a stage V avian embryo, a cell of a stage VI avian embryo, a cell of a stage VII avian embryo, a cell of a stage VIII avian embryo, a cell of a stage IX avian embryo, a cell of a stage X avian embryo, a cell of a stage XI avian embryo or a cell of a stage XII avian embryo. In one particularly useful embodiment, the avian cell is a cell of a stage X avian embryo.

The methods provide for the introduction of nucleic acid into the avian cell by any suitable technique known to those of skill in the art. For example, the nucleic acid may be introduced into the avian cell by microinjecting, transfection, electroporation or lipofection. In one particularly useful embodiment, the introduction of the nucleic acid is done by microinjecting.

The nucleic acid which includes a transgene may be DNA or RNA or a combination of RNA and DNA. The nucleic acid may comprise a single strand or may comprise a double strand. The nucleic acid may be a linear nucleic acid or may be an open or closed circular nucleic acid and may be naturally occurring or synthetic.

Integrase activity may be introduced into the avian cell in any suitable form. In one embodiment, an integrase protein is introduced into the avian cell. In another embodiment, a nucleic acid encoding an integrase is introduced into the avian cell. The nucleic acid encoding the integrase may be double stranded DNA, single stranded DNA, double stranded RNA, single stranded RNA or a single or double stranded nucleic acid which includes both RNA and DNA. In one particularly useful embodiment, the nucleic acid is mRNA. Integrase activity may be introduced into the avian cell by any suitable technique. Suitable techniques included those described herein for introducing the nucleic acid encoding a transgene into an avian cell. In one useful embodiment, the integrase activity is introduced into the avian cell with the nucleic acid encoding the transgene. For example, the integrase activity may be introduced into the avian cell in a mixture with the nucleic acid encoding the transgene.

In one embodiment, a nuclear localization signal (NLS) is associated with the nucleic acid which includes a transgene. For example, the NLS may be associated with the nucleic acid by a chemical bond. Examples of chemical bonds by which an NLS may be associated with the nucleic acid include an ionic bond, a covalent bond, hydrogen bond and Van der Waal's force. In one particularly useful embodiment, the nucleic acid which includes a transgene is associated with an NLS by an ionic bond. NLS may be introduced into the avian cell by any suitable technique. Suitable techniques included those described herein for introducing the nucleic acid encoding a transgene into an avian cell. In one useful embodiment, the NLS is introduced into the avian cell with the nucleic acid encoding the transgene. For example, the NLS may be introduced into the avian cell while associated with the nucleic acid encoding the transgene.

Cationic polymers may be employed to facilitate the production of transgenic avians. For example, the cationic polymers may be employed in combination with integrase and/or NLS. Any suitable cationic polymer may be used. For example, and without limitation, one or more of polyethylenimine, polylysine, DEAE-dextran, starburst dendrimers and starburst polyamidoamine dendrimers may be used. In a particularly useful embodiment, the cationic polymer includes polyethylenimine. The cationic polymer may be introduced into the avian cell by any suitable technique. Suitable techniques included those described herein for introducing the nucleic acid encoding a transgene into an avian cell. In one useful embodiment, the cationic polymer is introduced into the avian cell in a mixture with the nucleic acid encoding the transgene. For example, the cationic polymer may be introduced into the avian cell while associated with the nucleic acid encoding the transgene.

In one particularly useful embodiment of the invention, the transgene includes a coding sequence which is expressed in a cell of the transgenic avian producing a peptide or a polypeptide (e.g., a protein). The coding sequence may be expressed in any or all of the cells of the transgenic avian. For example, the coding sequence may be expressed in the blood, the magnum and/or the sperm of the transgenic avian. In a particularly useful embodiment of the invention, the polypeptide is present in an egg, for example, in the egg white, produce by the transgenic avian.

The methods of the invention include introducing the avian cell into a recipient avian, for example, a hen, wherein the recipient avian produces an offspring which includes the transgene. The avian cell may be introduced into a recipient avian by any suitable technique.

The present invention also includes methods of dispersing nucleic acid in a cell, for example an avian cell (e.g., an avian embryo cell). These methods include introducing into a cell a nucleic acid and a dispersing agent, for example, a cationic polymer (e.g., polyethylenimine, polylysine, DEAE-dextran, starburst dendrimers and/or starburst polyamidoamine dendrimers) in an amount that will disperse the nucleic acid in a cell. In one embodiment, the methods include introducing an avian cell into a recipient avian wherein the recipient avian produces an offspring which includes the transgene,

In one embodiment, the nucleic acid includes a transgene. NLS or integrase activity may also be introduced into the cell.

Typically, the dispersing of the nucleic acid is a homogeneous dispersing.

Any combination of features described herein are included within the scope of the present invention provided that the features included in any such combination are not mutually inconsistent as will be apparent from the context, this specification, and the knowledge of one of ordinary skill in the art.

Additional objects and aspects of the present invention will become more apparent upon review of the detailed description set forth below when taken in conjunction with the accompanying figures, which are briefly described as follows.

### BRIEF DESCRIPTION OF THE FIGURES

FIG. 1 illustrates phage integrase-mediated integration. A plasmid vector bearing the transgene includes the attB recognition sequence for the phage integrase. The vector along with integrase-coding mRNA, a vector expressing the integrase, or the integrase protein itself, are delivered into cells or embryos. The integrase recognizes DNA sequences in the avian genome similar to attP sites, termed pseudo-attP, and mediates recombination between the attB and pseudo-attP sites, resulting in the permanent integration of the transgene into the avian genome.

FIG. 2 illustrates the persistent expression of luciferase from a nucleic acid molecule after phiC31 integrase-mediated integration into chicken cells.

FIG. 3 illustrates the results of a puromycin resistance assay to measure phiC31 integrase-mediated integration into chicken cells.

FIG. 4 illustrates phiC31 integrase-mediated integration into quail cells. Puromycin resistance vectors bearing attB sites were cotransfected with phiC31 integrase, or a control vector, into QT6 cells, a quail fibrosarcoma cell line. One day after transfection, puromycin was added. Puromycin resistant colonies were counted 12 days post-transfection.

FIGS. 5A and 5B illustrate that phiC31 integrase can facilitate multiple integrations per avian cell. A puromycin resistance vector bearing an attB site was cotransfected with an enhanced green fluorescent protein (EGFP) expression vector bearing an attB site, and a phiC31 integrase expression vector. After puromycin selection, many puromycin resistant colonies expressed EGFP in all of their cells. FIGS. 5A and 5B are the same field of view with EGFP illuminated with ultraviolet light (FIG. 5A) and puromycin resistant colonies photographed in visible light (FIG. 5B). In FIG. 5B, there are 4 puromycin resistant colonies, two of which are juxtaposed at the top. One of these colonies expressed EGFP.

FIG. 6 shows maps of the small vectors used for integrase assays.

FIG. 7 shows integrase promotes efficient integration of large transgenes in avian cells.

FIG. 8 shows maps of large vectors used for integrase assays.

FIG. 9 illustrates the nucleotide sequence of the integrase-expressing plasmid pCMV-31int (SEQ ID NO: 1).

FIG. 10 illustrates the nucleotide sequence of the plasmid pCMV-luc-attB (SEQ ID NO: 2).

FIG. 11 illustrates the nucleotide sequence of the plasmid pCMV-luc-attP (SEQ ID NO: 3).

FIG. 12 illustrates the nucleotide sequence of the plasmid pCMV-pur-attB (SEQ ID NO: 4).

FIG. 13 illustrates the nucleotide sequence of the plasmid pCMV-pur-attP (SEQ ID NO: 5).

FIG. 14 illustrates the nucleotide sequence of the plasmid pCMV-EGFP-attB (SEQ ID NO: 6).

FIG. 15 illustrates the nucleotide sequence of the plasmid p12.0-lys-LSPIPNMM-CMV-pur-attB (SEQ ID NO: 7).

FIG. 16 illustrates the nucleotide sequence of the plasmid pOMIFN-lns-CMV-pur-attB (SEQ ID NO: 8).

FIG. 17 illustrates the nucleotide sequence of the integrase-expressing plasmid pRSV-lnt (SEQ ID NO: 9).

FIG. 18 illustrates the nucleotide sequence of the plasmid pCR-XL-TOPO-CMV-pur-attB (SEQ ID NO: 10).

FIG. 19 illustrates the nucleotide sequence of the attP containing polynucleotide SEQ ID NO: 11.

FIG. 20 illustrates in schematic form the integration of a heterologous att recombination site into an isolated chromosome. The attB sequence is linked to selectable maker such as a puromycin expression cassette and is flanked by sequences found in the target site of the chromosome to be modified. The DNA is transfected into cells containing the chromosome and stable transfectants are selected by drug resistance. Site specific integration may be confirmed by several techniques including PCR.

FIG. 21 illustrates the persistent expression of luciferase from a nucleic acid molecule after phiC31 integrase-mediated integration into chicken cells bearing a wild-type attP sequence.

FIG. 22 illustrates the distribution of plasmid DNA in a stage I embryo.

FIG. 23 illustrates the distribution of plasmid DNA in a stage I embryo in the presence of low molecular weight polyethylenimine.

FIG. 24 illustrates the distribution of plasmid DNA in a stage I embryo in the presence of low molecular weight polyethylenimine.

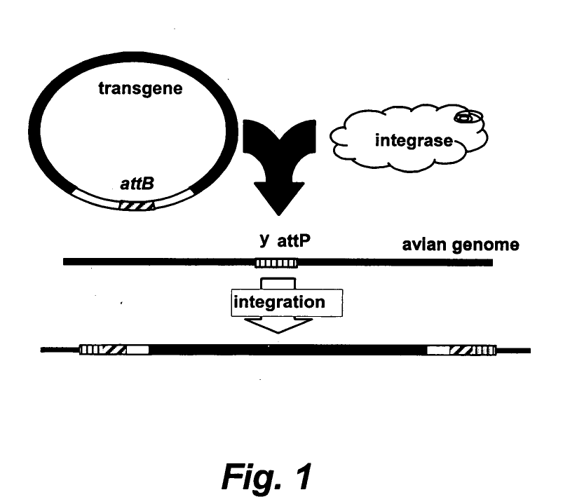

### DETAILED DESCRIPTION OF THE PREFERRED EMBODIMENTS

This description uses gene nomenclature accepted by the Cucurbit Genetics Cooperative as it appears in the Cucurbit Genetics Cooperative Report 18:85 (1995), which are incorporated herein by reference in its entirety. Using this gene nomenclature, genes are symbolized by italicized Roman letters. If a mutant gene is recessive to the normal type, then the symbol and name of the mutant gene appear in italicized lower case letters.

The disclosures of publications, patents, and published patent specifications referenced in this application are hereby incorporated by reference into the present disclosure to more fully describe the state of the art to which this invention pertains.

Definitions

For convenience, definitions of certain terms employed in the specification, examples, and appended claims are collected here.

As used in this specification and the appended claims, the singular forms “a,” “an” and “the” include plural references unless the content clearly dictates otherwise. Thus, for example, reference to “an antigen” includes a mixture of two or more such agents.

The term “avian” as used herein refers to any species, subspecies or race of organism of the taxonomic class ava, such as, but not limited to chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, New Hampshire, R.I., Australorp, Minorca, Amrox, Calif. Gray), as well as strains of turkeys, pheasants, quails, duck, ostriches and other poultry commonly bred in commercial quantities. It also includes an individual avian organism in all stages of development, including embryonic and fetal stages. The term “avian” also may denote “pertaining to a bird”, such as “an avian (bird) cell.”

The term “nucleic acid” as used herein includes any natural or synthetic linear and sequential array of nucleotides and nucleosides, for example cDNA, genomic DNA, mRNA, tRNA, oligonucleotides, oligonucleosides and derivatives thereof. For ease of discussion, such nucleic acids may be collectively referred to herein as “constructs,” “plasmids,” or “vectors.” The term “nucleic acid” further includes modified or derivatized nucleotides and nucleosides such as, but not limited to, halogenated nucleotides such as, but not only, 5-bromouracil, and derivatized nucleotides such as biotin-labeled nucleotides.

The terms “polynucleotide,” “oligonucleotide,” and “nucleic acid sequence” are used interchangeably herein and include, but are not limited to, coding sequences (polynucleotide(s) or nucleic acid sequence(s) which are transcribed and translated into polypeptide in vitro or in vivo when placed under the control of appropriate regulatory or control sequences); control sequences (e.g., translational start and stop codons, promoter sequences, ribosome binding sites, polyadenylation signals, transcription factor binding sites, transcription termination sequences, upstream and downstream regulatory domains, enhancers, silencers, and the like); and regulatory sequences (DNA sequences to which a transcription factor(s) binds and alters the activity of a gene's promoter either positively (induction) or negatively (repression)). No limitation as to length or to synthetic origin are suggested by the terms described above.

As used herein the terms “peptide,” “polypeptide” and “protein” refer to a polymer of amino acids in a serial array, linked through peptide bonds. A “peptide” typically is a polymer of at least two to about 30 amino acids linked in a serial array by peptide bonds. The term “polypeptide” includes proteins, protein fragments, protein analogues, oligopeptides and the like. The term “polypeptides” contemplates polypeptides as defined above that are encoded by nucleic acids, produced through recombinant technology (isolated from an appropriate source such as a bird), or synthesized. The term “polypeptides” further contemplates polypeptides as defined above that include chemically modified amino acids or amino acids covalently or noncovalently linked to labeling moieties.

The terms “percent sequence identity” or “percent sequence similarity” as used herein refer to the degree of sequence identity between two nucleic acid sequences or two amino acid sequences as determined using the algorithm of Karlin & Attschul, Proc. Natl. Acad. Sci. 87: 2264-2268 (1990), modified as in Karlin & Attschul, Proc. Natl. Acad. Sci. 90: 5873-5877 (1993). Such an algorithm is incorporated into the NBLAST and XBLAST programs of Attschul et al., 1990, T. Mol. Biol. Q15: 403-410. BLAST nucleotide searches are performed with the NBLAST program, score=100, word length=12, to obtain nucleotide sequences homologous to a nucleic acid molecule of the invention. BLAST protein searches are performed with the XBLAST program, score=50, word length=3, to obtain amino acid sequences homologous to a reference polypeptide. To obtain gapped alignments for comparison purposes, Gapped BLAST is utilized as described in Attschul et al., Nucl. Acids Res. 25: 3389-3402 (1997). When utilizing BLAST and Gapped BLAST programs, the default parameters of the respective programs (e.g. XBLAST and NBLAST) are used. Other algorithms, programs and default settings may also be suitable such as, but not only, the GCG-Sequence Analysis Package of the U.K. Human Genome Mapping Project Resource Centre that includes programs for nucleotide or amino acid sequence comparisons. Examples of preferred algorithms are FASTA and BESTFIT.

The terms “recombinant nucleic acid” and “recombinant DNA” as used herein refer to combinations of at least two nucleic acid sequences that are not naturally found in a eukaryotic or prokaryotic cell. The nucleic acid sequences may include, but are not limited to, nucleic acid vectors, gene expression regulatory elements, origins of replication, suitable gene sequences that when expressed confer antibiotic resistance, protein-encoding sequences and the like. The term “recombinant polypeptide” is meant to include a polypeptide produced by recombinant DNA techniques. A recombinant polypeptide may be distinct from a naturally occurring polypeptide either in its location, purity or structure. Generally, a recombinant polypeptide will be present in a cell in an amount different from that normally observed in nature.

The term “gene” or “genes” as used herein refers to nucleic acid sequences that encode genetic information for the synthesis of a whole RNA, a whole protein, or any portion of such whole RNA or whole protein. Genes that are not naturally part of a particular organism's genome are referred to as “foreign genes,” “heterologous genes” or “exogenous genes” and genes that are naturally a part of a particular organism's genome are referred to as “endogenous genes”. The term “gene product” refers to an RNA or protein that is encoded by the gene. “Endogenous gene products” are RNAs or proteins encoded by endogenous genes. “Heterologous gene products” are RNAs or proteins encoded by “foreign, heterologous or exogenous genes” and are, therefore, not naturally expressed in the cell.

The term “expressed” or “expression” as used herein refers to the transcription from a gene to give an RNA nucleic acid molecule at least complementary in part to a region of one of the two nucleic acid strands of the gene. The term “expressed” or “expression” as used herein may also refer to the translation from an RNA molecule to give a protein, a polypeptide or a portion thereof.

The term “operably linked” refers to an arrangement of elements wherein the components so described are configured so as to perform their usual function. Control sequences operably linked to a coding sequence are capable of effecting the expression of the coding sequence. The control sequences need not be contiguous with the coding sequence, so long as they function to direct the expression thereof. For example, intervening untranslated yet transcribed sequences can be present between a promoter sequence and the coding sequence and the promoter sequence can still be considered “operably linked” to the coding sequence.

The term “transcription regulatory sequences” as used herein refers to nucleotide sequences that are associated with a gene nucleic acid sequence and which regulate the transcriptional expression of the gene. Exemplary transcription regulatory sequences include enhancer elements, hormone response elements, steroid response elements, negative regulatory elements, and the like.

The term “promoter” as used herein refers to the DNA sequence that determines the site of transcription initiation by an RNA polymerase. A “promoter-proximal element” is a regulatory sequence generally within about 200 base pairs of the transcription start site.

The term “internal ribosome entry sites (IRES)” as used herein refers to a region of a nucleic acid, most typically an RNA molecule, wherein eukaryotic initiation of protein synthesis occurs far downstream of the 5′ end of the RNA molecule. A 43S pre-initiation complex comprising the elf2 protein bound to GTP and Met-tRNAᵢ^{Met}, the 40S ribosomal subunit, and factors elf3 and 31f1A may bind to an “IRES” before locating an AUG start codon. An “IRES” may be used to initiate translation of a second coding region downstream of a first coding region, wherein each coding region is expressed individually, but under the initial control of a single upstream promoter. An “IRES” may be located in a eukaryotic cellular mRNA.

The term “coding region” as used herein refers to a continuous linear arrangement of nucleotides which may be translated into a polypeptide. A full length coding region is translated into a full length protein; that is, a complete protein as would be translated in its natural state absent any post-translational modifications. A full length coding region may also include any leader protein sequence or any other region of the protein that may be excised naturally from the translated protein.

The terms “vector” or “nucleic acid vector” as used herein refer to a natural or synthetic single or double stranded plasmid or viral nucleic acid molecule (RNA or DNA) that can be transfected or transformed into cells and replicate independently of, or within, the host cell genome. The term “expression vector” as used herein refers to a nucleic acid vector that comprises a transcription regulatory region operably linked to a site wherein is, or can be, inserted, a nucleotide sequence to be transcribed and, optionally, to be expressed, for instance, but not limited to, a sequence coding at least one polypeptide.

The term “transfection” as used herein refers to the process of inserting a nucleic acid into a host cell. Many techniques are well known to those skilled in the art to facilitate transfection of a nucleic acid into an eukaryotic cell. These methods include, for instance, treating the cells with high concentrations of salt such as a calcium or magnesium salt, an electric field, detergent, or liposome mediated transfection, to render the host cell competent for the uptake of the nucleic acid molecules, and by such methods as micro-injection into a pro-nucleus, sperm-mediated and restriction-mediated integration.

The terms “recombinant cell” and “genetically transformed cell” refer to a cell comprising a combination of nucleic acid segments not found in a single cell with each other in nature. A new combination of nucleic acid segments can be introduced into an organism using a wide array of nucleic acid manipulation techniques available to those skilled in the art. The recombinant cell may harbor a vector that is extragenomic, i.e. that does not covalently insert into the cellular genome, including a non-nuclear (e.g. mitochondrial) genome(s). A recombinant cell may further harbor a vector or a portion thereof that is intragenomic, i.e. covalently incorporated within the genome of the recombinant cell.

As used herein, a “transgenic avian” is any avian, as defined above, including the chicken and quail, in which one or more of the cells of the avian contain heterologous nucleic acid introduced by manipulation, such as by transgenic techniques. The nucleic acid may be introduced into a cell, directly or indirectly, by introduction into a precursor of the cell by way of deliberate genetic manipulation, such as by microinjection or by infection with a recombinant virus. Genetic manipulation also includes classical cross-breeding, or in vitro fertilization. A recombinant DNA molecule may be integrated within a chromosome, or it may be extrachromosomally replicating DNA.

The terms “chimeric animal” or “mosaic animal” are used herein to refer to animals in which the recombinant gene is found, or in which the recombinant is expressed, in some but not all cells of the animal. The term “tissue-specific chimeric animal” indicates that the recombinant gene is present and/or expressed in some tissues but not others.

As used herein, the term “transgene” means a nucleic acid sequence that is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout).

The term “cytokine” as used herein refers to any secreted polypeptide that affects a function of cells and modulates an interaction between cells in the immune, inflammatory or hematopoietic response. A cytokine includes, but is not limited to, monokines and lymphokines. Examples of cytokines include, but are not limited to, interferon α2b, Interleukin-1 (IL-1), Interleukin-6 (IL-6), Interleukin-8 (IL-8), Tumor Necrosis Factor-□ (TNF-□) and Tumor Necrosis Factor n (TNF-□).

The term “antibody” as used herein refers to polyclonal and monoclonal antibodies and fragments thereof, and immunologic binding equivalents thereof. Antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-id) antibodies, and epitope-binding fragments of any of the above.

The term “immunoglobulin polypeptide” as used herein refers to a constituent polypeptide of an antibody or a polypeptide derived therefrom. An “immunological polypeptide” may be, but is not limited to, an immunological heavy or light chain and may include a variable region, a diversity region, joining region and a constant region or any combination, variant or truncated form thereof. The term “immunological polypeptides” further includes single-chain antibodies comprised of, but not limited to, an immunoglobulin heavy chain variable region, an immunoglobulin light chain variable region and optionally a peptide linker.

The terms “integrase” and “integrase activity” as used herein refer to a nucleic acid recombinase of the serine recombinase family of proteins.

The term “source of integrase activity” as used herein refers to a polypeptide or multimeric protein having serine recombinase (integrase) activity in an avian cell. The term may further refer to a polynucleotide encoding the serine recombinase, such as an mRNA, an expression vector, a gene or isolated gene that may be expressed as the recombinase-specific polypeptide or protein.

The term “recombination site” as used herein refers to a polynucleotide stretch comprising a recombination site normally recognized and used by an integrase. For example, λ phage is a temperate bacteriophage that infects E. coli. The phage has one attachment site for recombination (attP) and the E. coli bacterial genome has an attachment site for recombination (attB). Both of these sites are recombination sites for λ integrase. Recombination sites recognized by a particular integrase can be derived from a homologous system and associated with heterologous sequences, for example, the attP site can be placed in other systems to act as a substrate for the integrase.

The term “pseudo-recombination site” as used herein refers to a site at which an integrase can facilitate recombination even though the site may not have a sequence identical to the sequence of its wild-type recombination site. For example, a phiC31 integrase and vector carrying a phiC31 wild-type recombination site can be placed into an avian cell. The wild-type recombination sequence aligns itself with a sequence in the avian cell genome and the integrase facilitates a recombination event. When the sequence from the genomic site in the avian cell, where the integration of the vector took place, is examined, the sequence at the genomic site typically has some identity to, but may not be identical with, the wild-type bacterial genome recombination site. The recombination site in the avian cell genome is considered to be a pseudo-recombination site (e.g., a pseudo-attP site) at least because the avian cell is heterologous to the normal phiC31 phage/bacterial cell system. The size of the pseudo-recombination site can be determined through the use of a variety of methods including, but not limited to, (i) sequence alignment comparisons, (ii) secondary structural comparisons, (iii) deletion or point mutation analysis to find the functional limits of the pseudo-recombination site, and (iv) combinations of the foregoing.

A nucleic acid fragment of interest may be a trait-producing sequence, by which it is meant a sequence conferring a non-native trait upon the cell in which the protein encoded by the trait-producing sequence is expressed. The term “non-native” when used in the context of a trait-producing sequence means that the trait produced is different than one would find in an unmodified organism which can mean that the organism produces high amounts of a natural substance in comparison to an unmodified organism, or produces a non-natural substance. For example, the genome of a bird could be modified to produce proteins not normally produced in birds such as, for instance, human or mouse antibodies, human cytokines, etc. Other useful traits include disease resistance, meat flavor, animal size, and the like.

A nucleic acid fragment of interest may additionally be a “marker nucleic acid” or expressed as a “marker polypeptide”. Marker genes encode proteins that can be easily detected in transformed cells and are, therefore, useful in the study of those cells. Examples of suitable marker genes include β-galactosidase, green or yellow fluorescent proteins, enhanced green fluorescent protein, chloramphenicol acetyl transferase, luciferase, and the like. Such regions may also include those 5′ noncoding sequences involved with initiation of transcription and translation, such as the enhancer, TATA box, capping sequence, CAAT sequence, and the like

The term “transformed” as used herein refers to a heritable alteration in a cell resulting from the uptake of a heterologous DNA.

The term “trisomic” as used herein refers to a cell or animal, such as an avian cell or bird that has a 2n+1 chromosomal complement, where n is the haploid number of chromosomes, for the animal species concerned.

Techniques useful for isolating and characterizing the nucleic acids and proteins of the present invention are well known to those of skill in the art and standard molecular biology and biochemical manuals may be consulted to select suitable protocols without undue experimentation. See, for example, Sambrook et al, 1989, “Molecular Cloning: A Laboratory Manual”, 2nd ed., Cold Spring Harbor, the content of which is herein incorporated by reference in its entirety.

Abbreviations

Abbreviations used in the present specification include the following: aa, amino acid(s); bp, base pair(s); kb, kilobase; att, bacterial recombination attachment site; IU, infectious units.

In the standard method of integrase mediated-transgenesis, a serine recombinase integrase mediates recombination between an attB site on a transgene vector and a pseudo attP site on a chromosome. In the method of the invention for integrase-mediated transgenesis, a heterologous wild-type attP site can be integrated into an avian nuclear genome to create a transgenic cell line or bird. A serine recombinase (integrase) and an attB-bearing transgene vector are then introduced into cells harboring the heterologous attP site, or into embryos derived from birds which bear the attP recombination site. The locations of attP and attB may be reversed such that the attB site is inserted into an avian chromosome and the attP sequence resides in an incoming transgene vector. In either case, the att site of the introduced vector would then preferentially recombine with the integrated heterologous att site in the genome of the recipient cell.

The methods of the invention are based, in part, on the discovery that there exist in avian genomes a number of specific nucleic acid sequences, termed pseudo-recombination sites, the sequences of which may be distinct from wild-type recombination sites but which can be recognized by a site-specific integrase and used to promote the efficient insertion of heterologous genes or polynucleotides into the targeted avian nuclear genome. The inventors have identified pseudo-recombination sites in avian cells capable of recombining with a recombination site, such as an attB site within a recombinant nucleic acid molecule introduced into the target avian cell. The invention is also based on the prior integration of a heterologous att recombination site, typically isolated from a bacteriophage or a modification thereof, into the genome of the target avian cell.

Integration into a predicted chromosomal site is useful to improve the predictability of expression, which is particularly advantageous when creating transgenic avians. Transgenesis by methods that result in insertion of the transgene into random positions of the avian genome is unpredictable since the transgene may not express at the expected levels or in the predicted tissues.

The invention as disclosed herein, therefore, provides methods for site-specifically genetically transforming an avian nuclear genome. In general, an avian cell having a first recombination site in the nuclear genome is transformed with a site-specific polynucleotide construct comprising a second recombination sequence and one or more polynucleotides of interest. Into the same cell, integrase activity is introduced that specifically recognizes-the first and second recombination sites under conditions such that the polynucleotide sequence of interest is inserted into the nuclear genome via an integrase-mediated recombination event between the first and second recombination sites.

The integrase activity, or a source thereof, can be introduced into the avian cell prior to, or concurrent with, the introduction of the site-specific construct. The integrase can be delivered to a cell as a polypeptide, or by expressing the integrase from a source polynucleotide such as an mRNA or from an expression vector that encodes the integrase, either of which can be delivered to the target avian cell before, during or after delivery of the polynucleotide of interest. Any integrase that has activity in an avian cell may be useful in the present invention, including HK022 (Kolot et al., Biotechnol. Bioeng., 84: 56-60 (2003)). Preferably, the integrase is a serine recombinase as described, for example, by Smith & Thorpe, in Mol. Microbiol., 44: 299-307 (2002). More preferably, the integrase is a bacteriophage integrase such as, but not limited to, TP901-1 (Stoll et al., J. Bact., 184: 3657-3663 (2002); Olivares et al., Gene, 278:167-176 (2001). Most preferably, the integrase is from the phage phiC31.

The nucleotide sequence of the junctions between an integrated transgene into the attP (or attB site) would be known. Thus, a PCR assay can be designed by one of skill in the art to detect when the integration event has occurred. The PCR assay for integration into a heterologous wild-type attB or attP site can also be readily incorporated into a quantitative PCR assay using Taqman™ or related technology so that the efficiency of integration can be measured.

The minimal attB and attP sites able to catalyze recombination mediated by the phiC31 integrase are 34 and 39 bp, respectively. In cell lines that harbor a heterologous integrated attP site, however, integrase has a preference for the inserted attP over any pseudo-attP sites of similar length, because pseudo-attP sites have very low sequence identity (between 10 to 50% identity) compared to the more efficient wild-type attP sequence. It is within the scope of the methods of the invention, however, for the recombination site within the target avian genome to be a pseudo-att site such as a pseudo-attP site or an attP introduced into an avian genome.

The sites used for recognition and recombination of phage and bacterial DNAs (the native host system) are generally non-identical, although they typically have a common core region of nucleic acids. The bacterial sequence is generally called the attB sequence (bacterial attachment) and the phage sequence is called the attP sequence (phage attachment). Because they are different sequences, recombination will result in a stretch of nucleic acids (called attL or attR for left and right) that is neither an attB sequence or an attP sequence, and likely is functionally unrecognizable as a recombination site to the relevant enzyme, thus removing the possibility that the enzyme will catalyze a second recombination reaction that would reverse the first.

The integrase may recognize a recombination site where sequence of the 5′ region of the recombination site can differ from the sequence of the 3′ region of the recombination sequence. For example, for the phage phiC31 attP (the phage attachment site), the core region is 5′-TTG-3′ the flanking sequences on either side are represented here as attP5′ and attP3′, the structure of the attP recombination site is, accordingly, attP5′-TTG-attP3′. Correspondingly, for the native bacterial genomic target site (attB) the core region is 5′-TTG-3′, and the flanking sequences on either side are represented here as attB5′ and attB3′, the structure of the attB recombination site is, accordingly, attB5′-TTG-attB3′. After a single-site, phiC31 integrase-mediated recombination event takes place between the phiC31 phage and the bacterial genome, the result is the following recombination product: attB5′-TTG-attP3′{phiC31 vector sequences}attP5′-TTG-attB3′. In the method of invention, the attB site will be within a recombinant nucleic acid molecule that may be delivered to a target avian cell. The corresponding attP (or pseudo-attP) site will be within the avian cell nuclear genome. Consequently, after phiC31 integrase mediated recombination, the recombination product, the nuclear genome with the integrated heterologous polynucleotide will have the sequence attP5′-TTG-attB3′{heterologous polynucleotide}-attB5′-TTG-attP3′. Typically, after recombination the post-recombination recombination sites are no longer able to act as substrate for the phiC31 integrase. This results in stable integration with little or no integrase mediated excision.

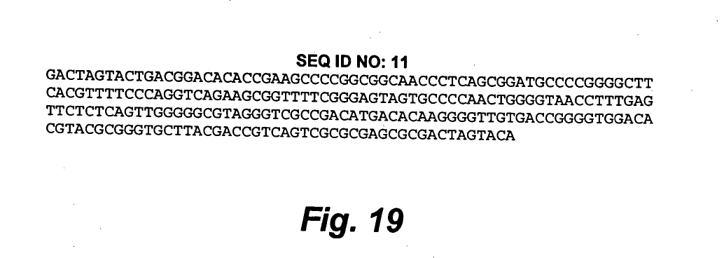

While the preferred recombination site to be included in the recombinant nucleic acid molecules and modified chromosomes of the present invention is the attP site, it is contemplated that any attP-like site may be used if compatible with the attB site. For instance, any pseudo-attP site of the chicken genome may be identified according to the methods of Example 7 below and used as a heterologous att recombination site. Such attP-like sites may have a sequence that is at least 25% identical to SEQ ID NO: 11 as shown in FIG. 19, such as described in Groth et al., Proc. Natl. Acad. Sci. U.S.A. 97: 5995-6000 (2000) incorporated herein by reference in its entirety. Preferably the selected site will have at least the same degree of efficiency of recombination as the attP site (SEQ ID NO: 11) itself.

In the methods of the present invention, the recipient avian cell population may be an isolated avian cell line such as, for example, DF-1 chicken fibroblasts, chicken DT40 cells or a cell population derived from an early stage embryo such as a chicken stage I or stage X embryo. A particularly useful avian cell population is blastodermal cells isolated from a stage X avian embryo. The methods of the present invention, therefore, include steps for the isolation of blastodermal cells that are then suspended in a cell culture medium or buffer for maintaining the cells in a viable state, and which allows the cell suspension to contact the nucleic acids of the present invention. It is also within the scope of the invention for the nucleic acid construct and the source of integrase activity to be delivered directly to an avian embryo such as a blastodermal layer, or to a tissue layer of an adult bird such as the lining of an oviduct.

When the recipient avian cell population is isolated from an early stage avian embryo, the embryos must first be isolated. For stage I avian embryos from, for example, a chicken, a fertilized ovum is surgically removed from a bird before the deposition of the outer hard shell has occurred. The nucleic acids for integrating a heterologous nucleic acid into a recipient avian cell genome may then be delivered to isolated embryos by lipofection, microinjection (as described in Example 6 below) or electroporation and the like. After delivery of the nucleic acid, the transfected embryo and its yolk may be deposited into the infundibulum of a recipient hen for the deposition of egg white proteins and a hard shell, and laying of the egg. Stage X avian embryos are obtained from freshly laid fertilized eggs and the blastodermal cells isolated as a suspension of cells in a medium, as described in Example 4 below. Isolated stage X blastodermal cell populations, once transfected, may be injected into recipient stage X embryos and the hard shell eggs resealed according to the methods described in U.S. Pat. No. 6,397,777.

In the methods of the invention, once a heterologous nucleic acid is delivered to the recipient avian cell, the integrase activity is expressed. The expressed integrase (or injected integrase polypeptide) then mediates recombination between the at site of the heterologous nucleic acid molecule, and the att (or pseudo att) site within the genomic DNA of the recipient avian cell.

It is within the scope of the present invention for the integrase-encoding sequence and a promoter operably linked thereto to be included in the delivered nucleic acid molecule and that expression of the integrase activity occurs before integration of the heterologous nucleic acid into the avian cell genome. Preferably, the integrase-encoding nucleic acid sequence and associated promoter are in an expression vector that may be co-delivered to the recipient avian cell with the heterologous nucleic acid molecule to be integrated into the recipient genome.

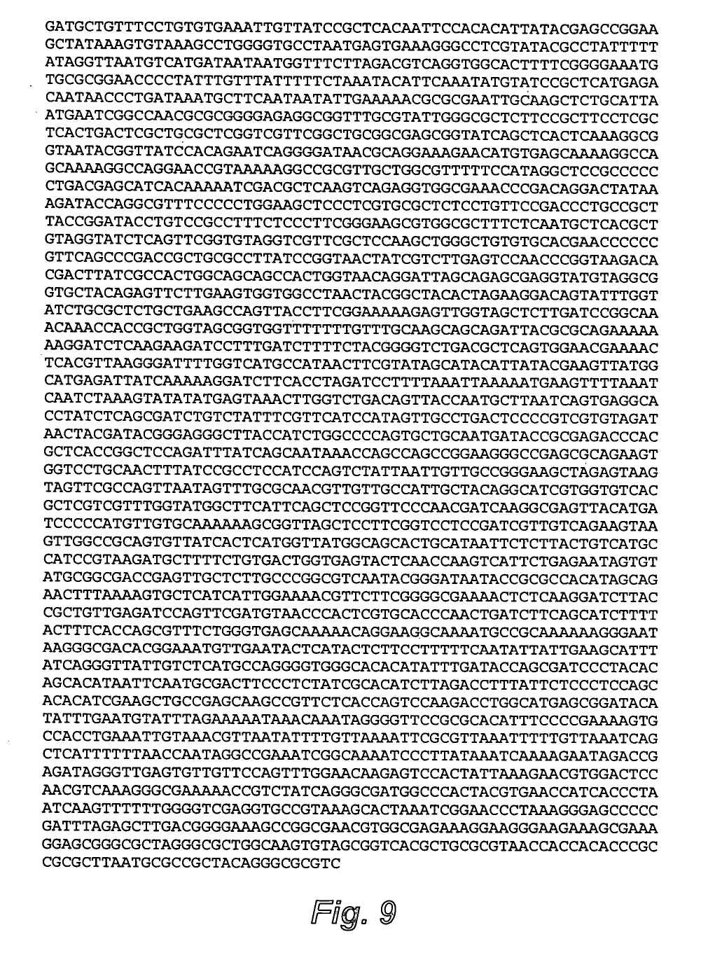

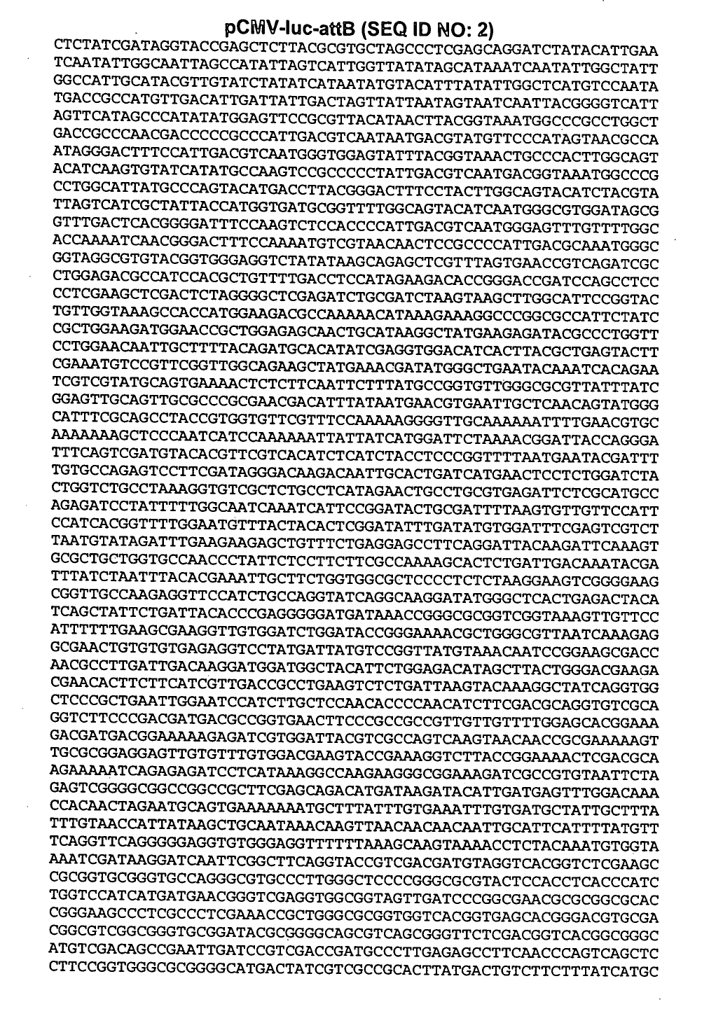

One suitable integrase expressing expression vector for use in the present invention is pCMV-C31int (SEQ ID NO: 1) as shown in FIG. 9, and described in Groth et al., Proc. Natl. Acad. Sci. U.S.A. 97: 5995-6000 (2000), incorporated herein by reference in its entirety. In pCMV-C31int, expression of the integrase-encoding sequence is driven by the CMV promoter. However, any promoter may be used that will give expression of the integrase in a recipient avian cell, including operably linked avian-specific gene expression control regions of the avian ovalbumin, lysozyme, ovomucin, ovomucoid gene loci, viral gene promoters, inducible promoters, the RSV promoter and the like.

The recombinant nucleic acid molecules of the present invention for delivery of a heterologous polynucleotide to the genome of a recipient avian cell may comprise a nucleotide sequence encoding the attB attachment site of Streptomyces ambofaciens as described in Thorpe & Smith, Proc. Natl. Acad. Sci. U.S.A. 95: 5505-5510 (1998). The nucleic acid molecule of the present invention further comprises an expression cassette for the expression in a recipient avian cell of a heterologous nucleic acid encoding a desired heterologous polypeptide. Optionally, the nucleic acid molecules may further comprise a marker such as, but not limited to, a puromycin resistance gene, a luciferase gene, EGFP, and the like.

It is contemplated that the expression cassette for introducing a desired heterologous polypeptide comprises a promoter operably linked to a nucleic acid encoding the desired polypeptide and, optionally, a polyadenylation signal sequence. Exemplary nucleic acids suitable for use in the present invention are more fully described in the examples below.

In the methods of the present invention, following delivery of the nucleic acid molecule and a source of integrase activity into an avian cell population, the cells are maintained under culture conditions suitable for the expression of the integrase and/or for the integrase to mediate recombination between the recombination site of the nucleic acid and recombination site in the genome of the recipient avian cell. When the recipient avian cell is cultured in vitro, such cells may be incubated at 37° Celsius if the cells are chicken early stage blastodermal cells. They may then be injected into an embryo within a hard shell, which is resealed for incubation until hatching. Alternatively, the transfected cells may be maintained in in vitro culture.

Site-Specific Nucleic Acid Constructs and Methods of Delivery to an Avian Cell

The present invention provides methods for the site-specific insertion of a heterologous nucleic acid molecule into the nuclear genome of an avian cell by delivering to a target avian cell that has a recombination site in its nuclear genome, a source of integrase activity, a site-specific construct that has another recombination site and a polynucleotide of interest, and allowing the integrase activity to facilitate a recombination event between the two recombination sites, thereby integrating the polynucleotide of interest into the avian nuclear genome.

(a) Expression vector nucleic acid molecules: A variety of recombinant nucleic acid expression vectors are suitable for use in the practice of the present invention. The site-specific constructs described herein can be constructed utilizing methodologies well known in the art of molecular biology (see, for example, Ausubel or Maniatis) in view of the teachings of the specification. As described above, the constructs are assembled by inserting into a suitable vector backbone a recombination site such as an attP or an attB site, a polynucleotide of interest operably linked to a gene expression control region of interest and, optionally a sequence encoding a positive selection marker. Polynucleotides of interest can include, but are not limited to, expression cassettes encoding a polypeptide to be expressed in the transformed avian cell or in a transgenic bird derived therefrom. The site-specific constructs are typically circular and may also contain selectable markers, an origin of replication, and other elements.

Any of the vectors of the present invention may also optionally include a sequence encoding a signal peptide that directs secretion of the polypeptide expressed by the vector from the transgenic cells, for instance, from tubular gland cells of the oviduct. This aspect of the invention effectively broadens the spectrum of exogenous proteins that may be deposited in the whites of avian eggs using the methods of the invention. Where an exogenous polypeptide would not otherwise be secreted, the vector bearing the coding sequence can be modified to comprise, for instance, about 60 bp encoding a signal peptide. The DNA sequence encoding the signal peptide is inserted in the vector such that the signal peptide is located at the N-terminus of the polypeptide encoded by the vector.

The expression vectors of the present invention can comprise an avian transcriptional regulatory region for directing expression of either fusion or non-fusion proteins. With fusion vectors, a number of amino acids are usually added to the desired expressed target gene sequence such as, but not limited to, a polypeptide sequence for thioredoxin. A proteolytic cleavage site may further be introduced at a site between the target recombinant protein and the fusion sequence. Additionally, a region of amino acids such as a polymeric histidine region may be introduced to allow binding of the fusion protein to metallic ions such as nickel bonded to a solid support, for purification of the fusion protein. Once the fusion protein has been purified, the cleavage site allows the target recombinant protein to be separated from the fusion sequence. Enzymes suitable for use in cleaving the proteolytic cleavage site include, but are not limited to, Factor Xa and thrombin. Fusion expression vectors that may be useful in the present invention include pGex (Amrad Corp., Melbourne, Australia), pRIT5 (Pharmacia, Piscataway, N.J.) and pMAL (New England Biolabs, Beverly, Mass.), that fuse glutathione S-transferase, protein A, or maltose E binding protein, respectively, to a desired target recombinant protein.

Epitope tags are short peptide sequences that are recognized by epitope specific antibodies. A fusion protein comprising a recombinant protein and an epitope tag can be simply and easily purified using an antibody bound to a chromatography resin, for example. The presence of the epitope tag furthermore allows the recombinant protein to be detected in subsequent assays, such as Western blots, without having to produce an antibody specific for the recombinant protein itself. Examples of commonly used epitope tags include V5, glutathione-S-transferase (GST), hemaglutinin (HA), the peptide Phe-His-His-Thr-Thr, chitin binding domain, and the like.

Preferred gene expression control regions for use in avian cells include, but are not limited to, avian specific promoters such as the chicken lysozyme, ovalbumin, or ovomucoid promoters, and the like. Particularly useful are tissue-specific promoters such as avian oviduct promoters that allow for expression and delivery of a heterologous polypeptide to an egg white.

Viral promoters serve the same function as bacterial or eukaryotic promoters and either provide a specific RNA polymerase in trans (bacteriophage T7) or recruit cellular factors and RNA polymerase (SV40, RSV, CMV). Viral promoters may be preferred as they are generally particularly strong promoters. A preferred promoter for use in avian cells is the RSV promoter.

Selection markers are valuable elements in expression vectors as they provide a means to select for growth of only those cells that contain a vector. Common selectable marker genes include those for resistance to antibiotics such as ampicillin, puromycin, tetracycline, kanamycin, bleomycin, streptomycin, hygromycin, neomycin, ZEOCIN™, and the like.

Another element useful in an expression vector is an origin of replication. Replication origins are unique DNA segments that contain multiple short repeated sequences that are recognized by multimeric origin-binding proteins and that play a key role in assembling DNA replication enzymes at the origin site. Suitable origins of replication for use in expression vectors employed herein include E. coli oriC, colE1 plasmid origin, and the like.

A further useful element in an expression vector is a multiple cloning site or polylinker. Synthetic DNA encoding a series of restriction endonuclease recognition sites is inserted into a vector, for example, downstream of the promoter element. These sites are engineered for convenient cloning of DNA into the vector at a specific position.

Elements such as the foregoing can be combined to produce expression vectors suitable for use in the methods of the invention. Those of skill in the art will be able to select and combine the elements suitable for use in their particular system in view of the teachings of the present specification.

(b) Genetically modified avian and artificial chromosomes: The present invention further provides modified chromosomes, either isolated avian or artificial chromosomes, are useful vectors to shuttle transgenes or gene clusters into the avian genome. By delivering the modified or artificial chromosome to an isolated recipient cell, the target cell, and progeny thereof, become trisomic. Preferably, an additional or triosomic chromosome will not affect the subsequent development of the recipient cell and/or an embryo, nor interfere with the reproductive capacity of an adult bird developed from such cells or embryos. The chromosome also should be stable within chicken cells. An effective method is also required to isolate a population of chromosomes for delivery into chicken embryos or early cells.

A number of artificial chromosomes are useful in the methods of the invention, including, for instance, a human chromosome modified to work as an artificial chromosome in a heterologous species as described, for example, for mice (Tomizuka et al., Proc. Natl. Acad Sci. U.S.A. 97: 722-727 (2000); for cattle (Kuroiwa et al., Nat. Biotechnol. 20: 889-894 (2002); a mammalian artificial chromosome used in mice (Co et al., Chromosome Res. 8: 183-191 (2000).

Chickens that are trisomic for microchromosome 16 have been described (Miller et al., Proc. Natl. Acad. Sci. U.S.A. 93: 3958-3962 (1996); Muscarella et al., J. Cell Biol. 101: 1749-1756 (1985). In these cases, triploidy and trisomy occurred naturally, and illustrate that an extra copy of one or more of the chicken chromosomes is compatible with normal development and reproductive capacity.

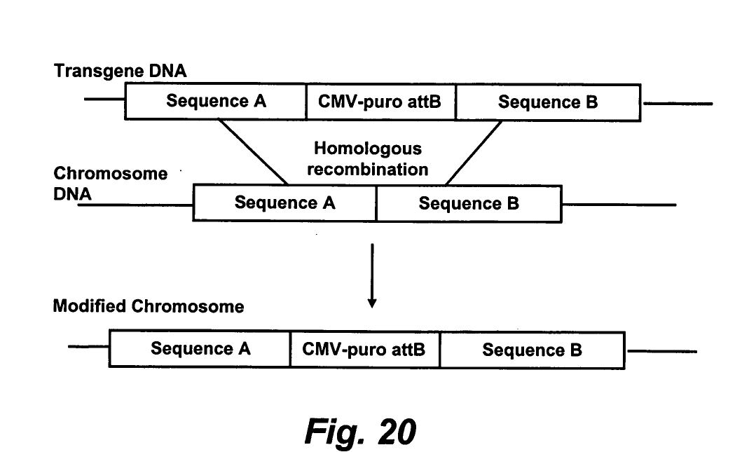

A useful chromosome isolation protocol can comprise the steps of inserting a lac-operator sequence (Robinett et al. J. Cell Biol. 135: 1685-1700 (1996) into an isolated chromosome and, optionally, inserting a desired transgene sequence within the same chromosome. Preferably, the lac operator region is a concatamer of a plurality of lac operators for the binding of multiple lac repressor molecules. Insertion can be accomplished, for instance, by identifying a region of known nucleotide sequence associated with a particular avian chromosome. A recombinant DNA molecule may be constructed that comprises the identified region, a recombination site such as attB or attP and a lac-operator concatamer. The recombinant molecule is delivered to an isolated avian cell, preferably, but not limited to, chicken DT40 cells that have elevated homologous recombination activity compared to other avian cell lines, whereupon homologous recombination will integrate the heterologous recombination site and the lac-operator concatamer into the targeted chromosome as shown in the schema illustrated in FIG. 20. A tag-polypeptide comprising a label domain and a lac repressor domain is also delivered to the cell, preferably by expression from a suitable expression vector. The nucleotide sequence coding for a GFP-lac-repressor fusion protein (Robinett et al., J. Cell Biol. 135: 1685-1700 (1996)) may be inserted into the same chromosome as the lac-operator insert. The lac repressor sequence, however, can also be within a different chromosome. An inducible promoter may also be used to allow the expression of the GFP-lac-repressor only after chromosome is to be isolated.

Induced expression of the GPF-lac-repressor fusion protein will result in specific binding of the tag fusion polypeptide to the lac-operator sequence for identification and isolation of the genetically modified chromosome. The tagged mitotic chromosome can be isolated using, for instance, flow cytometry as described in de Jong et al. Cytometry 35: 129-133 (1999) and Griffin et al. Cytogenet. Cell Genet. 87: 278-281 (1999).

A tagged chromosome can also be isolated using microcell technology requiring treatment of cells with the mitotic inhibitor colcemid to induce the formation of micronuclei containing intact isolated chromosomes within the cell. Final separation of the micronuclei is then accomplished by centrifugation in cytochalasin as described by Killary & Fournier in Methods Enzymol. 254: 133-152 (1995). Further purification of microcells containing only the desired tagged chromosome could be done by flow cytometry. It is contemplated, however, that alternative methods to isolate the mitotic chromosomes or microcells, including mechanical isolation or the use of laser scissors and tweezers, and the like.

Delivery of a Site-Specific Nucleic Acid to a Recipient Avian Cell or Embryo.

(a) Delivery of Polynucleotide Constructs.

Most non-viral methods of gene transfer rely on normal mechanisms used by eukaryotic cells for the uptake and intracellular transport of macromolecules. In preferred embodiments, non-viral gene delivery systems of the present invention rely on endocytic pathways for the uptake of the subject transcriptional regulatory region and operably linked polypeptide-encoding nucleic acid by the targeted cell. Exemplary gene delivery systems of this type include liposomal derived systems, poly-lysine conjugates, and artificial viral envelopes. Modified chromosomes as described above may be delivered to isolated avian embryonic cells for subsequent introduction to an embryo.

In a representative embodiment, a nucleic acid molecule can be entrapped in liposomes bearing positive charges on their surface (e.g., lipofectins) and (optionally) which are tagged with antibodies against cell surface antigens of the target tissue (Mizuno et al., 1992, NO Shinkei Geka 20: 547-551; PCT publication WO91/06309; Japanese patent application 1047381; and European patent publication EP-A-43075, all of which are incorporated herein by reference in their entireties).

In similar fashion, the gene delivery system can comprise an antibody or cell surface ligand that is cross-linked with a gene binding agent such as polylysine (see, for example, PCT publications WO093/04701, WO92/22635, WO92/20316, WO92/19749, and WO92/06180, all of which are incorporated herein by reference in their entireties). It will also be appreciated that effective delivery of the subject nucleic acid constructs via receptor-mediated endocytosis can be improved using agents which enhance escape of genes from the endosomal structures. For instance, whole adenovirus or fusogenic peptides of the influenza HA gene product can be used as part of the delivery system to induce efficient disruption of DNA-containing endosomes (Mulligan et al., 1993, Science 260-926; Wagner et al., 1992, Proc. Natl. Acad. Sci. 89:7934-7938; and Christiano et al., 1993, Proc. Natl. Acad. Sci. 90:2122-2126, all of which are incorporated herein by reference in their entireties). It is further contemplated that a recombinant nucleic acid molecule of the present invention may be delivered to a target host cell by other non-viral methods including by gene gun, microinjection, sperm-mediated transfer, or the like.

In yet another embodiment of the invention, an expression vector that comprises a heterologous attB recombination site and a region encoding a polypeptide deposited into an egg white are delivered to oviduct cells by in vivo electroporation. In this method, the luminal surface of an avian oviduct is surgically exposed. A buffered solution of the expression vector and a source of integrase activity such as a second expression vector expressing integrase (for example pCMV-int) is deposited on the luminal surface. Electroporation electrodes are then positioned on either side of the oviduct wall, the luminal electrode contacting the expression vector solution. After electroporation, the surgical incisions are closed. The electroporation will deliver the expression vectors to some, if not all, treated recipient oviduct cells to create a tissue-specific chimeric animal. Expression of the integrase allows for the integration of the heterologous polynucleotide into the genome of recipient oviduct cells. While this method may be used with any bird, a preferred recipient is a chicken due to the size of the oviduct. More preferred is a transgenic bird that has a transgenic attP recombinant site in the nuclear genomes of recipient oviduct cells, thus increasing the efficiency of integration of the expression vector.

The attB/P integrase system is preferred in the in vivo electroporation method to allow the formation of stable genetically transformed oviduct cells that otherwise progressively lose the heterologous expression vector.

The stably modified oviduct cells will express the heterologous polynucleotide and deposit the resulting polypeptide into the egg white of a laid egg. For this purpose, the expression vector will further comprise an oviduct-specific promoter such as ovalbumin or ovomucoid operably linked to the desired heterologous polynucleotide.

(b) Delivery of Chromosomes to Avian Cells.

Another aspect of the invention is the generation of a trisomic avian cell comprising a genetically modified extra chromosome. The extra chromosome may be an artificial chromosome or an isolated avian chromosome that has been genetically modified. Introduction of the extra chromosome to an avian cell will generate a trisomic cell with 2n+1 chromosomes, where n is the haploid number of chromosomes of a normal avian cell.

Delivery of an isolated chromosome into an isolated avian cell or embryo can be accomplished in several ways. Isolated mitotic chromosomes or a micronucleus containing an interphase chromosome can be injected into early stage I embryos by cytoplasmic injection. The injected zygote would then be surgically transferred to a recipient hen for the production and laying of a hard shell egg. This hard shell egg would then be incubated until hatching of a chick.

Isolated microcells can be fused to primordial germ cells (PGCs) isolated from the blood stream of late stage 15 embryos as described by Killary & Fournier in Methods Enzymol. 254: 133-152 (1995). The PGC/microcell hybrids can then be transplanted into the blood stream of a recipient embryo to produce gemiline chimeric chickens. (See Naito et al., Mol. Reprod. Dev. 39: 153-161 (1994)). The manipulated eggs would then incubated until hatching of the bird.

Blastodermal cells isolated from stage X embryos can be transfected with isolated mitotic chromosomes. Following in vitro transfection, the cells are transplanted back into stage X embryos as described, for example, in Etches et al., Poult. Sci., 72: 882-829 (1993), and the manipulated eggs are incubated to hatching.

Stage X blastodermal cells can also be fused with isolated microcells and then transplanted back into to stage X embryos or fused to somatic cells to be used as nuclear donors for nuclear transfer as described by Kuroiwa et al., Nat. Biotechnol. 20: 889-894 (2002).

Chromosomal vectors, as described above, may be delivered to a recipient avian cell by, for example, microinjection, liposomal delivery or microcell fusion.

In the methods of the invention, a site-specific integrase is introduced into an avian cell whose genome is to be modified. Methods of introducing functional proteins into cells are well known in the art. Introduction of purified integrase protein can ensure a transient presence of the protein and its activity. Thus, the lack of permanence associated with most expression vectors is not expected to be detrimental.

The integrase used in the practice of the present invention can be introduced into a target cell before, concurrently with, or after the introduction of a site-specific vector. The integrase can be directly introduced into a cell as a protein, for example, by using liposomes, coated particles, or microinjection, or into the blastodermal layer of an early stage avian embryo by microinjection. A source of the integrase can also be delivered to an avian cell by introducing to the cell an mRNA encoding the integrase and which can be expressed in the recipient cell as an integrase polypeptide. Alternately, a DNA molecule encoding the integrase can be introduced into the cell using a suitable expression vector.

The present invention provides novel nucleic acid vectors and methods of use that allow the phiC31 integrase to efficiently integrate a heterologous nucleic acid into an avian genome. A novel finding is that the phiC31 integrase is remarkably efficient in avian cells and increases the rate of integration of heterologous nucleic acid at least 30-fold over that of random integration. Furthermore, the phiC31 integrase works equally well at 37° C. and 41° C., indicating that it will function in the environment of the developing avian embryo, as shown in Example 1.

It is important to note that the present invention is not bound by any mechanism or theory of operation. For example, the mechanism by which integrase, or any other substance described herein, facilitates transgenesis is unimportant. Integrase, for example, may facilitate transgenesis by mediating the integration of DNA into the genome of a recipient cell or integrase may facilitate transgenesis by facilitating the entry of the DNA into the cell or integrase may facilitate transgenesis by some other mechanism.

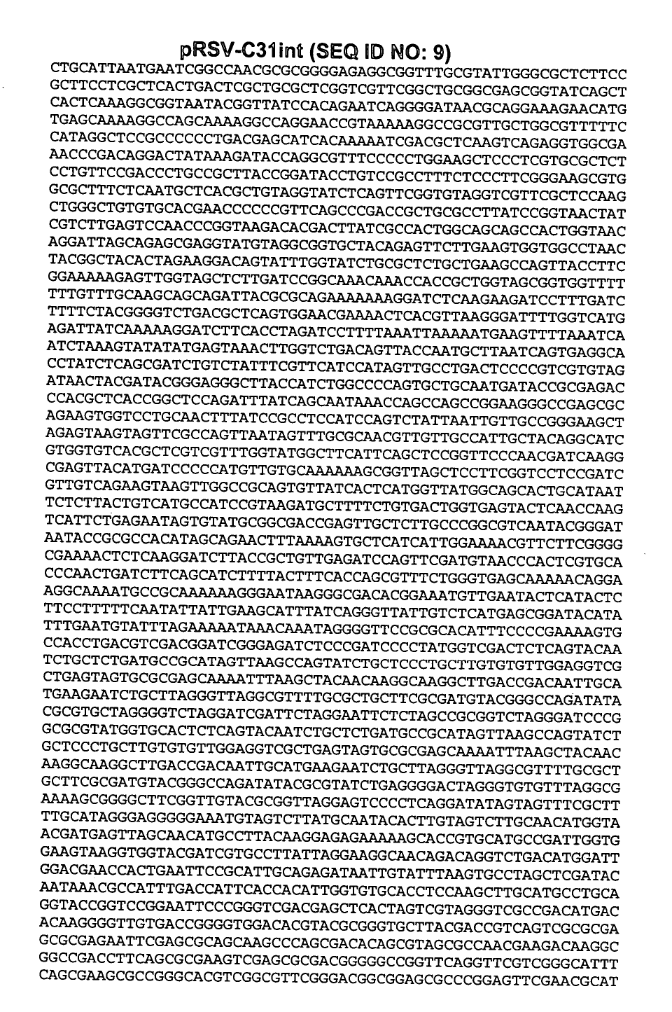

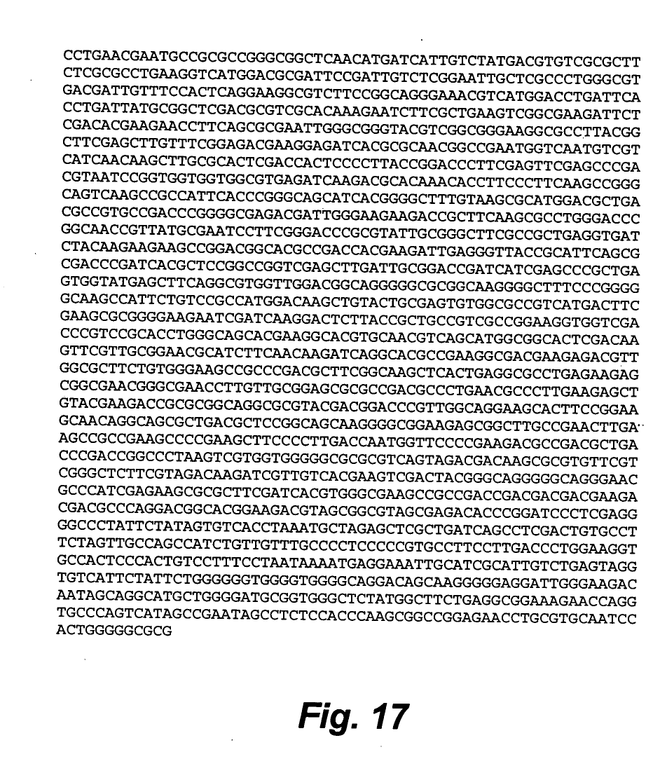

The site-specific vector components described above are useful in the construction of expression cassettes containing sequences encoding an integrase. One integrase-expressing vector useful in the methods of the invention is pCMV-C31int (SEQ ID NO: 1 as shown in FIG. 9) where the phiC31 integrase is encoded by a region under the expression control of the strong CMV promoter. Another preferred promoter generally useful in avian cells is the RSV promoter as used in SEQ ID NO: 9 shown in FIG. 17. Expression of the integrase is typically desired to be transient. Accordingly, vectors providing transient expression of the integrase are preferred. However, expression of the integrase can be regulated in other ways, for example, by placing the expression of the integrase under the control of a regulatable promoter (i.e., a promoter whose expression can be selectively induced or repressed).

Delivery of the nucleic acids introduced into avian cells, for example, embryonic avian cells, using methods of the invention may also be enhanced by mixing the nucleic acid to be introduced with a nuclear localization signal (NLS) peptide prior to introduction, for example, microinjection, of the nucleic acid. Nuclear localization signal (NLS) sequences are a class of short amino acid sequences which may be exploited for cellular import of linked cargo into a nucleus. The present invention envisions the use of any useful NLS peptide, including but not limited to, the NLS peptide of SV40 virus T-antigen.

An NLS of the invention is an amino acid sequence which mediates nuclear transport into the nucleus, wherein deletion of the NLS reduces transport into the nucleus. In certain embodiments, an NLS is a cationic peptide, for example, a highly cationic peptide. The present invention includes the use of any NLS sequence, including but not limited to, SV40 virus T-antigen. NLSs known in the art include, but are not limited to those discussed in Cokol et al., 2000, EMBO Reports, 1(5):411-415, Boulikas, T., 1993, Crit. Rev. Eukaryot. Gene Expr., 3:193-227, Collas, P. et al., 1996, Transgenic Research, 5: 451-458, Collas and Alestrom, 1997, Biochem. Cell Biol. 75: 633-640, Collas and Alestrom, 1998, Transgenic Research, 7: 303-309, Collas and Alestrom, Mol. Reprod. Devel., 1996, 45:431-438. The disclosure of each of these references is incorporated by reference herein in its entirety.

Not to be bound by any mechanism of operation, DNA is protected and hence stabilized by cationic polymers. The stability of DNA molecules in the cytoplasm of cells may be increased by mixing the DNA to be introduced, for example, microinjected with cationic polymers (for example, branched cationic polymers), such as polyethylenimine (PEI), polylysine, DEAE-dextran, starburst dendrimers, starburst polyamidoamine dendrimers, and other materials that package and condense the DNA molecules (Kukowska-Latallo et al., 1996, Proc. Natl. Acad. Sci. USA 93:4897-4902).

Once the DNA molecules are delivered to the cytoplasm of cells, they migrate into the cell's endocytotic vesicles. Furthermore, migration into the cell's endosome is followed by fast inactivation of DNA within the endolysosomal compartment in transfected or injected cells, both in vitro and in vivo (Godbey, W, et al. 1999, Proc Natl Acad Sci U S A 96: 5177-81; and Lechardeur, D, et al. 1999, Gene Ther 6: 482-97; and references cited therein). Accordingly, in certain embodiments, DNA uptake is enhanced by the receptor-mediated endocytosis pathway using transferrin-polylysine conjugates or adenoviral-mediated vesicle disruption to effect the release of DNA from endosomes. However, the invention is not limited to this or any other theory or mechanism of operation referred to herein.

Buffering the endosomal pH using endosomal-scaping elements also protects DNA from degradation (Kircheis, R, et al. 2001, Adv Drug Deliv Rev 53: 341-58; Boussif, O, et al. 1995, Proc Natl Acad Sci USA 92: 7297-301; and Pollard, H, et al. 1998, J Biol Chem 273: 7507-11; and references cited therein). Thus, in certain embodiments, DNA complexes are delivered with polycations or cationic polymers that possess substantial buffering capacity below physiological pH, such as polyethylenimine, lipopolyamines and polyamidoamine polymers. In certain embodiments, DNA condensing compounds, such as the ones described above, are combined with viruses (Curiel, D, et al. Proc Natl Acad Sci USA 88: 88504, 1991; Wagner, E, et al. Proc Natl Acad Sci USA 89: 6099-103, 1992 and Cotten, M, et al., 1992, Proc Natl Acad Sci U S A 89: 6094-8), viral peptides (Wagner, E, et al. 1992, Proc Natl Acad Sci U S A 89: 7934-8; Plank, C, et al. 1994, J Biol Chem 269: 12918-24) and subunits of toxins (Uherek, C, et al., 1998, J Biol Chem 273: 8835-48). These materials significantly enhance the release of DNA from endosomes. In certain embodiments, viruses, viral peptides, toxins or subunits of toxins may be coupled to DNA/polylysine complexes via biochemical means or specifically by a streptavidin-biotin bridge (Wagner et al., 1992, Proc. Natl. Acad. Sci. USA 89:6099-6103; Plank et al., 1994, J. Biol Chem. 269(17):12918-12924). In other certain embodiments, the virus that is complexed with the DNA may be adenovirus, retrovirus, vaccinia virus, or parvovirus. The viruses may be linked to PEI or another cationic polymer associated with the nucleic acid. In certain embodiments, the virus may be alphavirus, orthomyxovirus, or picornavirus. In certain embodiments, the virus is defective or chemically inactivated. The virus may be inactivated by short-wave UV radiation or the DNA intercalator psoralen plus long-wave UV. The adenovirus may coupled to polylysine, either enzymatically through the action of transglutaminase or biochemically by biotinylating adenovirus and streptavidinylating the polylysine moiety. Transferrin may also be useful in combination with cationic polymers, adenoviruses and/or other materials disclosed herein to produce transgenic avians. For example, DNA complexes containing PEI, PEI-modified transferrin, and PEI-bound influenza peptides may be used to enhance transgenic avian production.

In other certain embodiments, complexes containing plasmid DNA, transferrin-PEI conjugates, and PEI-conjugated peptides derived from the N-terminal sequence of the influenza virus hemagglutinin subunit HA-2 may be used to produce transgenic chickens. In certain embodiments, the PEI-conjugated peptide may be a an amino-terminal amino acid sequence of influenza virus hemagglutinin which may be elongated by an amphipathic helix or by carboxyl-terminal dimerization.

The present invention provides for methods of dispersing or distributing nucleic acid in a cell, for example, in an avian cell. The avian cell may be, for example, and without limitation, a cell of a stage I avian embryo, a cell of a stage II avian embryo, a cell of a stage III avian embryo, a cell of a stage IV avian embryo, a cell of a stage V avian embryo, a cell of a stage VI avian embryo, a cell of a stage VII avian embryo, a cell of a stage VIII avian embryo, a cell of a stage IX avian embryo, a cell of a stage X avian embryo, a cell of a stage XI avian embryo or a cell of a stage XII avian embryo. In one particularly useful embodiment, the avian cell is a cell of a stage X avian embryo.

In one aspect of the present invention, cationic polymers are useful to distribute, for example, homogeneously distribute, nucleic acid introduced into a cell, for example, an embryonic avian cell. The present invention contemplates the use of cationic polymers including, but not limited to, those disclosed herein.

However, substances other than cationic polymers also capable of distributing or dispersing nucleic acids in a cell are included within the scope of the present invention.

The concentration of cationic polymer used is not critical though, preferably, enough cationic polymer is present to coat the nucleic acid to be introduced into the avian cell. The cationic polymer may be present in an aqueous mixture with the nucleic acid to be introduced into the cell at a concentration in a range of an amount equal to about the weight of the nucleic acid to a concentration wherein the solution is saturated with cationic polymer. In one useful embodiment, the cationic polymer is present in an amount in a range of about 0.01% to about 50%, for example, about 0.1% to about 20% (e.g., about 5%). The molecular weights of the cationic polymers can range from a molecular weight of about 1,000 to a molecular weight of about 1,000,000. In one embodiment, the molecular weight of the cationic polymers range from about 5,000 to about 100,000 for example, about 20,000 to about 30,000.

In one particularly useful aspect of the invention, procedures that are effective to facilitate the production of a transgenic avian may be combined to provide for an enhanced production of a transgenic avian wherein the enhanced production is an improved production of a transgenic avian relative to the production of a transgenic avian by only one of the procedures employed in the combination. For example, one or more of integrase activity, NLS, cationic polymer or other technique useful to enhance transgenic avian production disclosed herein can be used in the same procedure to provide for an enhanced production of transgenic avians relative to an identical procedure which does not employ all of the same techniques useful to enhance transgenic avian production.

Transgenic Avian Cells.

Another aspect of the present invention is an avian cell genetically modified with a transgene vector according to the present invention and described above. For example, in one embodiment, the transformed cell can be a chicken early stage blastodermal cell or a genetically transformed cell line, including a sustainable cell line. The transfected cell according to the present invention may comprise a transgene stably integrated into the nuclear genome of the recipient cell, thereby replicating with the cell so that each progeny cell receives a copy of the transfected nucleic acid. A particularly useful cell line for the delivery and integration of a transgene comprises a heterologous attP site that can increase the efficiency of integration of a polynucleotide by phiC31 integrase and, optionally, a region for expressing the integrase.

A retroviral vector can be used to deliver the att site into the avian genome since an attP or attB site is less than 300 bp. For example, the attP site can be inserted into the NLB retroviral vector, which is based on the avian leukosis virus genome. A lentiviral vector is a particularly suitable vector because lentiviral vectors can transduce non-dividing cells, so that a higher percentage of cells will have an integrated attP site.

The lacZ region of NLB is replaced by the attP sequence. A producer cell line would be created by transformation of, for example, the Isolde cell line capable of producing a packaged recombinant NLB-attP virus pseudo-typed with the envA envelope protein. Supernatant from the Isolde NLB-attP line is concentrated by centrifugation to produce high titer preparations of the retroviral vector that can then be used to deliver the attP site to the genome of an avian cell, as described in Example 9 below.

An attP-containing line of transgenic birds are a source of attP transgenic embryos and embryonic cells. Fertile zygotes and oocytes bearing a heterologous attP site in either the maternal, paternal, or both, genomes can be used for transgenic insertion of a desired heterologous polynucleotide. A transgene vector bearing an attB site, for example, would be injected into the cytoplasm along with either an integrase expression plasmid, mRNA encoding the integrase or the purified integrase protein. The oocyte or zygote is then cultured to hatch by ex ovo methods or reintroduced into a recipient hen such that the hen lays a hard shell egg the next day containing the injected egg.

In another example, fertile stage VII-XII embryos hemizygous or homozygous for the heterologous attP sequence, are used as a source of blastodermal cells. The cells are harvested and then transfected with a transgene vector bearing an attB site along with a source of integrase. The transfected cells are then injected into the subgerminal cavity of windowed fertile eggs. The chicks that hatch will bear the transgene integrated into the attP site in a percentage of their somatic and germ cells. To obtain fully transgenic birds, chicks are raised to sexual maturity and those that are positive for the transgene in their semen are bred to non-transgenic mates.

In various embodiments, the genetically engineered cells of the invention may contain an integrase specifically recognizing recombination sites and which is introduced into genetically engineered cells containing a nucleic acid construct of the invention under conditions such that the nucleic acid sequence(s) of interest will be inserted into the nuclear genome. Methods for introducing such an integrase into a cell are described above.

In some embodiments, the site-specific integrase is introduced into the cell as a polypeptide. In alternative embodiments, the site-specific integrase is introduced into the transgenic cell as a polynucleotide encoding the integrase, such as an expression cassette optionally carried on a transient expression vector, and comprising a polynucleotide encoding the recombinase.

In one embodiment, the invention is directed to methods of using a vector for site-specific integration of a heterologous nucleotide sequence into the genome of an avian cell, the vector comprising a circular backbone vector, a polynucleotide of interest operably linked to a promoter, and a first recombination site, wherein the genome of the cell comprises a second recombination site and recombination between the first and second recombination sites is facilitated by phiC31 integrase. In certain embodiments, the integrase facilitates recombination between a bacterial genomic recombination site (attB) and a phage genomic recombination site (attP).

In another embodiment, the invention is directed to an avian cell having a transformed genome comprising an integrated heterologous polynucleotide of interest whose integration, mediated by phiC31 integrase, was into a recombination site native to the avian cell genome and the integration created a recombination-product site comprising the polynucleotide sequence. In yet another embodiment, integration of the polynucleotide was into a recombination site not native to the avian cell genome, but instead into a heterologous recombination site engineered into the avian cell genome.

In further embodiments, the invention is directed to transgenic birds comprising a modified cell and progeny thereof as described above, as well as methods of producing the same.

Cells genetically modified to carry a heterologous attB or attP site by the methods of the present invention can be maintained under conditions that, for example, keep them alive but do not promote growth, promote growth of the cells, and/or cause the cells to differentiate or dedifferentiate. Cell culture conditions may be permissive for the action of the integrase in the cells, although regulation of the activity of the integrase may also be modulated by culture conditions (e.g., raising or lowering the temperature at which the cells are cultured).

One aspect of the invention is a method for generating a genetically modified avian cell, and progeny thereof, using a tagged chromosome, the method comprising the steps of providing an isolated modified chromosome comprising a lac operator region and a first recombination site, delivering the modified chromosome to a avian cell, thereby generating a trisomic avian cell, delivering to the avian cell a source of a tagged polypeptide comprising a fluorescent domain and a lac repressor domain, delivering a source of integrase activity to the avian cell, delivering a polynucleotide comprising a second recombination site and a region encoding a polypeptide to the avian cell, maintaining the avian cell under conditions suitable for the integrase to mediate recombination between the first and second recombination sites, thereby integrating the polynucleotide into the modified chromosome and generating a genetically modified avian cell, expressing the tag polypeptide by the avian cell, allowing the tag polypeptide to bind to the modified chromosome so as to label the modified chromosome, and isolating the modified chromosome by selecting modified chromosomes having a tag polypeptide bound thereto.

In one embodiment of the invention, the second avian cell is selected from the group consisting of a stage VII-XII blastodermal cell, a stage I embryo, a stage X embryo; an isolated primordial germ cell, an isolated non-embryonic cell, and an oviduct cell.

In various embodiments, the isolated modified chromosome is an avian chromosome or an artificial chromosome.

In other embodiments of the invention, the step of providing an isolated modified chromosome comprising a lac operator region and a first recombination site comprises the steps of generating a trisomic avian cell by delivering to an isolated avian cell an isolated chromosome and a polynucleotide comprising a lac operator and a second recombination site, maintaining the trisomic cell under conditions whereby the heterologous polynucleotide is integrated into the chromosome by homologous recombination, delivering to the avian cell a source of a tag polypeptide to label the chromosome, and isolating the labeled chromosome.

In one embodiment of the invention, the lac operator region is a concatamer of lac operators. In other embodiments of the invention, the tag polypeptide is expressed from an expression vector.

In one embodiment of the invention, the tag polypeptide is microinjected into the cell. In various embodiments of the invention, the method of delivery of a chromosome to an avian cell is selected from the group consisting of liposome delivery, microinjection, microcell, electroporation and gene gun delivery, or a combination thereof.

In embodiments of the invention, the fluorescent domain of the tag polypeptide is GFP.

In another embodiment of the invention, the method further comprises the step of delivering the second avian cell to an avian embryo. The embryo may be maintained under conditions suitable for hatching as a chick.

In one embodiment of the invention, the second avian cell is maintained under conditions suitable for the proliferation of the cell, and progeny thereof.

In various embodiments of the invention, the source of integrase activity is delivered to a first avian cell as a polypeptide or expressed from a polynucleotide, said polynucleotide being selected from an mRNA and an expression vector.

In one embodiment of the invention, the tag polypeptide activity is delivered to the avian cell as a polypeptide or expressed from a polynucleotide operably linked to a promoter. In another embodiment of the invention, the promoter is an inducible promoter. In yet another embodiment of the invention, the integrase is phiC31 integrase and in various embodiments of the invention, the first and second recombination sites are selected from an attB and an attP site, but wherein the first and second sites are not identical.

Expression of Heterologous Proteins by Site-Specific Genetic Transformation of Avian Cells.

Another aspect of the present invention is a method of expressing a heterologous polypeptide in an avian cell by stably transfecting a cell by using site-specific integrase-mediation and a recombinant nucleic acid molecule, as described above, and culturing the transfected cell under conditions suitable for expression of the heterologous polypeptide under the control of the avian transcriptional regulatory region.

The protein of the present invention may be produced in purified form by any known conventional techniques. For example, chicken cells, an egg or an egg white may be homogenized and centrifuged. The supernatant may then be subjected to sequential ammonium sulfate precipitation and heat treatment. The fraction containing the protein of the present invention is subjected to gel filtration in an appropriately sized dextran or polyacrylamide column to separate the proteins. If necessary, the protein fraction may be further purified by HPLC or other methods well known in the art of protein purification.

The methods of the invention are useful for expressing nucleic acid sequences that are optimized for expression in avian cells and which encode desired polypeptides or derivatives and fragments thereof. Derivatives include, for instance, polypeptides with conservative amino acid replacements, that is, those within a family of amino acids that are related in their side chains (commonly known as acidic, basic, nonpolar, and uncharged polar amino acids). Phenylalanine, tryptophan, and tyrosine are sometimes classified jointly as aromatic amino acids and other groupings are known in the art (see, for example, “Biochemistry”, 2nd ed, L. Stryer, ed., W.H. Freeman & Co., 1981). Peptides in which more than one replacement has taken place can readily be tested for activity in the same manner as derivatives with a single replacement, using conventional polypeptide activity assays (e.g. for enzymatic or ligand binding activities).

Regarding codon optimization, if the recombinant nucleic acid molecules are transfected into a recipient chicken cell, the sequence of the nucleic acid insert to be expressed can be optimized for chicken codon usage. This may be determined from the codon usage of at least one, and preferably more than one, protein expressed in a chicken cell according to well known principles. For example, in the chicken the codon usage could be determined from the nucleic acid sequences encoding the proteins such as lysozyme, ovalbumin, ovomucin and ovotransferrin of chicken. Optimization of the sequence for codon usage can elevate the level of translation in avian eggs.

The present invention provides methods for the production of a protein by an avian cell comprising the steps of maintaining an avian cell, transfecting with a first expression vector and, optionally, a second expression vector, under conditions suitable for proliferation and/or gene expression and such that an integrase will mediate site specific recombination at att sites. The expression vectors may each have a transcription unit comprising a nucleotide sequence encoding a heterologous polypeptide, wherein one polypeptide is an integrase, a transcription promoter, and a transcriptional terminator. The cells may then be maintained under conditions for the expression and production of the desired heterologous polypeptide(s).

The present invention further relates to methods for gene expression by avian cells from nucleic acid vectors, and transgenes derived therefrom, that include more than one polypeptide-encoding region wherein, for example, a first polypeptide-encoding region can be operatively linked to an avian promoter and a second polypeptide-encoding region is operatively linked to an Internal Ribosome Entry Sequence (IRES). It is contemplated that the first polypeptide-encoding region, the IRES and the second polypeptide-encoding region of a recombinant DNA of the present invention may be arranged linearly, with the IRES operably positioned immediately 5′ of the second polypeptide-encoding region. This nucleic acid construct, when inserted into the genome of an avian cell or a bird and expressed therein, will generate individual polypeptides that may be post-translationally modified and combined in the white of a hard shell bird egg. Alternatively, the expressed polypeptides may be isolated from an avian egg and combined in vitro.

The invention, therefore, includes methods for producing multimeric proteins including immunoglobulins, such as antibodies, and antigen binding fragments thereof. Thus, in one embodiment of the present invention, the multimeric protein is an immunoglobulin, wherein the first and second heterologous polypeptides are immunoglobulin heavy and light chains respectively. Illustrative examples of this and other aspects of the present invention for the production of heterologous multimeric polypeptides in avian cells are fully disclosed in U.S. patent application Ser. No. 09/877,374, filed Jun. 8, 2001, by Rapp, published as US-2002-0108132-A1 on Aug. 8, 2002, and U.S. patent application Ser. No. 10/251,364, filed Sep. 18, 2002, by Rapp, both of which are incorporated herein by reference in their entirety.

Accordingly, the invention further provides immunoglobulin and other multimeric proteins that have been produced by transgenic avians of the invention.

In various embodiments, an immunoglobulin polypeptide encoded by the transcriptional unit of at least one expression vector may be an immunoglobulin heavy chain polypeptide comprising a variable region or a variant thereof, and may further comprise a D region, a J region, a C region, or a combination thereof. An immunoglobulin polypeptide encoded by an expression vector may also be an immunoglobulin light chain polypeptide comprising a variable region or a variant thereof, and may further comprise a J region and a C region. The present invention also contemplates multiple immunoglobulin regions that are derived from the same animal species, or a mixture of species including, but not only, human, mouse, rat, rabbit and chicken. In preferred embodiments, the antibodies are human or humanized.

In other embodiments, the immunoglobulin polypeptide encoded by at least one expression vector comprises an immunoglobulin heavy chain variable region, an immunoglobulin light chain variable region, and a linker peptide thereby forming a single-chain antibody capable of selectively binding an antigen.

Examples of therapeutic antibodies that may be produced in methods of the invention include but are not limited to HERCEPTIN™ (Trastuzumab) (Genentech, Calif.) which is a humanized anti-HER2 monoclonal antibody for the treatment of patients with metastatic breast cancer; REOPRO™ (abciximab) (Centocor) which is an anti-glycoprotein IIb/IIIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/Medimmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primate anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (CS) antibody (Alexion Pharm); D2E7 is a humanized anti-TNF-α antibody (CATIBASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

Production of Heterologous Protein by Transgenic Avians

One aspect of the present invention, therefore, concerns transgenic birds, such as chickens, comprising a recombinant nucleic acid molecule and which preferably (though optionally) express a heterologous gene in one or more cells in the animal. Suitable methods for the generation of transgenic avians having heterologous DNA incorporated therein are described, for example, in WO 99/19472 to Ivarie et al.; WO 00/11151 to Ivarie et al.; and WO 00/56932 to Harvey et al., all of which are incorporated herein by reference in their entirety.

Embodiments of the methods for the production of a heterologous polypeptide by the avian tissue such as the oviduct and the production of eggs which contain heterologous protein involve providing a suitable vector and introducing the vector into embryonic blastodermal cells together with an integrase, preferably phiC31 integrase, so that the vector can integrate into the avian genome. A subsequent step involves deriving a mature transgenic avian from the transgenic blastodermal cells produced in the previous steps. Deriving a mature transgenic avian from the blastodermal cells optionally involves transferring the transgenic blastodermal cells to an embryo and allowing that embryo to develop fully, so that the cells become incorporated into the bird as the embryo is allowed to develop. Another alternative is to transfer a transfected nucleus to an enucleated recipient cell which may then develop into a zygote and ultimately an adult bird. The resulting chick is then grown to maturity.

In an alternative embodiment, the cells of a blastodermal embryo are transfected or transduced with the vector and integrase directly within the embryo. It is contemplated, for example, that the recombinant nucleic acid molecules of the present invention may be introduced into a blastodermal embryo by direct microinjection of the DNA into a stage X or earlier embryo that has been removed from the oviduct. The egg is then returned to the bird for egg white deposition, shell development and laying. The resulting embryo is allowed to develop and hatch, and the chick allowed to mature.

In one embodiment, a transgenic bird of the present invention is produced by introducing into embryonic cells such as, for instance, isolated avian blastodermal cells, a nucleic acid construct comprising an attB recombination site capable of recombining with a pseudo-attP recombination site found within the nuclear genome of the organism from which the cell was derived, and a nucleic acid fragment of interest, in a manner such that the nucleic acid fragment of interest is stably integrated into the nuclear genome of germ line cells of a mature bird and is inherited in normal Mendelian fashion. It is also within the scope of the invention that the targeted cells for receiving the transgene have been engineered to have a heterologous attP recombination site integrated into the nuclear genome of the cells, thereby increasing the efficiency of recognition and recombination with a heterologous attB site.

In either case, the transgenic bird produced from the transgenic blastodermal cells is known as a “founder” Some founders can be chimeric or mosaic birds if, for example, microinjection does not deliver nucleic acid molecules to all of the blastodermal cells of an embryo. Some founders will carry the transgene in the tubular gland cells in the magnum of their oviducts and will express the heterologous protein encoded by the transgene in their oviducts. If the heterologous protein contains the appropriate signal sequences, it will be secreted into the lumen of the oviduct and onto the yolk of an egg.

Some founders are germ-line founders. A germ-line founder is a founder that carries the transgene in genetic material of its germ-line tissue, and may also carry the transgene in oviduct magnum tubular gland cells that express the heterologous protein. Therefore, in accordance with the invention, the transgenic bird will have tubular gland cells expressing the heterologous protein and the offspring of the transgenic bird will also have oviduct magnum tubular gland cells that express the selected heterologous protein. (Alternatively, the offspring express a phenotype determined by expression of the exogenous gene in a specific tissue of the avian.)

The invention can be used to express, in large yields and at low cost, a wide range of desired proteins including those used as human and animal pharmaceuticals, diagnostics, and livestock feed additives. Proteins such as growth hormones, cytokines, structural proteins and enzymes including human growth hormone, interferon, lysozyme, and β-casein are examples of proteins which are desirably expressed in the oviduct and deposited in eggs according to the invention. Other possible proteins to be produced include, but are not limited to, albumin, α-1 antitrypsin, antithrombin III, collagen, factors VIII, IX, X (and the like), fibrinogen, hyaluronic acid, insulin, lactoferrin, protein C, erythropoietin (EPO), granulocyte colony-stimulating factor (G-CSF), granulocyte macrophage colony-stimulating factor (GM-CSF), tissue-type plasminogen activator (tPA), feed additive enzymes, somatotropin, and chymotrypsin. Immunoglobulins (shown, for example in Example 10 below) and genetically engineered antibodies, including immunotoxins which bind to surface antigens on human tumor cells and destroy them, can also be expressed for use as pharmaceuticals or diagnostics.

In various embodiments of the transgenic bird of the present invention, the expression of the transgene may be restricted to specific subsets of cells, tissues or developmental stages utilizing, for example, trans-acting factors acting on the transcriptional regulatory region operably linked to the polypeptide-encoding region of interest of the present invention and which control gene expression in the desired pattern. Tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

The stably modified oviduct cells will express the heterologous polynucleotide and deposit the resulting polypeptide into the egg white of a laid egg. For this purpose, the expression vector will further comprise an oviduct-specific promoter such as ovalbumin or ovomucoid operably linked to the desired heterologous polynucleotide.

Another aspect of the present invention provides a method for the production in an avian of an heterologous protein capable of forming an antibody suitable for selectively binding an antigen. This method comprises a step of producing a transgenic avian incorporating at least one transgene, the transgene encoding at least one heterologous polypeptide selected from an immunoglobulin heavy chain variable region, an immunoglobulin heavy chain comprising a variable region and a constant region, an immunoglobulin light chain variable region, an immunoglobulin light chain comprising a variable region and a constant region, and a single-chain antibody comprising two peptide-linked immunoglobulin variable regions.

In one embodiment of this method, the isolated heterologous protein is an antibody capable of selectively binding to an antigen and which may be generated by combining at least one immunoglobulin heavy chain variable region and at least one immunoglobulin light chain variable region, preferably cross-linked by at least one disulfide bridge. The combination of the two variable regions generates a binding site that binds an antigen using methods for antibody reconstitution that are well known in the art.

The present invention also encompasses immunoglobulin heavy and light chains, or variants or derivatives thereof, to be expressed in separate transgenic avians, and thereafter isolated from separate media including serum or eggs, each isolate comprising one or more distinct species of immunoglobulin polypeptide. The method may further comprise the step of combining a plurality of isolated heterologous immunoglobulin polypeptides, thereby producing an antibody capable of selectively binding to an antigen. In this embodiment, for instance, two or more individual transgenic avians may be generated wherein one transgenic produces serum or eggs having an immunoglobulin heavy chain variable region, or a polypeptide comprising such, expressed therein. A second transgenic animal, having a second transgene, produces serum or eggs having an immunoglobulin light chain variable region, or a polypeptide comprising such, expressed therein. The polypeptides from two or more transgenic animals may be isolated from their respective sera and eggs and combined in vitro to generate a binding site capable of binding an antigen.

The present invention is further illustrated by the following examples, which are provided by way of illustration and should not be construed as limiting. The contents of all references, published patents and patents cited throughout the present application are hereby incorporated by reference in their entireties.

It will be apparent to those skilled in the art that various modifications, combinations, additions, deletions and variations can be made in the present invention without departing from the scope or spirit of the invention. For instance, features illustrated or described as part of one embodiment can be used in another embodiment to yield a still further embodiment. It is intended that the present invention covers such modifications, combinations, additions, deletions and variations as come within the scope of the appended claims and their equivalents.

### EXAMPLE 1

### Phage phiC31 Integrase Functions in Avian Cells.

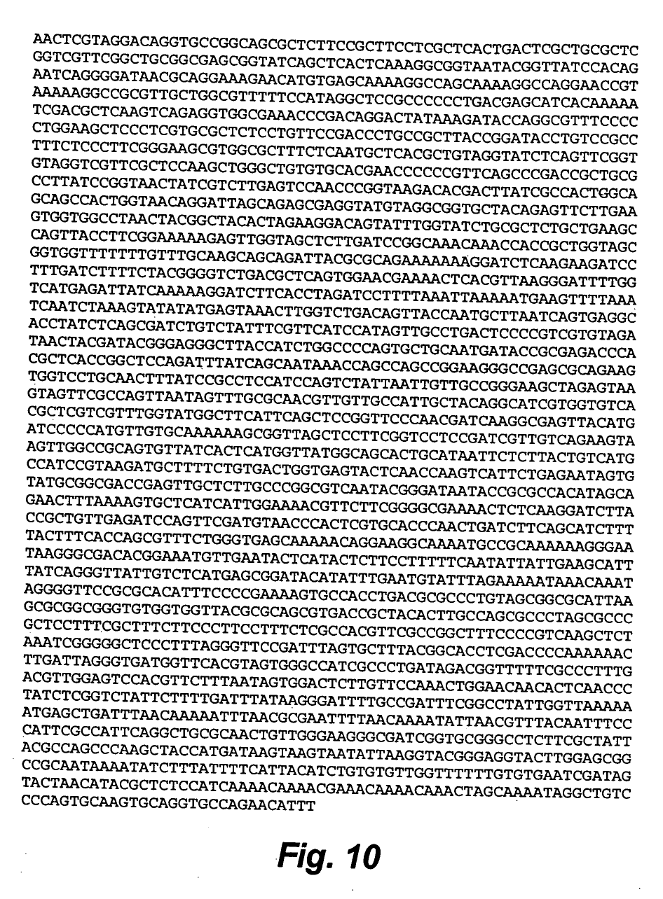

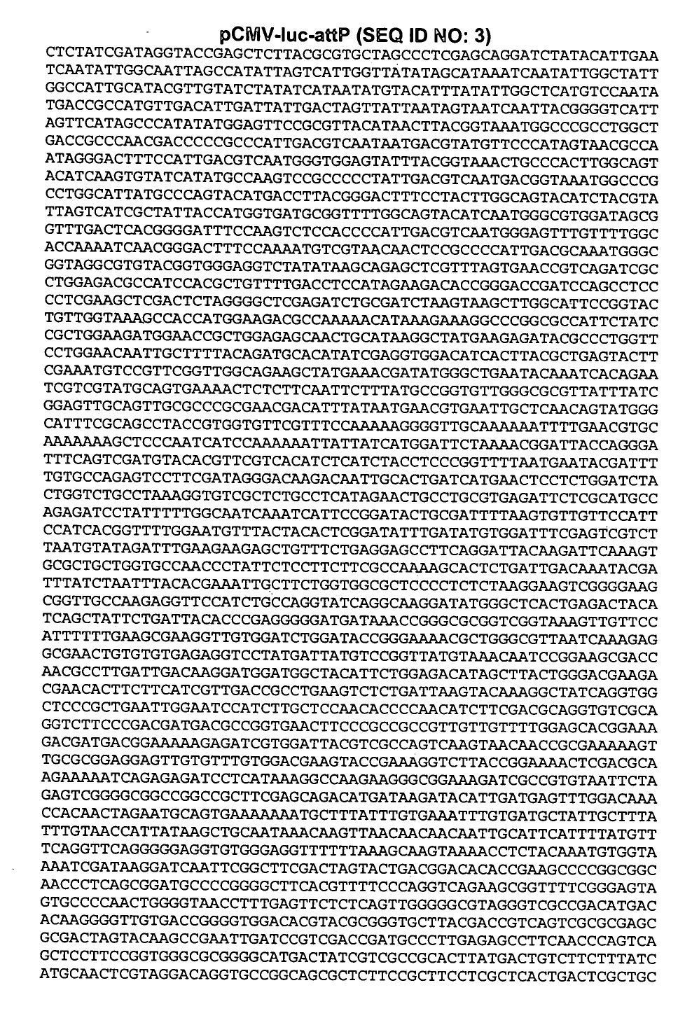

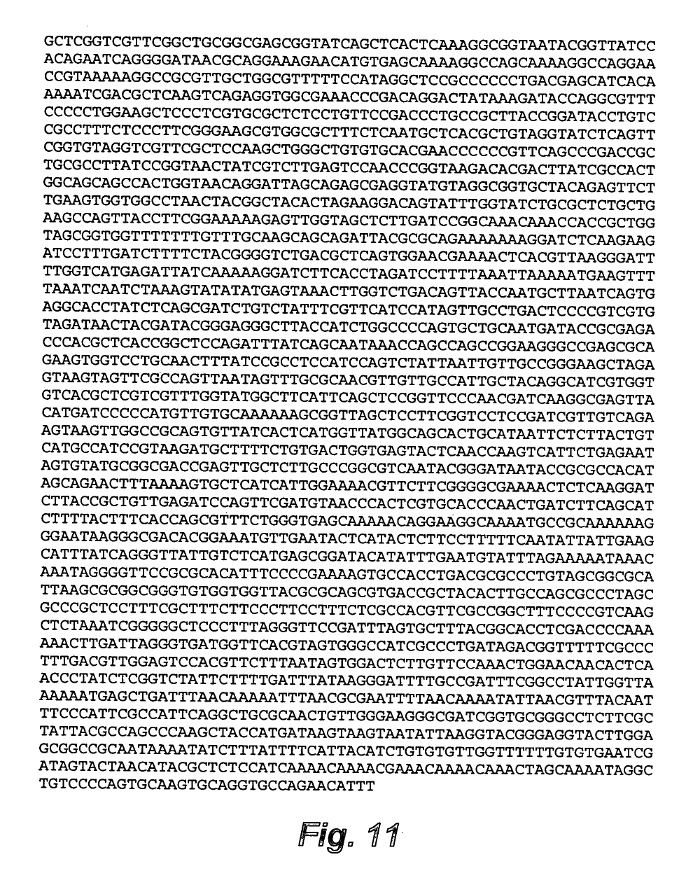

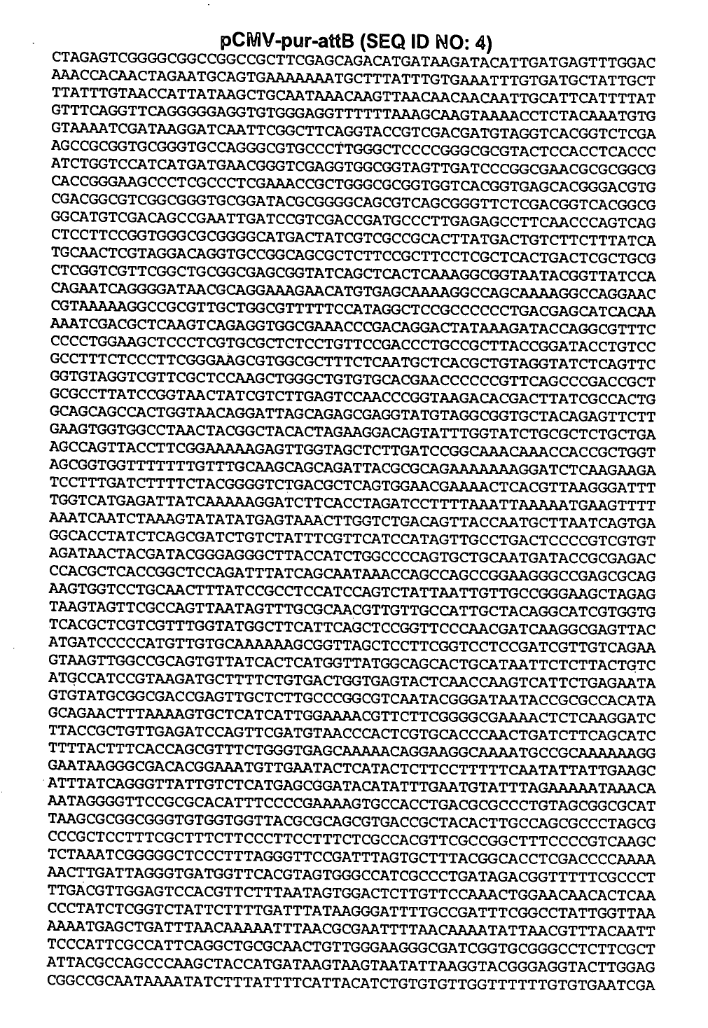

(a) A luciferase vector bearing either an attB (SEQ ID NO: 2 shown in FIG. 10) or attP (SEQ ID NO: 3 shown in FIG. 11) site was co-transfected with an integrase expression vector CMV-C31int (SEQ ID NO: 1) into DF-1 cells, a chicken fibroblast cell line. The cells were passaged several times and the luciferase levels were assayed at each passage.

Cells were passaged every 34 days and one third of the cells were harvested and assayed for luciferase. The expression of luciferase was plotted as a percentage of the expression measured 4 days after transfection. A luciferase expression vector bearing an attP site as a control was also included.

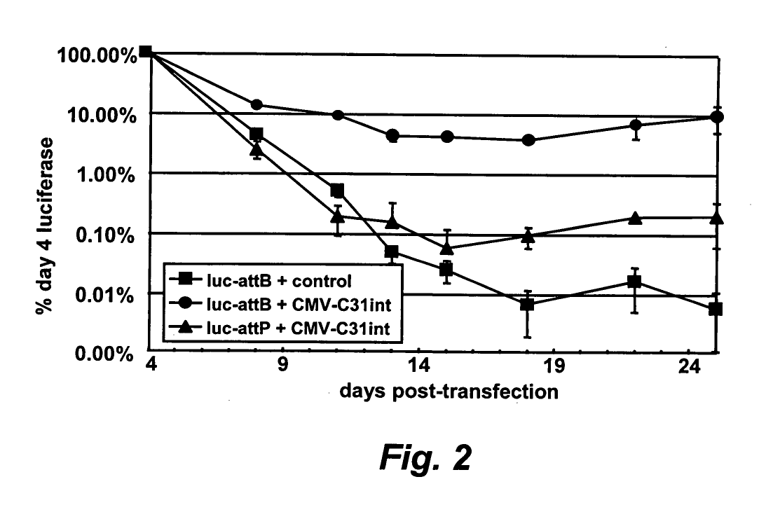

As can be seen in FIG. 2, in the absence of integrase, luciferase expression from a vector bearing attP or attB decreased to very low levels after several days. However, luciferase levels were persistent when the luciferase vector bearing attB was co-transfected with the integrase expression vector, indicating that the luciferase vector had stably integrated into the avian genome.

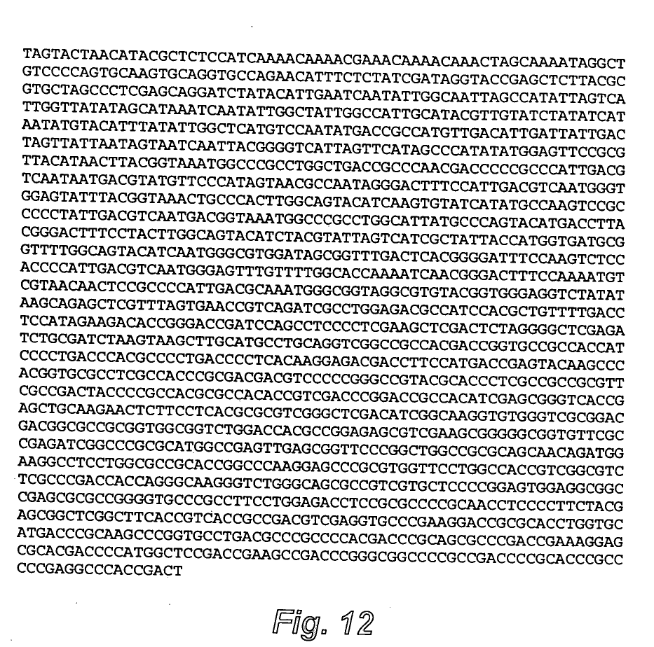

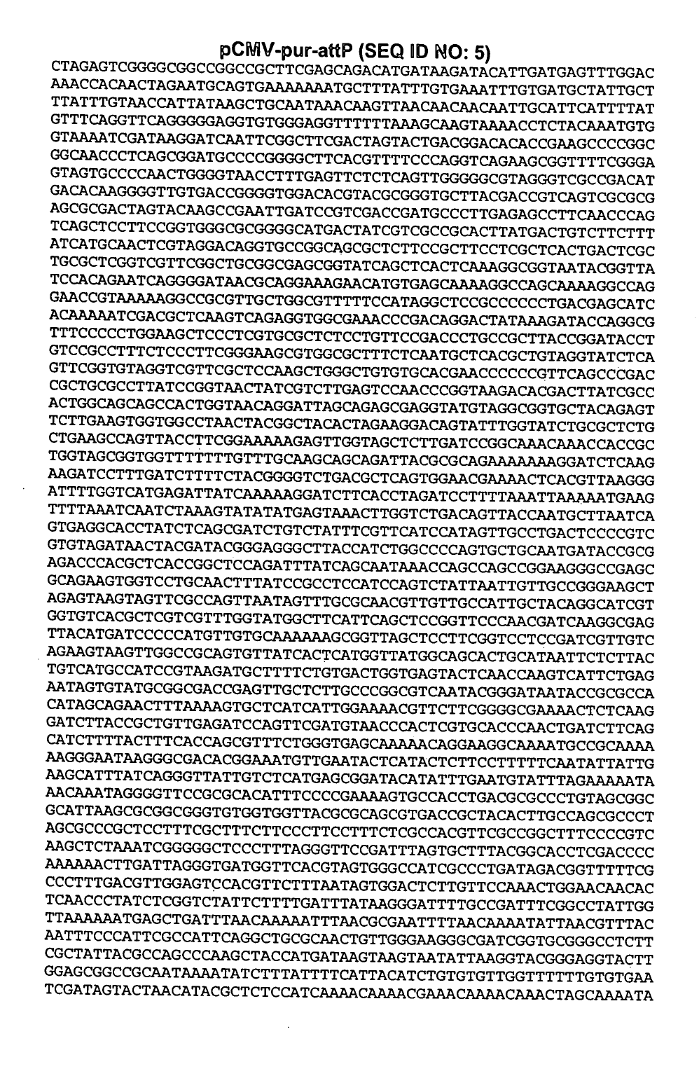

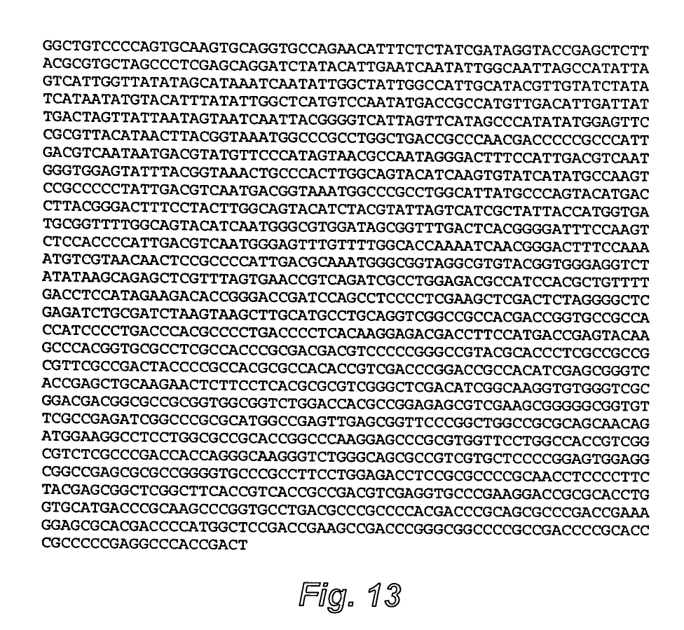

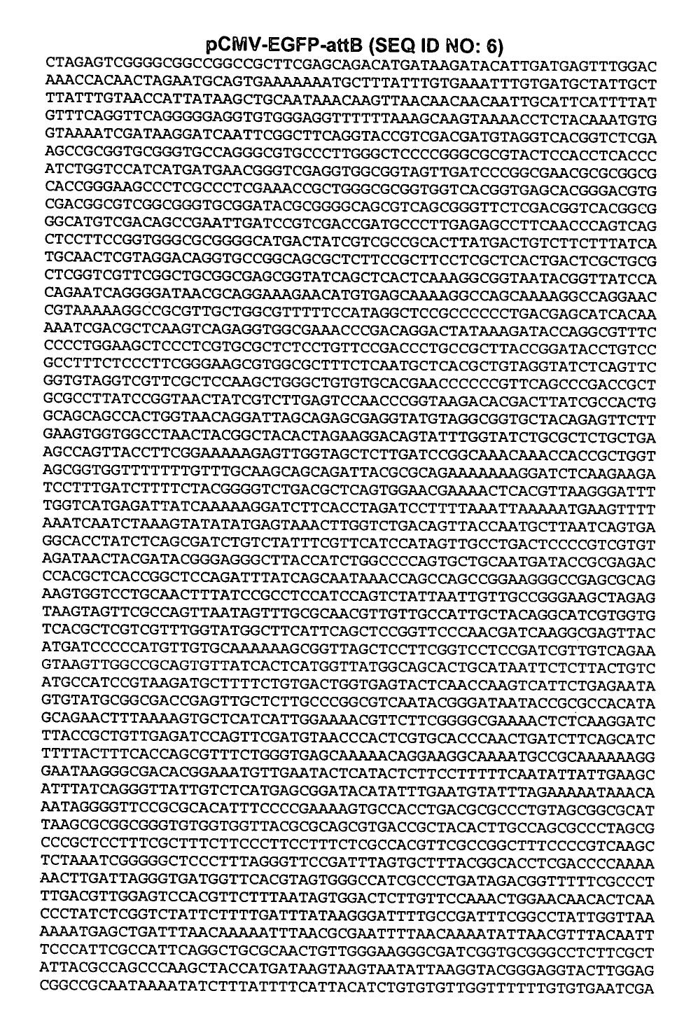

(b) A drug-resistance colony formation assay was used to quantitate integration efficiency. The puromycin resistance expression vector pCMV-pur was outfitted with an attB (SEQ ID NO: 4 shown in FIG. 12) or an attP (SEQ ID NO: 5 shown in FIG. 13) sites. Puromycin resistance vectors bearing attB sites were cotransfected with phiC31 integrase or a control vector into DF-1 cells. One day after transfection, puromycin was added. Puromycin resistant colonies were counted 12 days post-transfection.

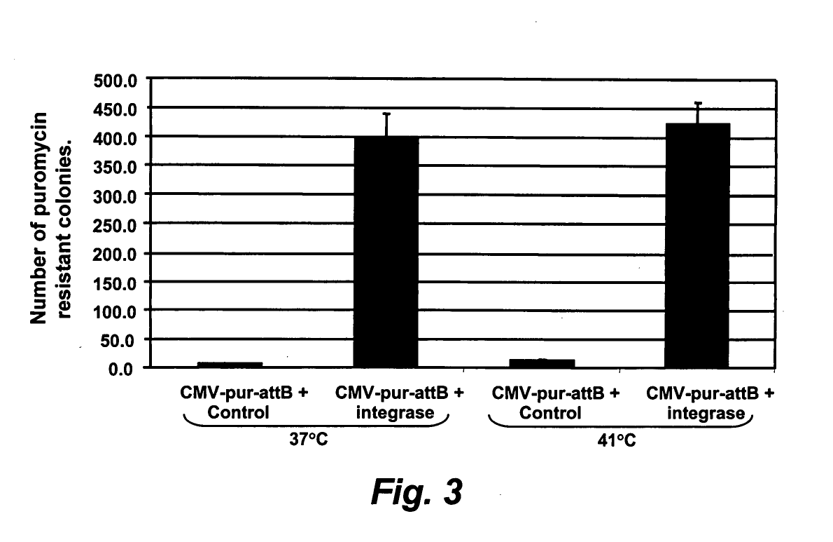

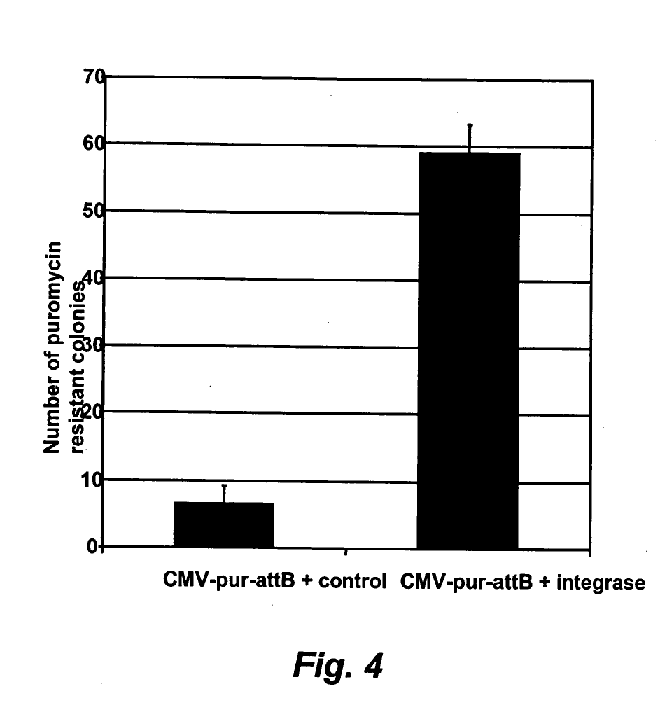

In the absence of co-transfected integrase expression, few DF-1 cell colonies were observed after survival selection. When integrase was co-expressed, multiple DF-1 cell colonies were observed, as shown in FIG. 3. Similar to the luciferase expression experiment, the attB sequence (but not the attP sequence) was able to facilitate integration of the plasmid into the genome. FIG. 3 also shows that phiC31 integrase functions at both 37° Celsius and 41° Celsius. Integrase also functions in quail cells using the puromycin resistance assay, as shown in FIG. 4.

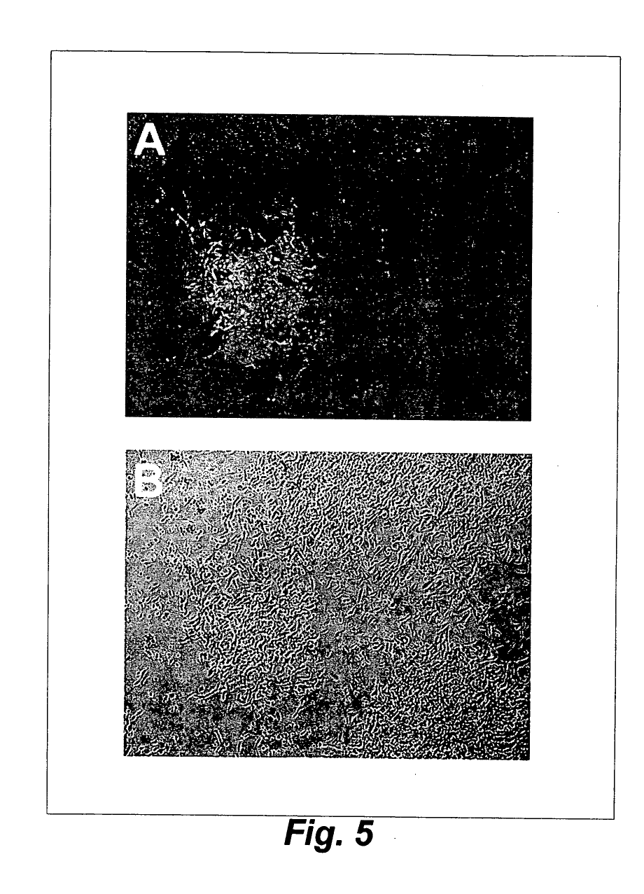

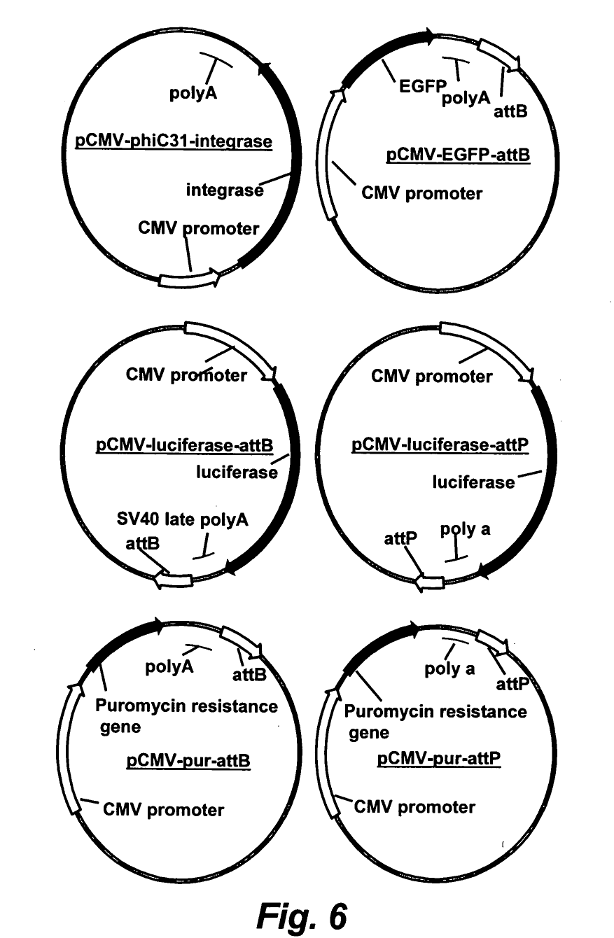

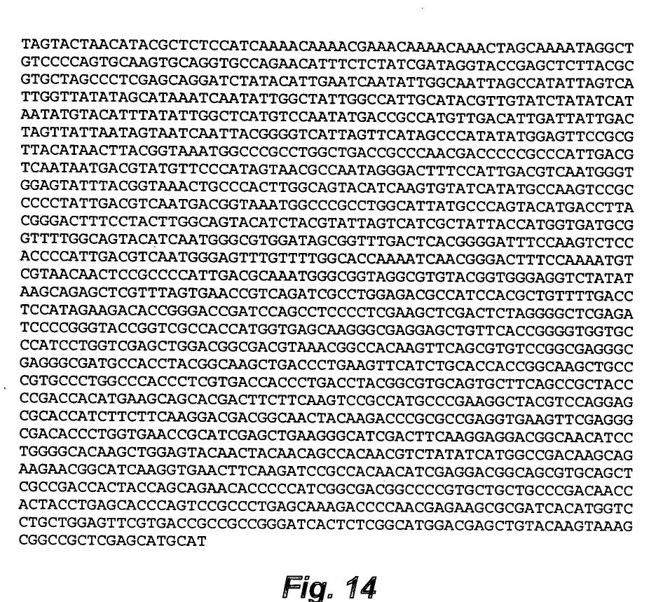

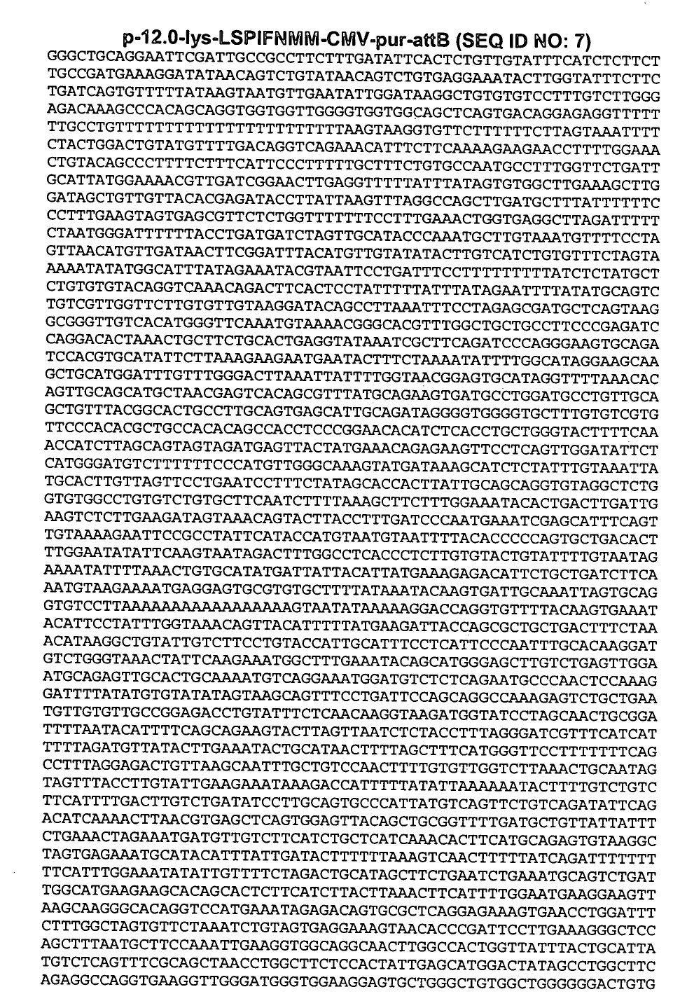

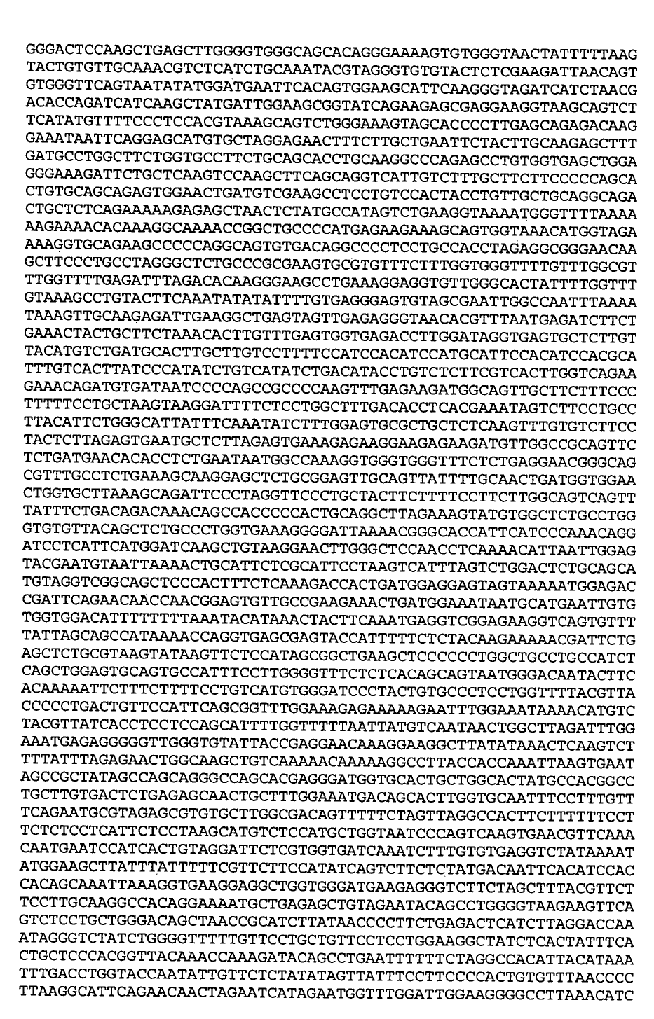

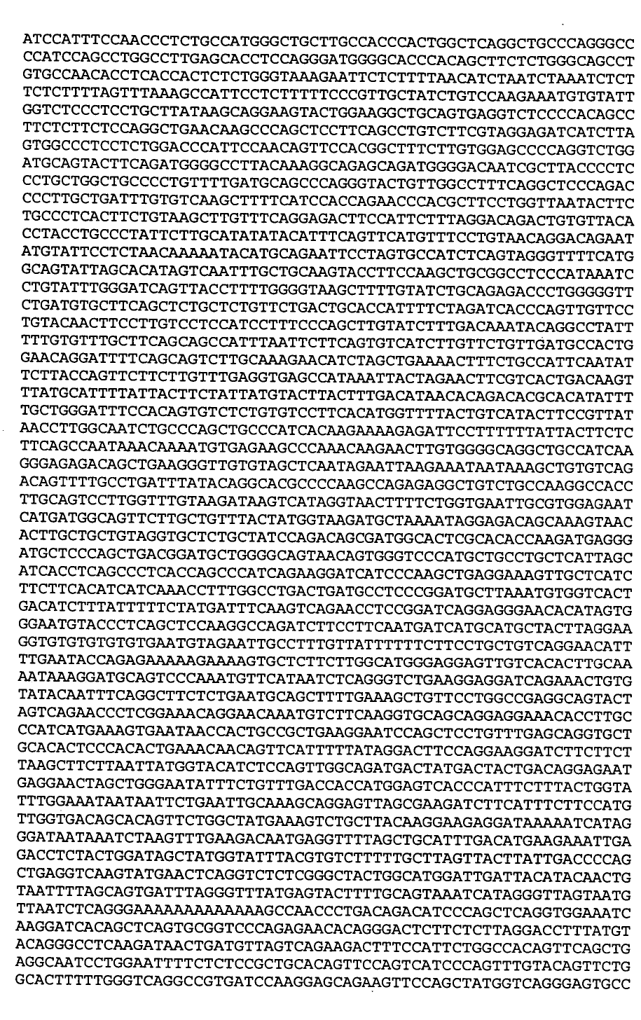

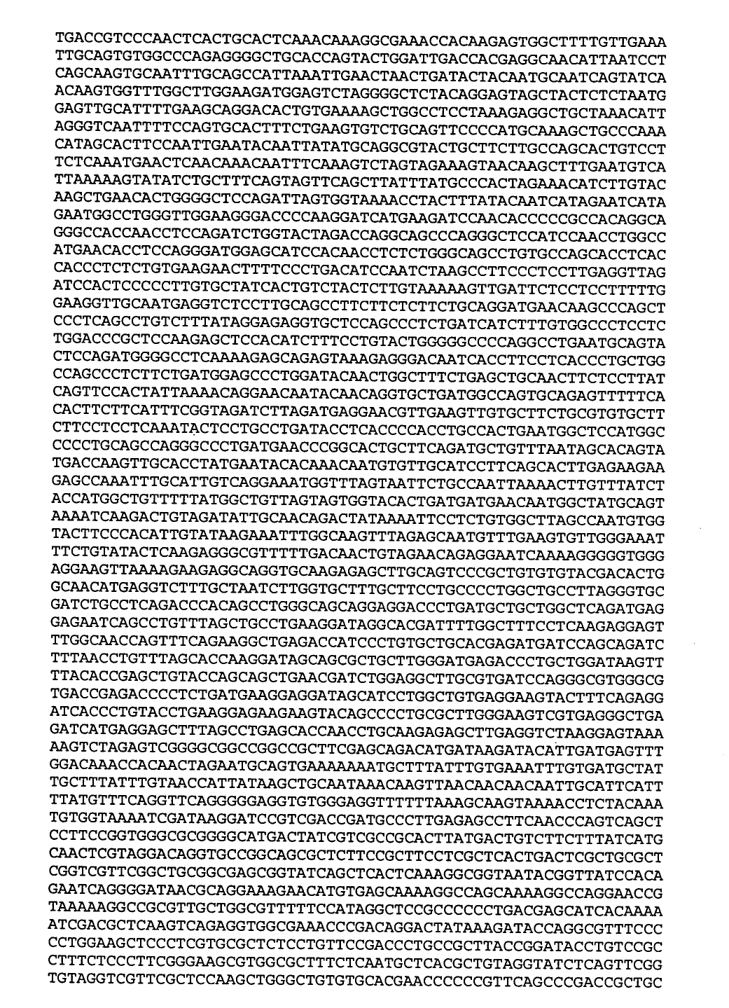

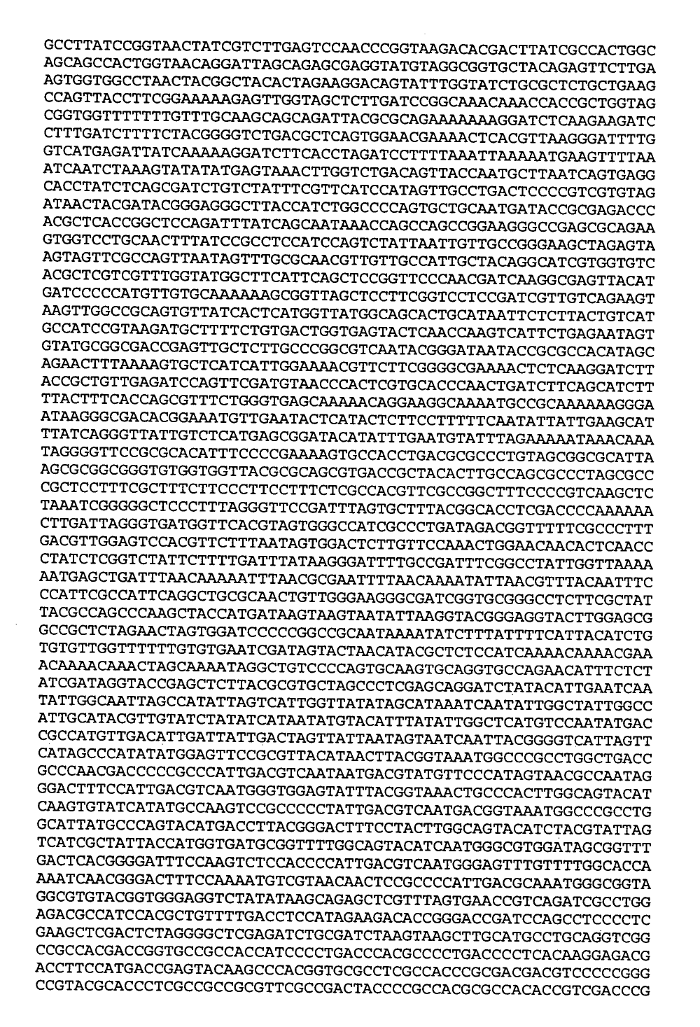

(c) The CMV-pur-attB vector (SEQ ID NO: 4) was also contransfected with an enhanced green fluorescent protein (EGFP) expression vector bearing an attB site (SEQ ID NO: 6 shown in FIG. 14) into DF-1 cells and the phiC31 integrase expression vector CMV-C31 int (SEQ ID NO: 1). After puromycin selection for 12 days, the colonies were viewed with UV light to determine the percentage of cells that expressed EGFP. Approximately 20% of puromycin resistant colonies expressed EGFP in all of the cells of the colony, as shown in FIG. 5, indicating that the integrase can mediate multiple integrations per cell.

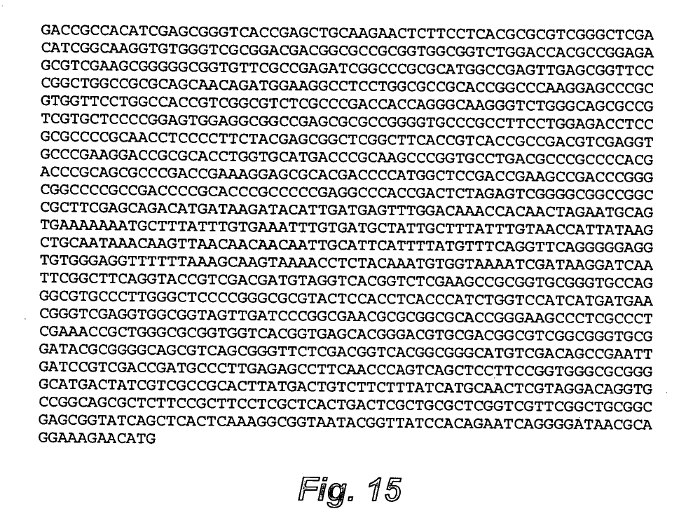

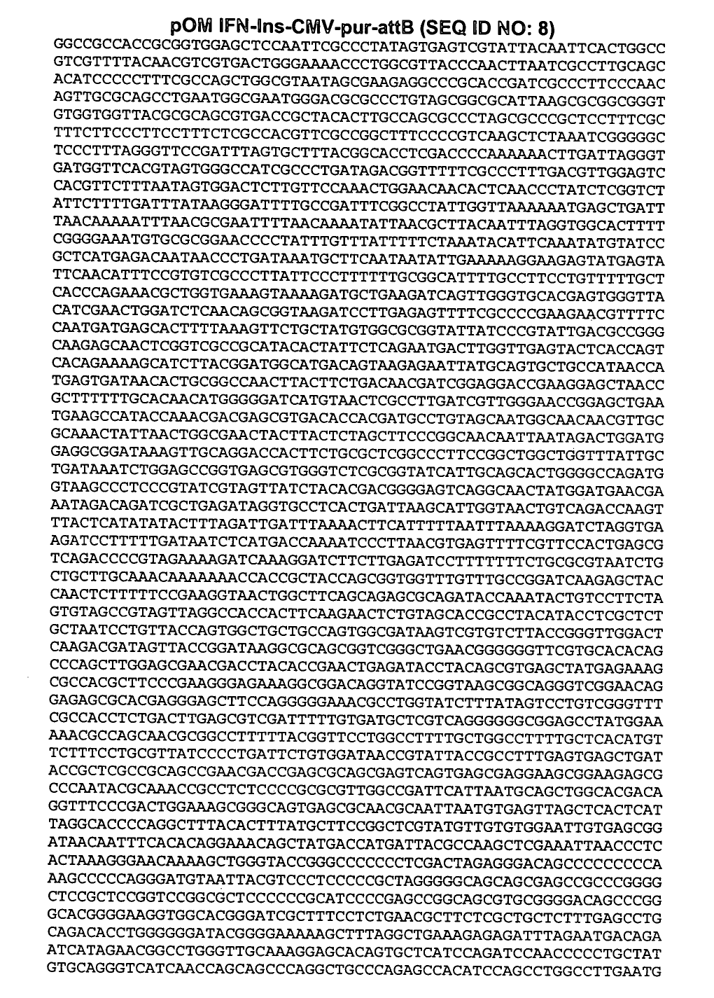

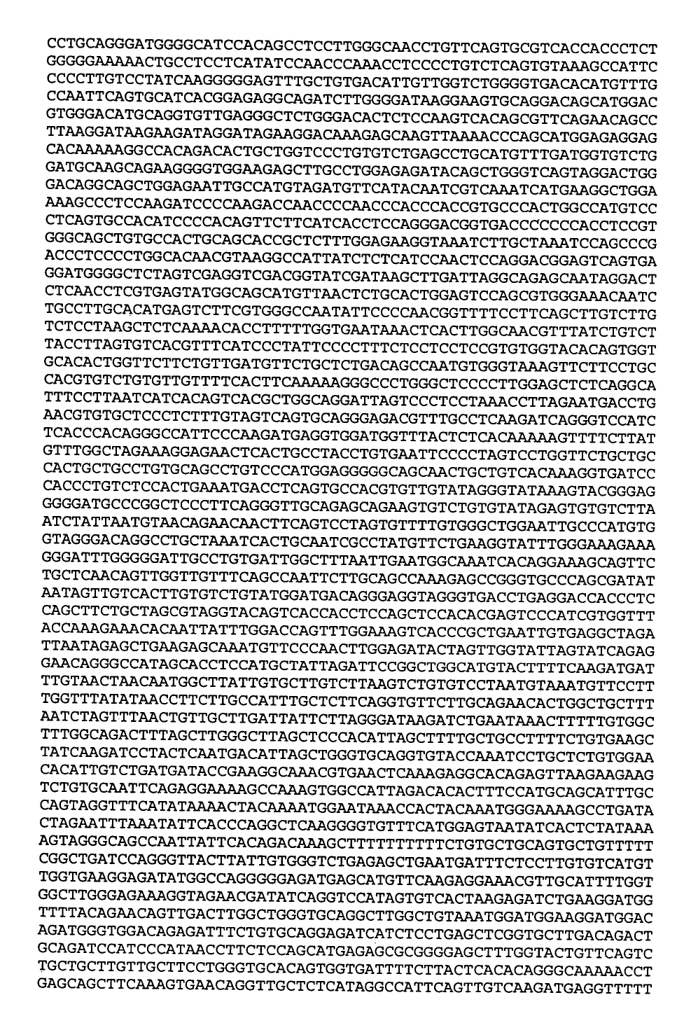

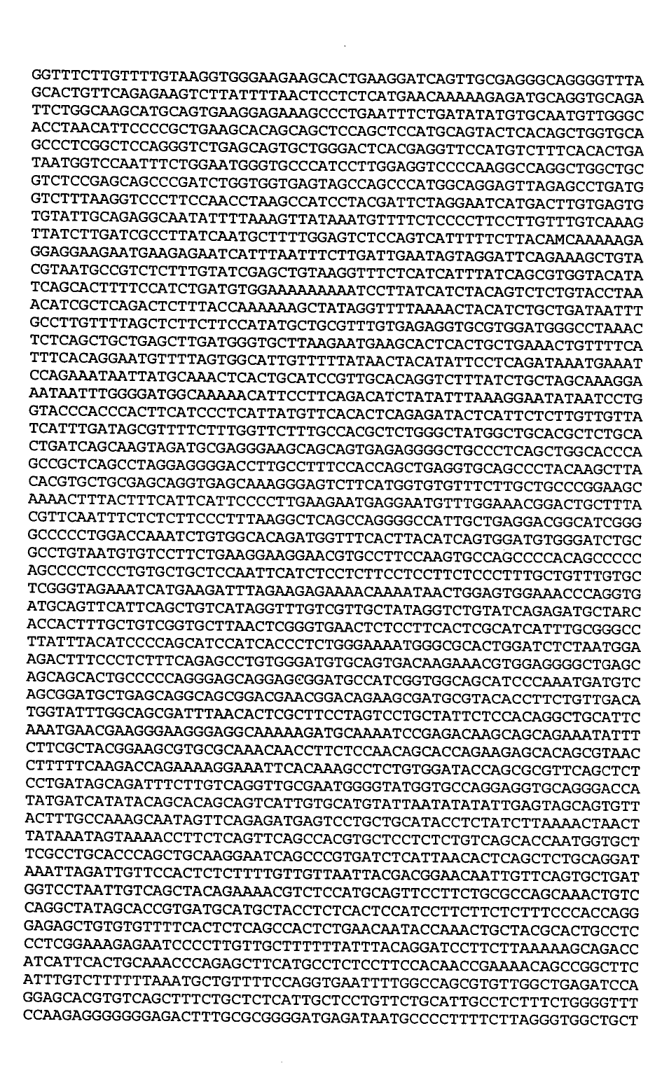

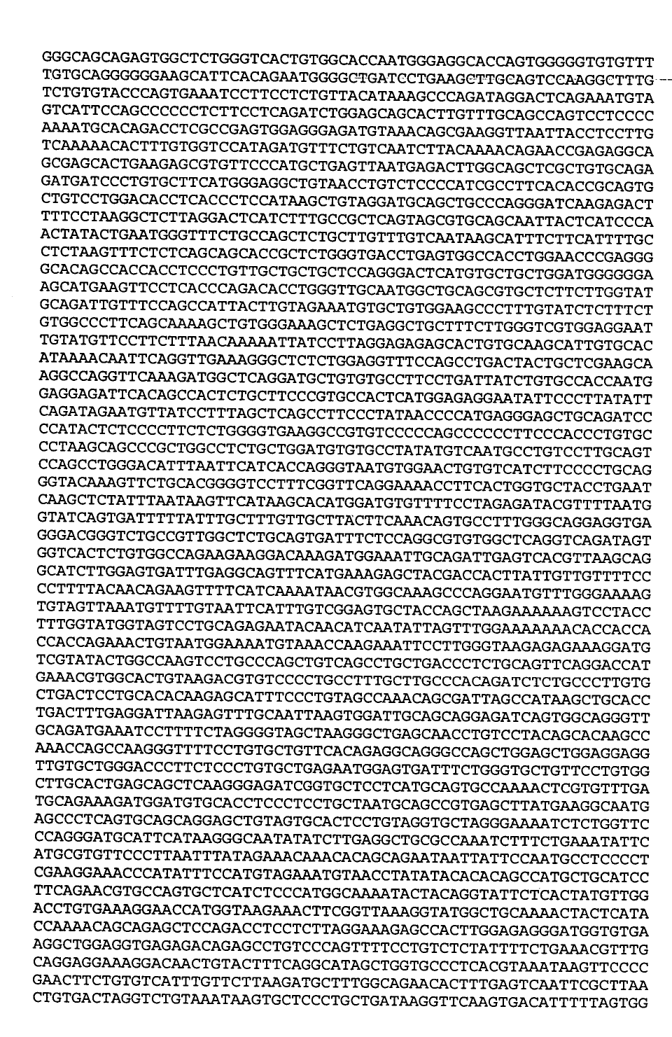

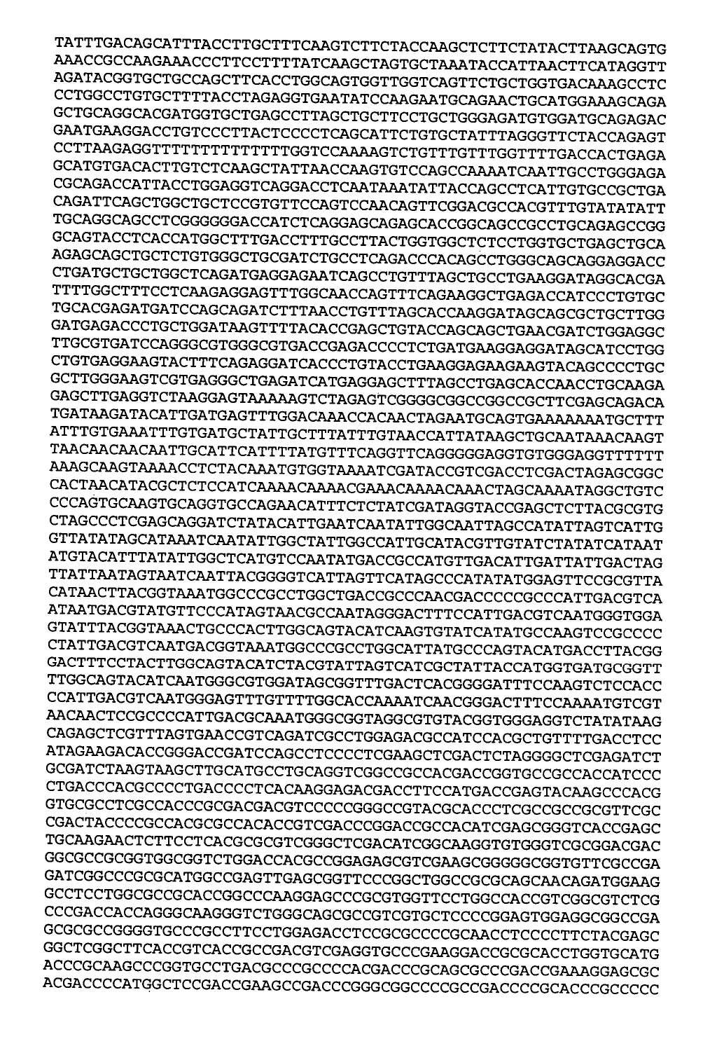

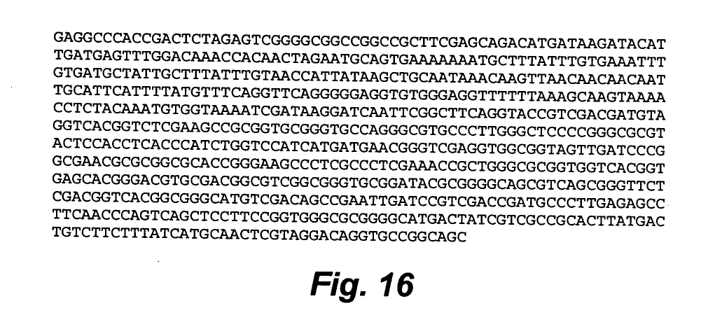

(d) PhiC31 integrase promoted the integration of large transgenes into avian cells. A puromycin expression cassette comprising a CMV promoter, puromycin resistance gene, polyadenylation sequence and the attB sequence was inserted into a vector containing a 12.0 kb lysozyme promoter and the human interferon a2b gene (SEQ ID NO: 7 shown in FIG. 15) and into a vector containing a 10.0 kb ovomucoid promoter and the human interferon α2b gene (SEQ ID NO: 8) as shown in FIG. 16.

DF-1 cells were transfected with donor plasmids of varying lengths bearing a puromycin resistance gene and an attB sequence in the absence or presence of an integrase expression plasmid. Puromycin was added to the culture media to kill those cells which did not contain a stably integrated copy of the puromycin resistance gene. Cells with an integrated gene formed colonies in the presence of puromycin in 7-12 days. The colonies were visualized by staining with methylene blue and the entire 60 mm culture dish was imaged.

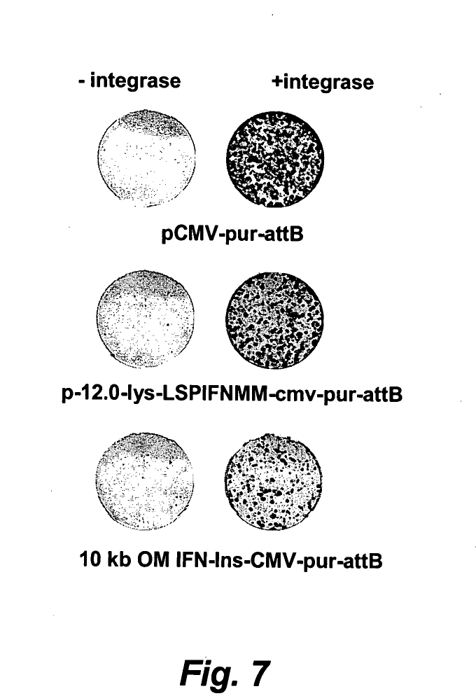

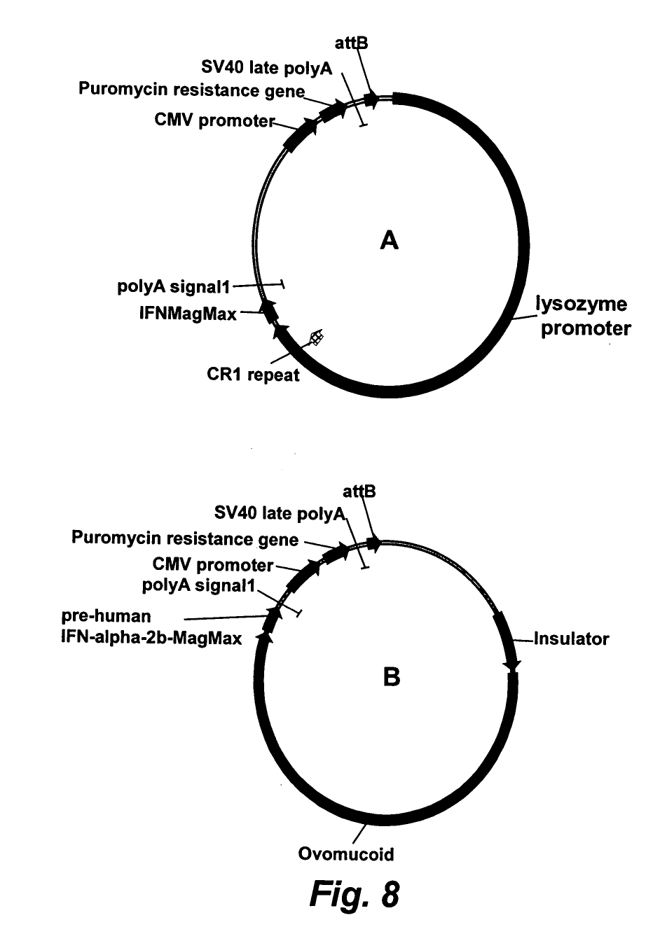

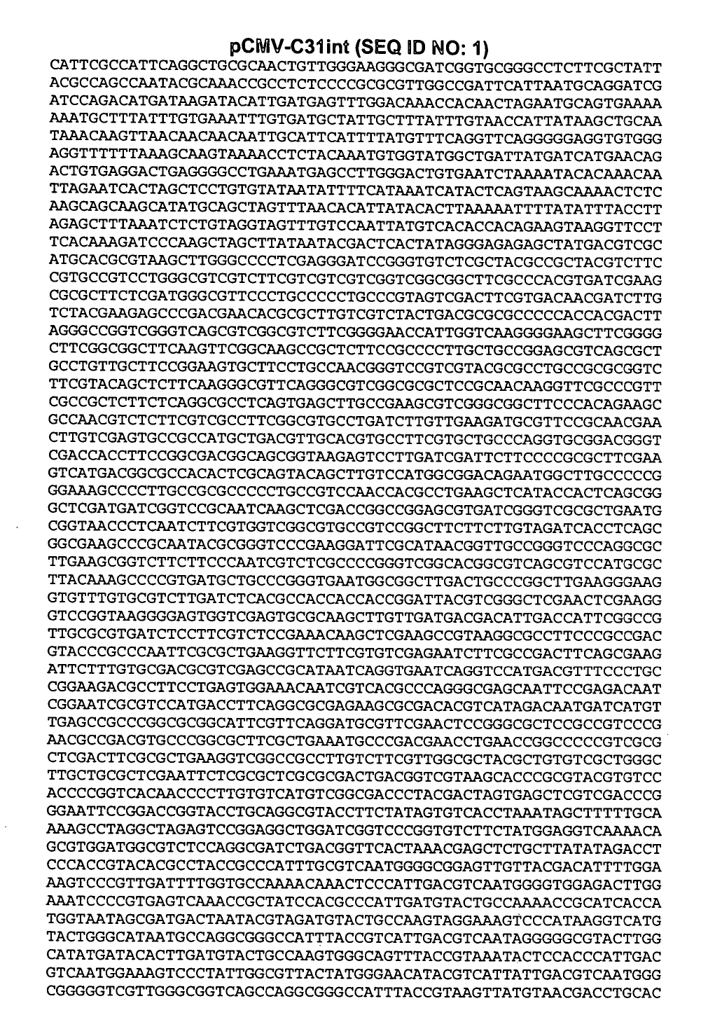

PhiC31 integrase mediated the efficient integration of both vectors as shown in FIG. 7.

### EXAMPLE 2

### Cell Culture Methods

DF-1 cells were cultured in DMEM with high glucose, 10% fetal bovine serum, 2 mM L-glutamine, 100 units/ml penicillin and 100 μg/ml streptomycin at 37° Celsius and 5% CO₂. A separate population of DF-1 cells was grown at 41° Celsius. These cells were adapted to the higher temperature for one week before they were used for experiments.

Quail QT6 cells were cultured in F10 medium (Gibco) with 5% newborn calf serum, 1% chicken serum heat inactivated (at 55° Celsius for 45 mins), 10 units/ml penicillin and 10 μg/ml streptomycin at 37° Celsius and 5% CO₂.

### EXAMPLE 3

### Selection and Assay Methods

(a) Puromycin selection assay: About 0.8×10⁶ DF-1 (chicken) or QT6 (quail) cells were plated in 60 mm dishes. The next day, the cells were transfected as follows:

10 to 50 ng of a donor plasmid and 1 to 10 μg of an Integrase-expressing plasmid DNA were mixed with 150 μl of OptiMEM. 15 μl of DMRIE-C was mixed with 150 μl of OptiMEM in a separate tube, and the mixtures combined and incubated for 15 mins. at room temperature.

While the liposome/DNA complexes were forming, the cells were washed with OptiMEM and 2.5 ml of OptiMEM was added. After 15 minutes, 300 μl of the DNA-lipid mixture was added drop wise to the 2.5 ml of OptiMEM covering the cell layers. The cells were incubated for 4-5 hours at either 37° Celsius or 41° Celsius, 5% CO₂. The transfection mix was replaced with 3 mls of culture media. The next day, puromycin was added to the media at a final concentration of 1 ug/ml, and the media replaced every 2 to 4 days. Puromycin resistant colonies were counted or imaged 10-12 days after the addition of puromycin.

(b) Luciferase assay: Chicken DF-1 or quail QT6 cells (0.8×10⁶) were plated in 60 mm dishes. Cells were transfected as described above. The cells from a plate were transferred to a new 100 mm plate when the plate became confluent, typically on day 3-4, and re-passaged every 3-4 days.

At each time point, one-third of the cells from a plate were replated, and one-third were harvested for the luciferase assay. The cells were pelleted in an eppendorf tube and frozen at −70° C.

The cell pellet was lysed in 200 μl of lysis buffer (25 mM Tris-acetate, pH7.8, 2 mM EDTA, 0.5% Triton X-100, 5% glycerol). Sample (5 μl) was assayed using the Promega BrightGlo reagent system.

(c) Visualization of EGFP: EGFP expression was visualized with an inverted microscope with FITC illumination [Olympus IX70, 100 W mercury lamp, HQ-FITC Band Pass Emission filter cube, exciter 480/40 nm, emission 535/50 nm, 20× phase contrast objective (total magnification was 2.5×10×20)].

(d) Staining of cell colonies: After colonies had formed, typically after 7-12 days of culture in puromycin medium, the cells were fixed in 2% formaldehyde, 0.2% glutaraldehyde for 15 mins, and stained in 0.2% methylene blue for 30 mins. followed by several washes with water. The plates were imaged using a standard CCD camera in visible light.

### EXAMPLE 4

### Production of Genetically Transformed Avian Cells

Avian stage X blastodermal cells are used as the cellular vector for the transgenes. Stage X embryos are collected and the cells dispersed and mixed with plasmid DNA. The transgenes are then introduced to blastodermal cells via electroporation. The cells are immediately injected back into recipient embryos.

The cells are not cultured for any time period to ensure that they remain capable of contributing to the germline of resulting chimeric embryos. However, because there is no culture step, cells that bear the transgene cannot be identified. Typically, only a small percentage of cells introduced to an embryo will bear a stably integrated transgene (0.01 to 1%). To increase the percentage of cells bearing a transgene, therefore, the transgene vector bears an attB site and is co-electroporated with a vector bearing the CMV promoter driving expression of the phiC31 transgene (CMV-C31int (SEQ ID NO: 1)). The integrase then drives integration of the transgene vector into the nuclear genome of the avian cell and increases the percentage of cells bearing a stable transgene.

(a) Preparation of Avian Stage X Blastodermal Cells:

i) Collect fertilized eggs from Barred Rock or White leghorn chickens (Gallus gallus) or quail (Japonica coturnix) within 48 hrs. of laying;

ii) Use 70% ethanol to clean the shells;

iii) Crack the shells and open the eggs;

iv) Remove egg whites by transferring yolks to opposite halves of shells, repeating to remove most of the egg whites;

v) Put egg yolks with embryo discs facing up into a 10 cm petri dish;

vi) Use an absorbent tissue to gently remove egg white from the embryo discs;

vii) Place a Whatman filter paper 1 ring over the embryos;

viii) Use scissors to cut the membranes along the outside edge of the paper ring while gently lifting the ring/embryos with a pair of tweezers;

ix) Insert the paper ring with the embryos at a 45 degrees angle into a petri dish containing PBS-G solution at room temperature;

x) After ten embryo discs are collected, gently wash the yolks from the blastoderm discs using a Pasteur pipette under a stereo microscope;

xi) Cut the discs by a hair ring cutter (a short piece of human hair is bent into a small loop and fastened to the narrow end of a Pasteur pipette with Parafilm);

xii) Transfer the discs to a 15 ml sterile centrifuge tube on ice;

xiii) Place 10 to 15 embryos per tube and allow to settle to the bottom (about 5 mins.);

xiv) Aspirate the supernatant from the tube;

xv) Add 5 mls of ice-cold PBS without Ca⁺⁺ and Mg⁺⁺, and gently pipette 4 to 5 times using a 5 mls pipette;

xvi) Incubate in ice for 5-7 mins. to allow the blastoderms to settle, and aspirate the supernatant;

xvii) Add 3 mls of ice cold 0.05% trypsin/0.02% ETDA to each tube and gently pipette 3 to 5 times using a 5 ml pipette;

xviii) Put the tube in ice for 5 mins. and then flick the tube by finger 40 times. Repeat;

xix) Add 0.5 mls FBS and 3-5 mls BDC medium to each tube and gently pipette 5-7 times using a 5 ml pipette;

xx) Spin at 500 rpm (RCF 57×g) at 4° Celsius for 5 mins;

xxi) Remove the supernatant and add 2 mls ice cold BDC medium into each tube; and

xxii) Resuspend the cells by gently pipetting 20-25 times; and

xxiii) Determine the cell titer by hemacytometer and ensure that about 95% of all BDCs are single cells, and not clumped.

(b) Transfection of Linearized Plasmids into Blastodermal Cells by Small Scale Electroporation:

i) Centrifuge the blastodermal cell suspension from step (xxiii) above at RCF 57×g, 4° Celsius, for 5 mins;

ii) Resuspend cells to a density of 1-3×10⁶ per ml with PBS without Ca²⁺ and Mg²⁺;

iii) Add linearized DNA, 1-30 μg per 1-3×10⁵ blastodermal cells in an eppendorf tube at room temperature. Add equimolar molar amounts of the non-linearized transgene plasmid bearing an attB site, and an integrase expression plasmid;

iv) Incubate at room temperature for 10 mins;

v) Aliquot 100 μl of the DNA-cell mixture to a 0.1 cm cuvette at room temperature;

vi) Electroporate at 240 V and 25 μFD (or 100 V and 125 μFD for quail cells) using, for example, a Gene Pulser II™ (BIO-RAD).

vii) Incubate the cuvette at room temperature for 1-10 mins.

viii) Before the electroporated cells are injected into a recipient embryo, they are transferred to a eppendorf tube at room temperature. The cuvette is washed with 350 μl of media, which is transferred to the eppendorf, spun at room temperature and re-suspended in 0.01-0.3 ml medium;

ix) Inject 1-10 μl of cell suspension into the subgerminal cavity of an non-irradiated or, preferably, an irradiated (e.g., with 300-900 rads) stage X egg. Shell and shell membrane are removed and, after injection, resealed according to U.S. Pat. No. 6,397,777 incorporated herein by reference in its entirety; and

x) The egg is then incubated to hatching.

(c) Blastodermal Cell Culture Medium:

i) 409.5 mls DMEM with high glucose, L-glutamine, sodium pyruvate, pyridoxine hydrochloride;

ii) 5 mls Men non-essential amino acids solution, 10 mM;

iii) 5 mls Penicillin-streptomycin 5000 U/ml each;

iv) 5 mls L-glutamine, 200 mM;

v) 75 mls fetal bovine serum; and

vi) 0.5 mls β-mercaptoethanol, 11.2 mM.

### EXAMPLE 5

### Transfection of Stage X Embryos with attB Plasmids

(a) DNA-PEI: Twenty-five μg of a phage phiC31 integrase expression plasmid (pCMV-int), and 25 μg of a luciferase-expressing plasmid (pβ-actin-GFP-attB) are combined in 200 μl of 28 mM Hepes (pH 7.4). The DNA/Hepes is mixed with an equal volume of PEI which has been diluted 10-fold with water. The DNA/Hepes/PEI is incubated at room temperature for 15 mins Three to seven μl of the complex are injected into the subgerminal cavity of windowed stage X white leghorn eggs which are then sealed and incubated as described in U.S. Pat. No. 6,397,777. The complexes will also be incubated with blastodermal cells isolated from stage X embryos which are subsequently injected into the subgerminal cavity of windowed irradiated stage X white leghorn eggs. Injected eggs are sealed and incubated as described above.

(b) Adenovirus-PEI:

Two μg of a phage phiC31 integrase expression plasmid (pCMV-int), 2 μg of a GFP expressing plasmid (pβ-actin-GFP-attB) and 2 μg of a luciferase expressing plasmid (pGLB) were incubated with 1.2 μl of JetPEI™ in 50 μl of 20 mM Hepes buffer (pH7.4). After 10 mins at 25° C., 3×10⁹ adenovirus particles (Ad5-Null, Qbiogene) were added and the incubation continued for an additional 10 mins. Embryos are transfected in ovo or ex ovo as described above.

### EXAMPLE 6

### Stage I Cytoplasmic Injection

Production of transgenic chickens by cytoplasmic DNA injection using DNA injection directly into the germinal disk as described in Sang et al., Mol. Reprod. Dev., 1: 98-106 (1989); Love et al., Biotechnology, 12: 60-63 (1994) incorporated herein by reference in their entireties.

In the method of the present invention, fertilized ova, and preferably stage I embryos, are isolated from euthanized hens 45 mins. to 4 hrs. after oviposition of the previous egg. Alternatively, eggs were isolated from hens whose oviducts have been fistulated according to the techniques of Gilbert & Wood-Gush, J. Reprod. Fertil., 5: 451-453 (1963) and Pancer et al., Br. Poult. Sci., 30: 953-7 (1989) incorporated herein in their entireties.

An isolated ovum was placed in dish with the germinal disk upwards. Ringer's buffer medium was then added to prevent drying of the ovum. Any suitable microinjection assembly and methods for microinjecting and reimplanting avian eggs are useful in the method of cytoplasmic injection of the present invention. A particularly suitable apparatus and method for use in the present invention is described in U.S. patent application Ser. No: 09/919,143 (“the '143 Application) and incorporated herein by reference in its entirety. The avian microinjection system described in the '143 Application allowed the loading of a DNA solution into a micropipette, followed by prompt positioning of the germinal disk under the microscope and guided injection of the DNA solution into the germinal disk. Injected embryos could then be surgically transferred to a recipient hen as described, for example, in Olsen & Neher, J. Exp. Zool., 109: 355-66 (1948) and Tanaka et al., J. Reprod. Fertil., 100: 447-449 (1994). The embryo was allowed to proceed through the natural in vivo cycle of albumin deposition and hard-shell formation. The transgenic embryo is then laid as a hard-shell egg which was incubated until hatching of the chick. Preferably, injected embryos were surgically transferred to recipient hens via the ovum transfer method of Christmann et al. in PCT/US01/26723, the contents of which are incorporated by reference in its entirety, and hard shell eggs were incubated and hatched.

Approximately 25 nl of DNA solution (about 60 ng/μl) with either integrase mRNA or protein were injected into a germinal disc of stage I White Leghorn embryos obtained 90 minutes after oviposition of the preceding egg. Typically the concentration of integrase mRNA used was 100 ng/μl, and the concentration of integrase protein was 66 ng/μl.

To synthesize the integrase mRNA, a plasmid template encoding the integrase protein was linearized at the 3′ end of the transcription unit. mRNA was synthesized, capped and a polyadenine tract added using the mMESSAGE mMACHINE T7 Ultra Kit™ (Ambion, Austin, Tex.). The mRNA was purified by extraction with phenol and chloroform and precipitiated with isopropanol. The integrase protein was expressed in E. coli and purified as described by Thorpe et al., Mol. Microbiol., 38: 232-241 (2000).

A plasmid encoding for the integrase protein is transfected into the target cells. However, since the early avian embryo transcriptionally silent until it reaches about 22,000 cells, injection of the integrase mRNA or protein was expected to result in better rates of transgenesis, as shown in the Table 2 below.

The chicks produced by this procedure were screened for the presence of the injected transgene using a high throughput PCR-based screening procedure as described in Harvey et al., Nature Biotech., 20: 396-399 (2002).

TABLE 2
Summary of cytoplasmic injection results using different integrase
strategies

| Experimental | Ovum | Hard shells | Chicks | Transgenic |
|---|---|---|---|---|
| group | transfers | produced (%) | hatched (%)* | chicks (%)^{‡} |
| No Integrase | 5164 | 3634 (70%) | 500 (14%) | 58 (11.6%) |
| Integrase | 1109 | 833 (75%) | 115 (13.8%) | 19 (16.5%) |
| mRNA | | | | |
| Integrase | 374 | 264 (70.6%) | 47 (17.8%) | 16 (34%) |
| protein | | | | |

*Percentages based on the number of hard shells
^{‡}Percentages based on the number of hatched birds

### EXAMPLE 7

### Characterization of phiC31 Integrase-Mediated Integration Sites in the Chicken Genome

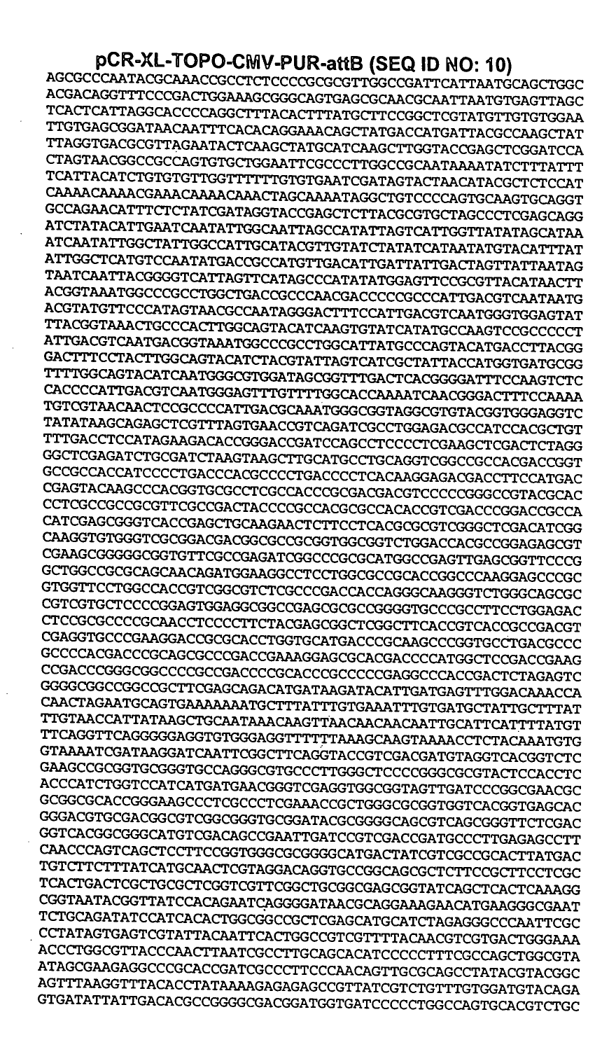

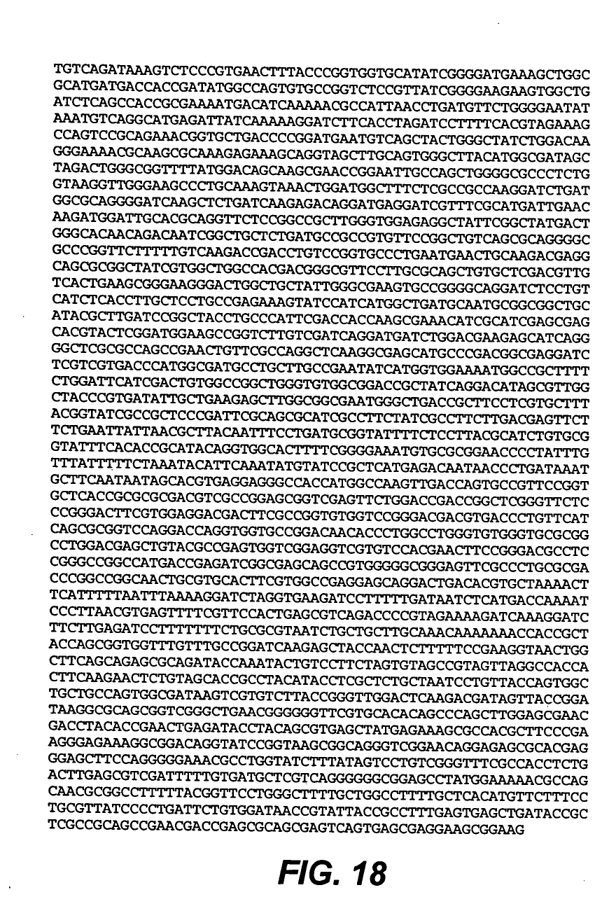

To characterize phiC31-mediated integration into the chicken genome, a plasmid rescue method was used to isolate integrated plasmids from transfected and selected chicken fibroblasts. Plasmid pCR-XL-TOPO-CMV-pur-attB (SEQ ID NO: 10, shown in FIG. 18) does not have BamHI or BglII restriction sites. Genomic DNA from cells transformed with pCR-XL-TOPO-CMV-pur-attB was cut with BamHI or BglII (either or both of which would cut in the flanking genomic regions) and religated so that the genomic DNA surrounding the integrated plasmid would be captured into the circularized plasmid. The flanking DNA of a number of plasmids were then sequenced.

DF-1 cells (chicken fibroblasts), 4×10⁵ were transfected with 50 ng of pCR-XL-TOPO-CMV-pur-attB and 1 μg of pCMV-int. The following day, the culture medium was replaced with fresh media supplemented with 1 μg/ml puromycin. After 10 days of selection, several hundred puromycin-resistant colonies were evident. These were harvested by trypsinzation, pooled, replated on 10 cm plates and grown to confluence. DNA was then extracted.

Isolated DNA was digested with BamHI and BglII for 2-3 hrs, extracted with phenol:chloroform:isoamyl alcohol chloroform:isoamyl alcohol and ethanol precipitated. T4 DNA ligase was added and the reaction incubated for 1 hr at room temperature, extracted with phenol:chloroform:isoamyl alcohol and chloroform:isoamyl alcohol, and precipitated with ethanol. 5 μl of the DNA suspended in 10 μl of water was electroporated into 25 μl of Genehogs™ (Invitrogen) in an 0.1 cm cuvette using a GenePulser II (Biorad) set at 1.6 kV, 100 ohms, 25 uF and plated on Luria Broth (LB) plates with 5 μg/ml phleomycin (or 25 μg/ml zeocin) and 20 μg/ml kanamycin. Approximately 100 individual colonies were cultured, the plasmids extracted by standard miniprep techniques and digested with Xba I to identify clones with unique restriction fragments.

Thirty two plasmids were sequenced with the primer attB-for (5′-TACCGTCGACGATGTAGGTCACGGTC-3′) (SEQ ID NO: 12) which allows sequencing across the crossover site of attB and into the flanking genomic sequence. All of plasmids sequenced had novel sequences inserted into the crossover site of attB, indicating that the clones were derived from plasmid that had integrated into the chicken genome via phiC31 integrase-mediated recombination.

The sequences were compared with sequences at GenBank using Basic Local Alignment Search Tool (BLAST). Most of the clones harbored sequences homologous to Gallus genomic sequences in the TRACE database.

### EXAMPLE 8

### Insertion of a Wild-Type attP Site into the Avian Genome Augments Integrase-Mediated Integration and Transgenesis

The chicken B-cell line DT40 cells (Buerstedde et al., E.M.B.O. J, 9: 921-927 (1990)) are useful for studying DNA integration and recombination processes (Buerstedde & Takeda, Cell, 67:179-88 (1991)). DT40 cells were engineered to harbor a wild-type attP site isolated from the Streptomyces phage phiC31. Two independent cell lines were created by transfection of a linearized plasmid bearing an attP site linked to a CMV promoter driving the resistance gene to G418 (DT40-NLB-attP) or bearing an attP site linked to a CMV promoter driving the resistance gene for puromycin (DT40-pur-attP). The transfected cells were cultured in the presence of G418 or puromycin to enrich for cells bearing an attP sequence stably integrated into the genome.

A super-coiled luciferase vector bearing an attB (SEQ ID NO: 2 shown in FIG. 10) was co-transfected, together with an integrase expression vector CMV-C31int (SEQ ID NO: 1) or a control, non-integrase expressing vector (CMV-BL) into wild-type DT40 cells and the stably transformed lines DT40-NLB-attP and DT40-pur-attP.

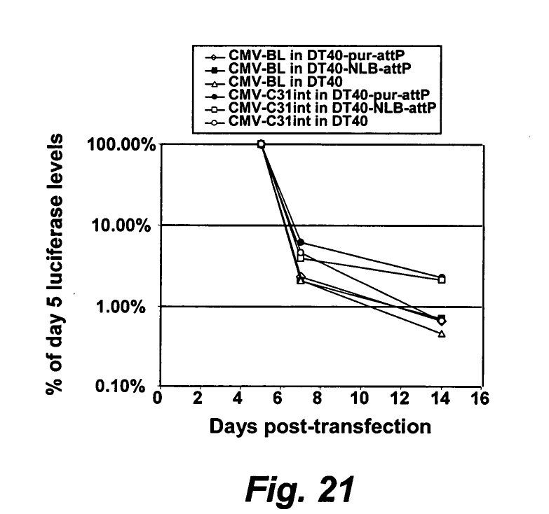

Cells were passaged at 5, 7 and 14 days post-transfection and about one third of the cells were harvested and assayed for luciferase. The expression of luciferase was plotted as a percentage of the expression measured 5 days after transfection. As can be seen in FIG. 21, in the absence of integrase, or in the presence of integrase but in the DT40 cells lacking an inserted wild-type attP site, luciferase expression from a vector bearing attB progressively decreased to very low levels. However, luciferase levels were persistent when the luciferase vector bearing attB was co-transfected with the integrase expression vector into the attP bearing cell lines DT40-NLB-attP and DT40-pur-attP. Inclusion of an attP sequence in the avian genome augments the level of integration efficiency beyond that afforded by the utilization of endogenous pseudo-attP sites.

### EXAMPLE 9

### Generation of attP Transgenic Cell Line and Birds Using an NLB Vector

The NLB-attP retroviral vector can be injected into stage X chicken embryos laid by pathogen-free hens. A small hole is drilled into the egg shell of a freshly laid egg, the shell membrane cut away and the embryo visualized by eye. With a drawn needle attached to a syringe, 1 to 10 μl of concentrated retrovirus, approximately 2.5×10⁵ IU, is injected into the subgerminal cavity of the embryo. The egg shell is resealed with a hot glue gun. Suitable methods for the manipulation of avian eggs, including opening and resealing hard shell eggs are described in U.S. Pat. Ser. Nos: 5,897,998 and 6,397,777 which are herein incorporated by reference in their entireties.

Typically, 25% of embryos hatch 21 days later. The chicks are raised to sexual maturity and semen samples are taken. Birds that have a significant level of the transgene in sperm DNA will be identified, typically by a PCR-based assay. Ten to 25% of the hatched roosters will be able to give rise to G1 transgenic offspring, 1 to 20% of which may be transgenic. DNA extracted from the blood of G1 offspring is analyzed by PCR and Southern analysis to confirm the presence of the intact transgene. Several lines of transgenic roosters, each with a unique site of attP integration, are then bred to non-transgenic hens, giving 50% of G2 transgenic offspring. Transgenic G2 hens and roosters from the same line can be bred to produce G3 offspring homozygous for the transgene. Homozygous offspring will be distinguished from hemizygous offspring by quantitative PCR. The same procedure can be used to integrate an attB or attP site into transgenic birds.

### EXAMPLE 10

### Expression of Immunoglobulin Chain Polypeptides by Transgenic Chickens

Bacterial artificial chromosomes (BACs) containing a 70 kbp segment of the chicken ovomucoid gene with the light and heavy chain cDNAs for a human monoclonal antibody inserted along with an internal ribosome entry site into the 3′ untranslated region of the ovomucoid gene were equipped with the attB sequence. The heavy and light chain cDNAs were inserted into separate ovomucoid BACs such that expression of an intact monoclonal antibody requires the presence of both BACs in the nucleus.

Several hens produced by coinjection of the attB-bearing ovomucoid BACs and integrase-encoding mRNA into stage I embryos produced intact monoclonal antibodies in their egg white. One hen, which had a high level of the light chain ovomucoid BAC in her blood DNA as determined by quantitative PCR particularly expressed the light chain portion of the monoclonal antibody in the egg white at a concentration of 350 nanograms per ml, or approximately 12 μg per egg.

### EXAMPLE 11

### Stage I Cytoplasmic Injection with Integrase Activity and PEI

Production of transgenic chickens by cytoplasmic DNA injection directly into the germinal disk was done as described in Example 6.

Approximately 25 nl of aqueous DNA (about 60ng/μl) which includes a transgene is placed in solution with integrase mRNA or integrase protein was mixed with an equal volume of PEI that had been diluted ten fold. The mixture was injected into a germinal disc of stage I White Leghorn embryos obtained about 90 minutes after oviposition of the preceding egg. Typically the concentration of integrase mRNA used was about 100 ng/μl, and the concentration of integrase protein was about 66 ng/μl. The integrase mRNA was synthesized according to Example 6.

Transgenic chicks produced by this procedure using: integrase mRNA/PEI and integrase protein/PEI showed positive results for the presence of heterologously expressed protein in the blood, semen and egg white.

### EXAMPLE 12

### Stage I Cytoplasmic Injection with Integrase Activity and NLS

Production of transgenic chickens by cytoplasmic DNA injection directly into the germinal disk was done as described in Example 6.

DNA which includes a transgene was suspended in 0.25 M KCl and SV40 T antigen nuclear localization signal peptide (NLS peptide, amino acid sequence CGGPKKKRKVG (SEQ ID NO: 13)) was added to achieve a peptide DNA molar ratio of 100:1. The DNA (about 60 ng/μl) was allowed to associate with the SV40 T antigen NLS peptide by incubating at 25 degrees C. for about 15 minutes.

Integrase mRNA or integrase protein was added to approximately 25 nl of an aqueous DNA/NLS solution, typically, to produce a final concentration of integrase mRNA of about 50 ng/μl, or an integrase protein concentration of about 33 ng/μl. The mixture was injected into a germinal disc of stage I White Leghorn embryos obtained about 90 minutes after oviposition of the preceding egg. The integrase mRNA was synthesized as according to Example 6.

Transgenic chicks produced by this procedure using: integrase mRNA/NLS and integrase protein/NLS showed positive results for the presence of heterologously expressed protein in blood, semen and egg white.

### EXAMPLE 13

### Dispersing of Plasmid DNA in Avian Stage I Embryos

DNA samples are Cy3 labeled with a Cy3 ULS labeling kit (Amersham Pharmacia Biotech). Briefly, plasmid DNA (1 μg) is first sheared to approximately 100 to 500 bp fragments by sonication. Resulting DNA is incubated at 65° C. for 15 min in Cy3 ULS labeling solution and unincorporated Cy3 dye is removed by spin column chromatography (CentriSep, Princeton Separations). The distribution of the DNA in stage I avian embryos was visualized after introduction into the stage I avian embryo. Enough high molecular weight or low molecular weight PEI was added to the DNA to coat the DNA. Typically, PEI was added to the DNA to a concentration of about 5%.

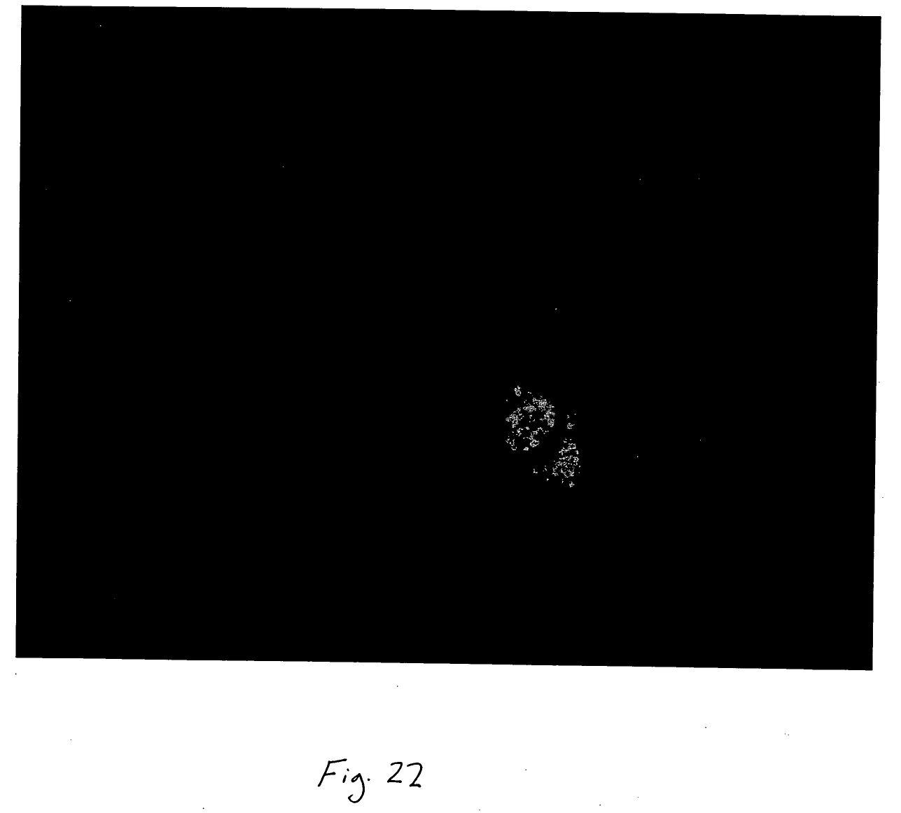

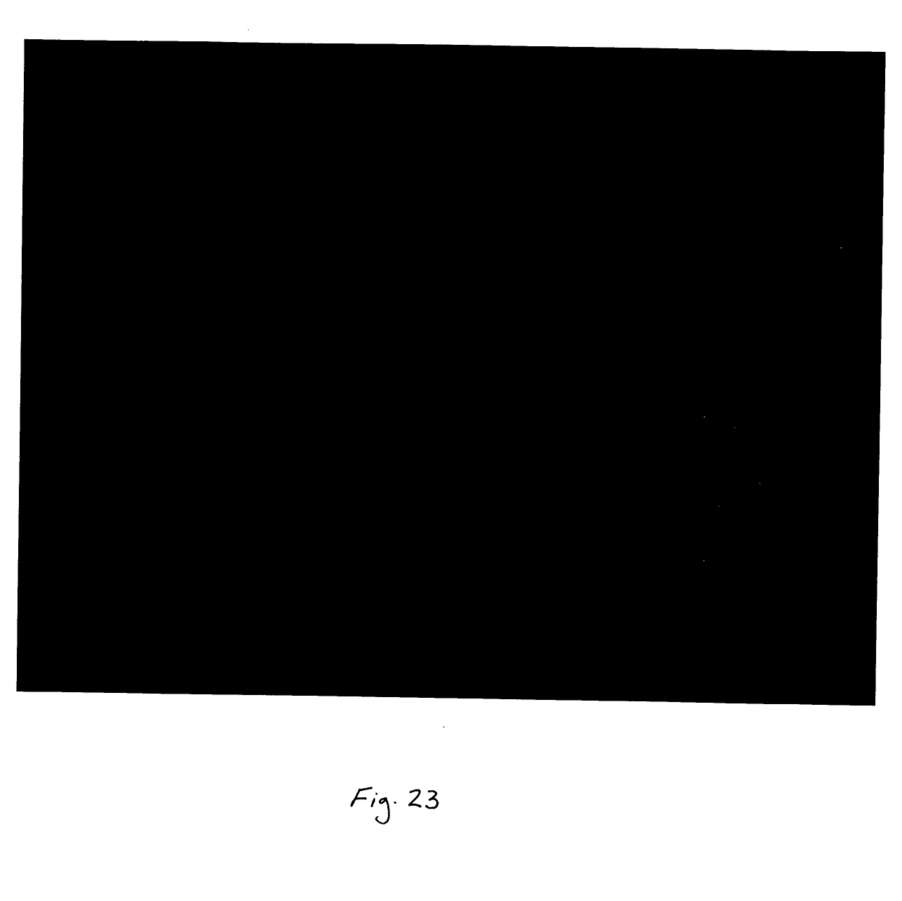

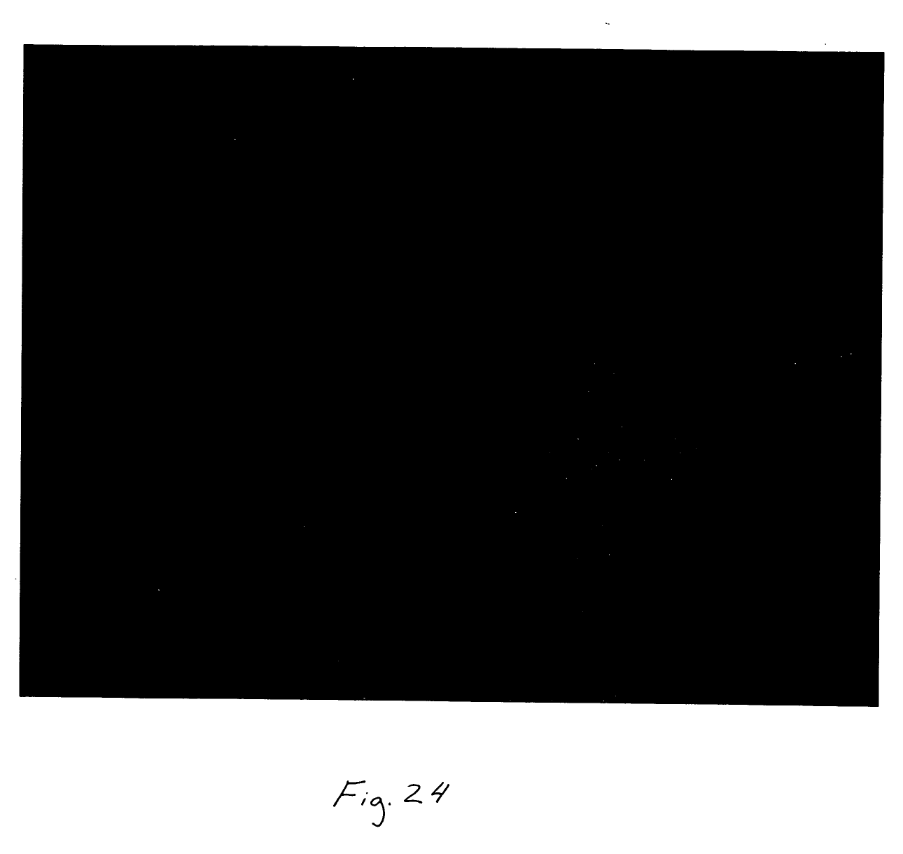

FIG. 22 shows an avian stage one embryo containing Cy3 labeled naked DNA. In FIG. 22 it can be seen that the DNA is localized to certain areas of the embryo. FIG. 23 and FIG. 24 show an avian stage one embryo containing Cy3 labeled DNA coated with low molecular (22 kD) weight PEI (FIG. 23) and high molecular weight (25 kD) PEI (FIG. 24). In FIGS. 23 and 24, it can be seen that the DNA is dispersed throughout the embryos.

These experiments show that DNA/PEI conjugates are distributed more uniformly in the cytoplasm of injected embryos when compared with naked DNA

While this invention has been described with respect to various specific examples and embodiments, it is to be understood that the invention is not limited thereto and that it can be variously practiced with the scope of the following claims.

What is claimed is:

1. A method of producing a transgenic avian comprising:
introducing into an avian cell a nucleic acid comprising a transgene, an integrase activity and a cationic polymer;
introducing the avian cell into a recipient avian wherein the recipient avian produces an offspring which includes the transgene,
thereby producing a transgenic avian.

2. The method of claim 1 wherein introducing the nucleic acid is done by a method selected from the group consisting of microinjecting, transfection, electroporation and lipofection.

3. The method of claim 1 wherein introducing the nucleic acid is done by microinjecting.

4. The method of claim 1 wherein an integrase protein is introduced into the cell.

5. The method of claim 1 wherein a nucleic acid encoding an integrase is introduced into the cell.

6. The method of claim 5 wherein the nucleic acid encoding integrase is mRNA.

7. The method of claim 1 wherein a nuclear localization signal is introduced into the cell.

8. The method of claim 7 wherein the nuclear localization signal is associated with the nucleic acid comprising a transgene.

9. The method of claim 7 wherein the nuclear localization signal is associated with the nucleic acid comprising a transgene by a chemical bond.

10. The method of claim 7 wherein the localization signal is associated with the nucleic acid comprising a transgene by an ionic bond.

11. The method of claim 1 wherein the transgene comprises a coding sequence which is expressed in a cell of the transgenic avian producing a polypeptide.

12. The method of claim 11 wherein the coding sequence is expressed in the blood of the transgenic avian.

13. The method of claim 11 wherein the coding sequence is expressed in the sperm of the transgenic avian.

14. The method of claim 11 wherein the polypeptide is present in egg white produce by the transgenic avian.

15. The method of claim 11 wherein the coding sequence is for a light chain or a heavy chain of an antibody.

16. The method of claim 15 wherein the antibody is a human antibody.

17. The method of claim 11 wherein the coding sequence is for a cytokine.

18. The method of claim 17 wherein the cytokine is interferon.

19. The method of claim 1 wherein the avian cell is an avian embryo cell.

20. The method of claim 1 wherein the avian cell is a cell of an early stage avian embryo comprising a germinal disc.

21. The method of claim 1 wherein the avian cell is an avian embryo cell selected from the group consisting of stage I avian embryo, stage II avian embryo, stage III avian embryo, stage IV avian embryo, stage V avian embryo, stage VI avian embryo, stage VII avian embryo, stage VIII avian embryo, stage IX avian embryo, stage X avian embryo, stage XI avian embryo and stage XII avian embryo.

21. The method of claim 1 wherein the avian cell is a cell of a stage X avian embryo.

22. The method of claim 1 wherein the cationic polymer comprises one or more compounds selected from the group consisting of polyethylenimine, polylysine, DEAE-dextran, starburst dendrimers and starburst polyamidoamine dendrimers.

23. The method of claim 1 wherein the cationic polymer comprises polyethylenimine.

24. The method of claim 1 wherein the avian is a chicken.

25. The transgenic avian produced according to claim 1.

26. An egg produced by a transgenic avian of claim 1.

27. The method of claim 1 wherein the method has an increased efficiency of transgenic avian production relative to an identical method without the integrase or cationic polymer.

28. A method of producing a transgenic avian comprising:
introducing into an avian cell a nucleic acid comprising a transgene, an integrase activity and a nuclear localization signal;
introducing the avian cell into a recipient avian wherein the recipient avian produces an offspring which includes the transgene,
thereby producing a transgenic avian.

29. The method of claim 28 wherein introducing the nucleic acid is done by a method selected from the group consisting of microinjecting, transfection, electroporation and lipofection.

30. The method of claim 28 wherein introducing the nucleic acid is done by microinjecting.

31. The method of claim 28 wherein an integrase protein is introduced into the cell.

32. The method of claim 28 wherein a nucleic acid encoding an integrase is introduced into the cell.

33. The method of claim 32 wherein the nucleic acid encoding integrase is mRNA.

34. The method of claim 28 wherein a nuclear localization signal is introduced into the cell.

35. The method of claim 34 wherein the nuclear localization signal is associated with the nucleic acid comprising a transgene.

36. The method of claim 34 wherein the nuclear localization signal is associated with the nucleic acid comprising a transgene by a chemical bond.

37. The method of claim 34 wherein the localization signal is associated with the nucleic acid by an ionic bond.

38. The method of claim 28 wherein the transgene comprises a coding sequence which is expressed in a cell of the transgenic avian producing a polypeptide.

39. The method of claim 38 wherein the coding sequence is expressed in the blood of the transgenic avian.

40. The method of claim 38 wherein the coding sequence is expressed in the sperm of the transgenic avian.

41. The method of claim 38 wherein the polypeptide is present in egg white produce by the transgenic avian.

42. The method of claim 38 wherein the coding sequence is for a light chain or a heavy chain of an antibody.

43. The method of claim 42 wherein the antibody is a human antibody.

44. The method of claim 38 wherein the coding sequence is for a cytokine.

45. The method of claim 44 wherein the cytokine is interferon.

46. The method of claim 28 wherein the cell is an avian embryo cell.

47. The method of claim 28 wherein the avian cell is a cell of an early stage avian embryo comprising a germinal disc.

48. The method of claim 1 wherein the avian cell is an avian embryo cell selected from the group consisting of stage I avian embryo, stage II avian embryo, stage III avian embryo, stage IV avian embryo, stage V avian embryo, stage VI avian embryo, stage VII avian embryo, stage VIII avian embryo, stage IX avian embryo, stage X avian embryo, stage XI avian embryo and stage XII avian embryo.

49. The method of claim 28 wherein the avian cell is a cell of a stage X avian embryo.

50. The method of claim 28 wherein the cationic polymer comprises one or more compounds selected from the group consisting of polyethylenimine, polylysine, DEAE-dextran, starburst dendrimers and starburst polyamidoamine dendrimers.

51. The method of claim 28 wherein the cationic polymer comprises polyethylenimine.

52. The method of claim 28 wherein the avian is a chicken.

53. The transgenic avian produced according to claim 28.

54. An egg produced by a transgenic avian of claim 28.

55. The method of claim 28 wherein the method has an increased efficiency of transgenic avian production relative to an identical method without the integrase or nuclear localization signal.

56. A method of dispersing nucleic acid in a cell comprising:
introducing into a cell a nucleic acid and a dispersing agent in an amount that will disperse the nucleic acid in a cell.
thereby dispersing nucleic acid in a cell.

57. The method of claim 56 wherein the cell is an avian cell.

58. The method of claim 56 wherein the cell is an embryo cell

59. The method of claim 56 wherein the nucleic acid includes a transgene.

60. The method of claim 56 wherein NLS or integrase activity is introduced into the cell.

61. The method of claim 57 including introducing the avian cell into a recipient avian wherein the recipient avian produces an offspring which includes the transgene,

62. The method of claim 56 wherein the dispersing is a homogeneous dispersing.

63. The method of claim 56 wherein the dispersing agent is a cationic polymer.

64. The method of claim 56 wherein the cationic polymer comprises one or more compounds selected from the group consisting of polyethylenimine, polylysine, DEAE-dextran, starburst dendrimers and starburst polyamidoamine dendrimers.

65. The method of claim 56 wherein the dispersing agent is polyethylenimine.
